(19) United States
(12) Patent Application Publication
(10) Pub. No.: US 2007/0066580 A1
(43) Pub. Date: Mar. 22, 2007

(54) Novel aldosterone antagonists and uses thereof

(75) Inventors: Amedeo Leonardi, Milan (IT); Carlo Riva, Varese (IT); Paolo Tavecchia, Rho (IT); Giorgio Sironi, Pieve Emanuele (IT)

Correspondence Address: DARBY & DARBY P.C., P. O. BOX 5257, NEW YORK, NY, 10150-5257, US

(73) Assignee: Recordati Ireland Limited, Ringaskiddy (IE)

(21) Appl. No.: 11/516,717

(22) Filed: Sep. 5, 2006

Related U.S. Application Data
(60) Provisional application No. 60/714,104, filed on Sep. 2, 2005.

(51) Int. Cl. A61K 31/585; C07J 71/00

(52) U.S. Cl. 514/175; 540/72

(57) ABSTRACT

The present invention relates to novel compounds that are aldosterone receptor antagonists, to pharmaceutical compositions containing the aldosterone receptor antagonists, and to methods of treatment using the same.

### FIELD OF THE INVENTION

The present invention relates to novel compounds that are aldosterone receptor antagonists, to pharmaceutical compositions containing the aldosterone receptor antagonists, and to methods of treatment using the same.

### BACKGROUND OF THE INVENTION

The renin-angiotensin-aldosterone system (RAAS) has been identified as the key regulatory enzymatic cascade for cardiovascular homeostasis and is the current target for many therapeutics designed to ameliorate hypertension and to protect against congestive heart failure (CHF) (Alderman. N. Engl. J. Med. 1991; 324:1098-1104). Activation of RAAS leads to the synthesis of the mineralocorticoid aldosterone, the overproduction of which has been shown to be associated with such conditions as edema, CHF, nephrosis, essential hypertension, cirrhosis of the liver, and severe vascular injury. The most common antihypertensive agents in clinical use are angiotensin-converting enzyme (ACE) inhibitors and angiotensin II type I (AT₁) antagonists, pharmaceuticals that lower blood pressure through a reduction of aldosterone synthesis. It had been widely assumed that the inhibition of the RAAS by an ACE inhibitor will prevent aldosterone formation. However, marked plasma aldosterone levels have been detected, in a majority of patients receiving chronic treatment with an ACE inhibitor. Increasing evidence suggests that ACE inhibitors only transiently suppress aldosterone levels (Struthers. J Cardiac Failure 1996; 2:47-54). Plasma aldosterone levels decrease initially with ACE inhibitor treatment, but return to pretreatment levels after three to six months of therapy, despite good compliance with continued drug administration (Staessen, et al. J Endocr 1981; 91:457-465). This phenomenon, known as “aldosterone escape” occurs because there are other important determinants of aldosterone release, such as serum potassium (Pitt. Cardiovascular Drugs and Therapy 1995; 9:145-149). Marayev, et al. (Presentation at the International Meeting on Heart Failure, 1995, Amsterdam, The Netherlands) have proposed that aldosterone escape could contribute to the high mortality rate in heart failure patients. Aldosterone escape, along with an increased understanding of the role of aldosterone in cardiovascular injury (vide infra) has led to the need for new treatments of cardiovascular disease, including medicines that inhibit processes facilitated by aldosterone itself (i.e., downstream of ACE and AT₁ in RAAS) (Takeda. Hypertension 1995; 120:893-901).

Intracellular receptors (IRs) form a class of structurally-related genetic regulators called “ligand dependent transcription factors” (Evans. Science 1988; 240:889). Steroid receptors are a recognized subset of the IRs. The steroid receptors include the progesterone receptor (PR), androgen receptor (AR), estrogen receptor (ER), glucocorticoid receptor (GR) and mineralocorticoid receptor (MR). The first step in the action of aldosterone is its binding to the mineralocorticoid (i.e., aldosterone) receptor (MR).

When overproduced, aldosterone's action on the MR contributes to hypertension and cardiovascular disease through the retention of sodium and water in the kidney (Brown. Nature Rev. 2003; 2:177-178). Thus, inhibition of MR with an appropriate antagonist re-establishes electrolyte homeostasis to ultimately lower blood pressure through the reduction of vascular volume.

Also, MR activation can contribute to CHF through action in the heart, blood vessels, and brain (Rocha. Ann. NY Acad, Aci. 2002; 970:89-100 and McMahon. Curr. Opin. Pharmacol. 2001; 1:190-196). For example, primary hyperaldosteronism in rat models was shown to promote vascular inflammation, ventricular hypertrophy, and fibrosis (e.g., cardiac, renal, and vascular) due to the stimulation of collagen production. The fibrotic effects of aldosterone are not blocked by ACE treatment alone and therefore occur in the absence of Angiotensin II (hormonal aldosterone precursor in RAAS) due to “escape” (Brilla. J. Mol. Cell. Cardiol. 1993; 25:563-575). Consequently, successful and selective inhibition of MR would not only provide Na⁺/H₂O homeostasis via diuretic effects, but protection against cardiovascular disease through other biological mechanisms associated with aldosterone.

Weber, et al. (Circulation 1987; 75:I-40-47) reported that chronic elevations in circulating aldosterone levels resulted in fibrous tissue formation in the heart and vessels, which contributed to progressive heart failure in selected animals. The increase in myocardial collagen production and subsequent left ventricular hypertrophy lead to myocardial stiffness, reduced ventricular and vascular compliance, impaired diastolic filling, diastolic and systolic dysfunction, ischemia, and ultimately heart failure. According to Struthers (J Cardiac Failure 1996; 2:47-54), such myocardial fibrosis can also lead to arrhythmias and sudden death. Aldosterone blocks myocardial norepinephrine uptake, increases plasma norepinephrine, and promotes ventricular ectopic activity. Rocha, et al. (Am J Hypertension 1999; 12:76A) reported that aldosterone affected baroreceptor function and causes cerebro- and renal-vascular damage as well as endothelial dysfunction in rats. Aldosterone also reportedly increases plasminogen activator inhibitor levels and thereby may impede fibrinolysis.

Greene, et al. (J Clin Invest 1996; 98: 1063-8) have argued that, although much evidence has accumulated to implicate angiotensin II in mediating renal disease, aldosterone also may be associated with progressive renal disease through both hemodynamic effects and direct cellular actions. Hyperaldosteronism and adrenal hypertrophy have been observed in the remnant kidney model, with plasma levels of aldosterone increased approximately tenfold. In addition, clinical studies have reported a relationship between augmented levels of aldosterone and renal deterioration. (Hene, et al. Kidney Int 1982; 21:98-101). Berl, et al. (Kidney Int 1978; 14:228-35), for example, noted that plasma aldosterone levels were elevated in 5 of 8 normokalemic patients with renal failure, and in 5 of 6 patients with a creatinine clearance <15 mL/min. In a subsequent study, Hene, et al. (Kidney Int 1982; 21:98-101) noted that the plasma aldosterone levels in 28 patients with creatinine clearances <50% of normal were increased, despite normal serum potassium levels and normal plasma renin activity. Ibrahim, et al. (Semin Nephrol 1997; 17:43-140) have suggested that it is likely that potassium and angiotensin II (both at increased levels in patients with renal failure) act in concert to promote the aldosterone excess that accompanies renal insufficiency and progressive renal disease. Quan, et al. (Kidney Int 1992; 41:326-33) reported that hypertension, proteinuria, and structural renal injury were less prevalent in rats that underwent subtotal nephrectomy with adrenalectomy compared with rats that had partial nephrectomy but intact adrenal glands. This result occurred despite large doses of replacement glucocorticoid (aldosterone was not replaced) in the adrenalectomized rats.

In a deoxycorticosterone acetate (“DOCA”) salt hypertensive rat model, exogenous administration of mineralocorticoids to DOCA-treated animals induced lesions of malignant nephrosclerosis and stroke (Gavras, et al. Circ Res 1975; 36:300-9). Horiuchi, et al. (Am J Physiol 1993; 264:286-91) reported an increased concentration of aldosterone receptors in the kidneys of a substrain of stroke-prone spontaneously hypertensive rats (“SHRSP”), in which the development of malignant nephrosclerosis occurred without salt-loading. Furthermore, Ullian, et al. (Am J Physiol 1997; 272:1454-61) reported that Wistar-Furth rats (which are unresponsive to the action of aldosterone) are resistant to developing nephropathy in response to subtotal nephrectomy.

Greene, et al. (J Clin Invest 1996; 98:1063-8) evaluated four treatment groups (sham-operated rats, untreated partial-nephrectomized [“remnant”] rats, remnant rats treated with losartan and enalapril, and remnant rats treated with losartan and enalapril followed by an infusion of aldosterone) to distinguish the relative importance of aldosterone in the progression of renal injury. The reported results indicated that remnant rats had a tenfold elevation in aldosterone levels in comparison with sham-operated rats. In contrast, remnant rats undergoing treatment with losartan and enalapril manifested suppressed aldosterone levels, with a decrease in proteinuria, hypertension, and glomerulosclerosis compared with the remnant rats not given these agents. In the final group, remnant rats receiving losartan and enalapril treatment followed by an infusion of aldosterone, the degree of proteinuria, hypertension, and glomerulosclerosis was similar to that of untreated remnant rats.

Control of blood pressure by treatment with antihypertensive drugs has contributed to dramatic reductions in morbidity and mortality attributed to hypertension. For example, age-adjusted death rates from stroke have declined in the United States by nearly 60 percent and from coronary heart disease by 53 percent (Hansson, et al. Lancet 1988; 351:1755-62). According to one report, diastolic blood pressure reduction from 105 to 83 mm Hg could prevent four major cardiovascular events per 1,000 patients treated per year, which would result in 2,764,000 prevented events if the estimated 691 million hypertensive patients received optimal antihypertensive treatment (Hansson, et al. Lancet 1988; 351:1755-62). Even in medically developed countries such as the U.S., however, it is estimated that only 29 percent of patients receiving antihypertensive treatment are controlled below 140/90 mm Hg. (Joint National Committee. “The sixth report of the Joint National Committee on prevention, detection, evaluation, and treatment of high blood pressure.” NIH Publication No. 98-4080. November 1997).

A variety of drugs selected from a number of different drug classes can be used to treat hypertension, heart failure and renal dysfunction (Joint National Committee. “The sixth report of the Joint National Committee on prevention, detection, evaluation, and treatment of high blood pressure.” NIH Publication No. 98-4080. November 1997). These drugs include diuretics (such as chlorthalidone, hydrochlorothiazide, metolazone and the like), vasodilators (such as hydrolazine, minoxidil, sodium nitroprusside, dizaoxide and the like), beta-adrenergic receptor antagonists (such as propranolol, metoprolol, labetalol, acebutolol and the like), calcium channel blockers (such as verapamil, diltiazem, nifedipine and the like), and angiotensin-II receptor antagonists (such as losartan and the like), as well as ACE inhibitors (such as captopril, enalapril, lisinopril, quinapril and the like). The choice of the initial drug therapy for an individual hypertensive patient typically is based on coexisting factors such as age, race, and concurrent diseases (Kaplan. J Hypertension 1995; 13 Suppl 2:S113-S117).

ACE inhibitors are commonly used as standard therapy and have been shown to have a beneficial effect on survival and hospitalization in patients with heart failure. In the CONSENSUS (Cooperative North Scandinavian Enalapril Survival Study) trial, mortality at one year was reduced by 31% in patients with severe heart failure (New York Heart Association Functional Class IV) treated with enalapril (an ACE inhibitor) plus diuretics having no substantial aldosterone (MR) antagonistic activity compared to placebo plus diuretics having no substantial aldosterone (MR) antagonistic activity. In CONSENSUS, patients with high baseline plasma aldosterone levels had a higher mortality than patients with low baseline levels. In the group treated with enalapril, mortality was reduced only in the group with baseline aldosterone plasma levels above the median. In the group whose baseline aldosterone plasma levels were below the median, no difference from placebo in mortality was observed (Swedberg, et al. Circulation 1990; 82:1730-1736). Patients who experience acute myocardial infarction often develop heart failure and subsequently die. Blockade of the RAAS by ACE inhibitors has been shown to reduce all cause mortality in such patients (Lancet 1993; 342:821-828).

The most widely used and widely published aldosterone (MR) antagonist agents are the spirolactones, which are steroidal structures that contain a γ-lactone or a γ-hydroxy acid at the C¹⁷ position. See, for example, Fieser and Fieser. Steroids; page 708 (Reinhold Publ. Corp., New York, 1959) and British Patent Specification No. 1 041 534.

Spironolactone, 7α-acetylthio-20-spirox-4-ene-3,21-dione [See structure (1), below], was the first clinically approved MR antagonist for the treatment of hypertension (see the Merck Index, 10th Edition, 8610; page 1254; Merck & Co., Rahway, N.J., U.S.A.; 1983). Spironolactone acts at the mineralocorticoid receptor level by competitively inhibiting aldosterone binding. This steroidal compound has been used for blocking aldosterone-dependent sodium transport in the distal tubule of the kidney in order to reduce edema and to treat essential hypertension and primary hyperaldosteronism (Mantero, et al. Clin. Sci. Mol. Med. 1973; 45:219s-224). Spironolactone is also used commonly in the treatment of other hyperaldosteronism-related diseases such as liver cirrhosis and congestive heart failure (Saunders, et al. Aldactone; Spironolactone: A Comprehensive Review. Searle, New York. 1978). Progressively-increasing doses of spironolactone from 1 mg to 400 mg per day (i.e., 1 mg/day, 5 mg/day, 20 mg/day) were administered to a spironolactone-intolerant patient to treat cirrhosis-related ascites (Greenberger, et al. N. Eng. Reg. Allergy Proc. 1986; 7:343-345). It has been recognized that development of myocardial fibrosis is sensitive to circulating levels of both Angiotensin II and aldosterone, and that the aldosterone (MR) antagonist spironolactone prevents myocardial fibrosis in animal models, thereby linking aldosterone to excessive collagen deposition (Klug, et al. Am. J. Cardiol. 1993; 71: 46A-54A). Spironolactone has been shown to prevent fibrosis in animal models irrespective of the development of left ventricular hypertrophy and the presence of hypertension (Brilla, et al. J. Mol. Cell. Cardiol. 1993; 25:563-575). Spironolactone at a dosage ranging from 25 mg to 100 mg daily is used to treat diuretic-induced hypokalemia, when orally-administered potassium supplements or other potassium-sparing regimens are considered inappropriate (Physicians' Desk Reference, 46th Edn., p. 2153, Medical Economics Company Inc., Montvale, N.J. 1992).

The effectiveness of known modulators of steroid receptors is often tempered by their undesired side-effect profile, particularly during long-term administration. For example, the effectiveness of progesterone and estrogen agonists, such as norgestrel and diethylstilbesterol respectively, as female birth control agents must be weighed against the increased risk of breast cancer and heart disease to women taking such agents. Similarly, the progesterone antagonist, mifepristone (RU486), if administered for chronic indications, such as uterine fibroids, endometriosis and certain hormone-dependent cancers, could lead to homeostatic imbalances in a patient due to its inherent cross-reactivity as a GR antagonist. Accordingly, identification of compounds which have good specificity for one or more steroid receptors, but which have reduced or no cross-reactivity for other steroid or intracellular receptors, would be of significant value in the treatment of male and female hormone responsive diseases.

Spironolactone was developed as a treatment for hyperaldosteronism which can occur with hypertension and edematous conditions associated with congestive heart failure and liver cirrhosis, but is less commonly used for therapy than ACE inhibitors (Swedberg, et al. Circulation 1990; 82:1730-6). Pitt, et al. (The New England J. of Med. 1999; 341:709-717) recently reported that addition of spironolactone to standard therapy of ACE inhibitor plus loop diuretic having no substantial aldosterone (MR) antagonistic activity reduced morbidity and mortality among patients with severe heart failure. Chronic use of spironolactone, however, is limited in many patients because of its clinical adverse effects, particularly those that are progestational and antiandrogenic in nature resulting in gynecomastia, menstrual abnormalities, and impotence (The RALES Investigators. Am J Cardiol 1996; 78:902-12). Thus a need exists in the art for the development of more selective aldosterone-receptor antagonists, which do not have such undesired side effects.

A class of steroidal-type aldosterone receptor antagonists exemplified by epoxy-containing spirolactone derivatives is described in U.S. Pat. No. 4,559,332 issued to Grob, et al. This patent describes 9α,11α-epoxy-containing spirolactone derivatives as aldosterone receptor antagonists that are useful for the treatment of hypertension, cardiac insufficiency and cirrhosis of the liver. Eplerenone, 7α-acetylthio-9α,11α-epoxy-20-spirox-4-ene-3,21-dione [See structure (2), below], the first FDA approved pharmaceutical in this class, has shown efficacy in blood pressure reduction with no undesirable side-effects resulting from sexual hormone imbalance (i.e., eplerenone is selective for the MR receptor). Eplerenone is especially useful in conjunctive therapy with ACE inhibitors and/or AT₁ antagonists and has been shown to significantly reduce hypertension in patients who were unresponsive to mono-therapy with these more conventional therapeutics.

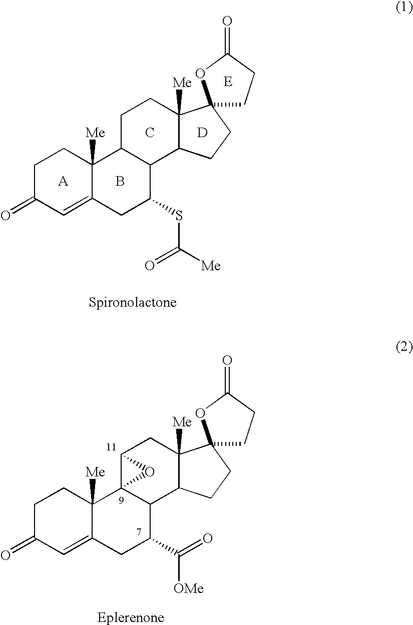

The increased selectivity of eplerenone, as compared to spironolactone, for the mineralocorticoid receptor is derived from two key structural changes to the steroidal framework: i) the addition of an (S,S)-epoxy residue between carbons 9 and 11, and (ii) the substitution at carbon 7 of a methyl ester for a thioacetate. Although eplerenone's enhanced specificity alleviates undesired side effects, it provides a 20 to 30-fold lower affinity for the MR receptor (radioreceptor binding to hMR) and is only 50-75% as potent as spironolactone in vivo (Weinberger. Am. J. Hypertens. 2002; 15:709-716). This reduction in efficacy, vs. spironolactone (1), has provided an impetus for detailed study into structure activity relationships (SARs) to discover more active and selective analogues of the 20-spiroxane steroidal structure.

Great efforts have been taken to develop more specific, potent anti-mineralocorticoids and several compounds were found to be promising. Among these are prorenone/prorenoate (Casals-Stenzel, et al. Arch. Pharmacol Suppl. 1981; 316:R49), mexrenone/mexrenoate (Cutler, et al. Pharmacol. Exp. 1979; 209:144), and spirorenone and 1,2-dihydroxyspirorenone (Laurent, et al. J. Steroid Biochem. 1983; 19:771). The latter two are some of the most potent antimineralocorticoids known (approximately 7.5 times more potent than spironolactone), but in the same range of concentrations, they exert even more pronounced progestogenic effects in man. Mespirenone (CAS 87952-98-5), the delta 1,2-15-beta-16-beta-methylene derivative of spironolactone, also proved to be a potent and quite specific inhibitor of adrenocortical mineralocorticoid synthesis in vitro and shows about three times higher anti-MR effectiveness compared with spironolactone (Losert, W. et al., Drug Res. 36: 1583-1600, 1986), but antagonizes the GR as well. The antimineralocorticoid RU 28318 (the potassium salt of 7α-propyl spirolactone) has also shown to have a higher MR antagonist activity as compared to spirolactone and has a very low affinity for receptors of progesterone and testosterone (Perroteau, et al. J. Steroid Biochem. 1984; 20:853-856).

Improved drug therapies for patients who do not satisfactorily respond to conventional drug therapies used to treat hypertension, heart failure, end-stage renal disease and other pathogenic conditions would be desirable. Further, the increasing prevalence of such pathogenic conditions suggests that newer therapeutic interventions and strategies are needed to replace or complement current approaches. The present invention addresses this need and provides a new drug therapy comprising the administration of one or more steroidal compounds that are aldosterone (MR) antagonists to treat hypertension, heart failure, end-stage renal disease and other pathogenic conditions in a population of subjects characterized by salt sensitivity and/or an elevated dietary sodium intake. The present invention also address the need for selective aldosterone (MR) antagonists that do not interact significantly with the progesterone receptor (PR), androgen receptor (AR), estrogen receptor (ER), or glucocorticoid receptor (GR).

Brown, et al. (J. Med. Chem 1963; 6:732 and J. Org. Chem 1960; 25: 96) disclose a variety of steroids with a spirolactone ring structure for use as aldosterone (MR) antagonists.

Pearlman, et al. (WO 2003/082895) disclose a process for the preparation of eplerenone. Specifically, the publication provides for a synthesis of eplerenone through intermediate compounds with furanyl, propenyl, and carboxylic acid substituents in the 7-α position. Additionally, WO 2004/085458 discloses a method for the synthesis of eplerenone and other spirolactone-derived steroids.

Grob, et al. (Helv. Chim. Acta 1997; 80:566-585) disclose a variety of aldosterone (MR) antagonists including those with a spirolactone ring and with 7-α ester and cyano substitution.

De Gasparo, et al. (J. Pharm. And Exp Therapeutics 1987; 240:650-656) disclose a variety of steroids including those with a spirolactone ring and 7-α thioester substitution.

Patchett, et al. (J. Org. Chem. 1962; 27:3822-3828) disclose a variety of steroids including those with a spirolactone ring and 7-α thioester substitution.

Ng, et al. (U.S. Pat. No. 5,981,744) disclose a processes for preparation of 9,11-epoxy steroids.

Patchett, et al. (U.S. Pat. No. 3,094,552) disclose alkanoylthio and pyrazolo derivates of androstanes substituted with spirolactam and piperazole functionality.

Brown, et al. (U.S. Pat. No. 3,939,155) disclose dihydrospiroandrostanes with antiandrogenic and diuretic activity.

Perroteau, et al. (J. Steroid Biochem. 1984; 20:853-856) disclose the effect of antimineralocorticoid RU 28318 on aldosterone biosynthesis. Additional biological studies with RU 28318 are disclosed by Ulmann, et al. (Eur. J. Clin. Pharmacol. 1985; 28: 531-535).

Szilagyi, et al. (Acta Chimica Hungarica 1984; 116:111-123) disclose a variety of steroid compounds substituted with spirocarbonate and spiroketal ring systems that show antialdosterone activity.

Solyom, et al. (Acta Chimica Acad. Sci. Hung. 1979; 100:89-99) describe novel steroid derivatives substituted with spirooxalozidinethione functionality.

Szilagyi, et al. (U.S. Pat. No. 4,328,221) disclose a process for the preparation of spirooxazolidines derivatives of steroids.

Solyom, et al. (U.S. Pat. No. 4,218,446) disclose a process for the preparation of spirooxazolidinone derivatives of steroids.

Funder, et al. (Biochem. Pharmacol. 1974; 23:1493-1501) report a variety of molecular modifications to the spirolactonized androstane scaffold including the introduction of functionality at the 6, 7, 9, 11 positions of the steroidal framework. Additionally, the reference discloses the affinity of these compounds for the aldosterone receptor.

Weier, et al. (J Med Chem. 1976; 19:975-977) report a variety of spirolactonized androstane scaffold including 7-α cyano and alkoxycarbonylamino derivatives. Additionally, the reference discloses the affinity of these compounds for the aldosterone receptor.

Roux-Schmitt, et al. (Bulletin de la Societe Chimique de France 1986; 1:109-114) disclose the synthesis of 7-α substituted androstane spirolactones including keto-substituted derivatives.

Tweit, et al. (Chemical and Pharmaceutical Bulletin 1964; 12:859-865) disclose the synthesis of 7-α substituted androstane spirolactones including hydroxyl and OAc-substituted derivatives.

Rafestin-Oblin, et al. (J. Steroid Biochem. 1986; 25:527-534) disclose the effect of steroid functional group substitution patterns (SARs) of the mineralocorticoid and glucocorticoid receptors of the rabbit kidney. Specifically spirolactonized steroids with 7-α ester and amide functionalities were studied.

Weier, et al. (J. Med. Chem. 1975; 18:817) disclose the synthesis of 7-α substituted androstane spirolactones including ester derivatives. Additionally, the reference discloses their affinity for the aldosterone receptor.

Nirde, et al. (Molecular Pharmacology 2001; 59:1307-1313) disclose the synthesis of 11-β substituted androstane spirolactones and ring-opened lactone derivatives thereof including ester derivatives. Additionally, the reference describes the compounds as a class of aldosterone agonists

Kandemirli, et al. (Il Farmaco 2002; 57:601-607) describe a variety of cyclopropyl substituted spirolactone-derived steroids and an electron-topological approach to their structure-activity relationship (SAR) with regard to the mineralocorticoid receptor. An expanded SAR study is presented by Kandemirli, et al. (Arzneim-Forsch./Drug Res. 2003; 53:133-138).

Grassy, et al. (J. of Molecular Graphics 1995; 13:356-367) describe a the molecular and quantum modeling for the SAR of 54 spirolactone-derived steroid homologs with regard to the mineralocorticoid receptor.

Mercier, et al. (J. Pharm. Belg. 1995; 50:223-230) disclose the use of a topological correlation search for aldosterone (MR) antagonists and agonists and its application to a class of 52 spirolactone and hydroxymethylcarbonyl steroids.

Cella, et al. (U.S. Pat. No. 2,918,463) disclose a class of androstane steroids derivatized with spirolactone functionality of 5-6 ring atoms. The compounds are further described in J. Org. Chem. 1959; 24:743-748.

Sharma, et al. (J. Pharmaceutical Sciences 1971; 69:1677-1682 disclose a variety of potential aldosterone (MR) antagonists with spirooxazole functionality. The synthesis of these compounds is also disclosed therein.

Magnus, et al. (J. Am. Chem. Soc. 1978; 100:7746-7747) describe the synthesis of a variety of spirofuranone substituted steroids using an allene cyclization reaction.

Weindel, et al. (Arzneim.-forsch./Drug Res. 1991; 41:1082-1091) provide a SAR analysis of a group of 27 different C-17 spirosteroids performed to uncover the role of these compounds in the late steps of aldosterone biosynthesis.

Green, et al. (U.S. Pat. No. 3,968,132) disclose the synthesis of a series of cyclobutanone substituted androstanes.

Patchett, et al. (U.S. Pat. No. 3,103,510) disclose methods for the preparation of spirolactone derived androstanes wherein the spirolactone is further substituted with a halide. Brown, et al. (U.S. Pat. No. 3,438,979) disclose further modification of the spirolactone derived androstanes wherein the spirolactone is further substituted with alkylamino groups. Spirolactone derived androstanes wherein the spirolactone is further substituted with alkyl and alkylcarboxylic acid groups are described by Creger (J. Org. Chem. 1972; 37: 1907-1918).

Fretland, et al. (J. Steroid. Biochem. 1985;22:305-310) disclose a SAR analysis between spirolactone derived steroids and their effect on phospholipase/aldosterone synthetase.

### SUMMARY OF THE INVENTION

In one embodiment, the present invention relates to aldosterone receptor antagonists of formula I: 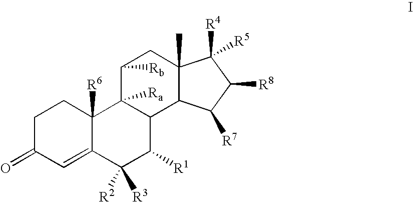
wherein

R¹ is selected from hydrogen, alkyl, alkenyl, alkynyl, cycloalkyl, carboxyl, cyano, tetrazolyl, isoxadiazolyl, alkylisoxadiazolyl, cycloalkylthio, —C(═O)O-alkyl, —C(═O)-alkyl, —C(═O)NHR^{x}, —C(═O)N(alkyl)(R^{x}), —C═N—OR^{x}, —C═N—O(aralkyl), —C(═O)N(R^{x})(O-alkyl), —CH₂OR^{x}, —CH₂NHR^{x}, —CH₂N(alkyl)(R^{x}), OR^{x}, OC(═O)NH-alkyl, —OC(═O)N(alkyl)₂, OC(═O)alkyl, —SR^{x}, —SC(═O)-alkyl, —S(═O)-alkyl, —SO₂-alkyl, —S-alkyl-C(═O)O-alkyl, —S-alkyl-N(R^{y})C(═O)-alkyl, —S-alkyl-CF₃, —S-alkyl-N(R^{y})₂, —NHR^{x}, —N(alkyl)(R^{x}), —N(R^{y})C(═O)-alkyl, —N(R^{y})C(═O)O-alkyl, N(R^{y})—C(═O)NHR^{x}, —N(R^{y})—SO₂-alkyl, and —N—(R^{y})C(═O)—CF₃;

R² is selected from hydrogen and halogen;

R³ is hydrogen or hydroxymethyl (CH₂OH), or

R² and R³ when taken together with the carbon to which they are attached form a cyclopropyl ring system

R⁴ and R⁵ when taken together form a ring system selected from

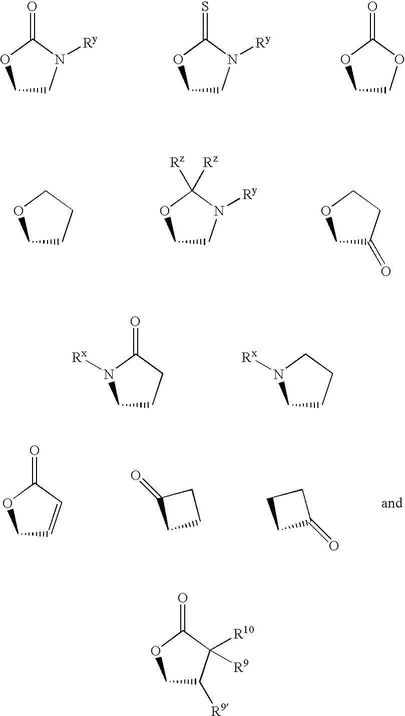

Ra and Rb are not present and there is a C═C between carbons 9 and 11 or;

Ra and Rb together form a bridge A between carbons 9 and 11, wherein
A is selected from —O—, —S—, —CH₂—, and —CF₂—;

R⁶ is selected from hydrogen, alkyl, —CH₂OR^{x}, —CH₂SR^{x}, —CH₂SO-alkyl, —CH₂SO₂-alkyl, —CH₂NHR^{x}, —CH₂N(alkyl)(R^{x}), —C(═O)O-alkyl, —C(═O)-alkyl, —C(═O)NHR^{x}, and C(═O)N(alkyl)(R^{x});

R⁷ and R⁸ are hydrogen or when taken together with the carbon to which they are attached form a cyclopropyl ring system

R⁹ and R^{9′} are independently selected from hydrogen, halo, alkyl, and —C(═O)O-alkyl;

or R⁹ and R^{9′} are not present and there is a C═C between carbons 22 and 23

R¹⁰ is selected from hydrogen and —C(═O)OR^{x};

R^{x} is selected from hydrogen, alkyl, and acyl;

R^{y} is selected from hydrogen and alkyl;

R^{z} is selected from hydrogen and alkyl;

wherein

when R¹ contains an alkyl or alkenyl substituent, said alkyl or alkenyl substituent in R¹ can optionally and independently be substituted by one or more of —OR^{x}, —NHR^{x}, —N(alkyl)(R^{x}), halogen, —C(═O)N(R^{y})₂, —N(R^{y})₂, aryl, —C(═O)—Oalkyl, OC(═O)NHR^{x}, —OC(═O)alkyl, —OC(═O)Oalkyl, OC(═O)alkyl-O-alkyl, and OC(═O)alkyl-O—Ac; and

pharmaceutically acceptable salts, solvates, prodrugs and metabolites thereof;

with the following provisos:

(i) R¹ is not —C(═O)O-alkyl, —C(═O)OH or hydrogen when R² is hydrogen, Ra and Rb form an oxo bridge or are not present and there is a C═C between carbons 9 and 11 and R⁴ and R⁵ together form

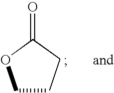

(ii) R¹ is not —S—C(═O)—CH₃ when R² is hydrogen, Ra and Rb form an oxo bridge and R⁴ and R⁵ together form

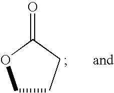

(iii) R¹ is not cyano when R² is hydrogen, Ra and Rb are not present and there is a C═C between carbons 9 and 11, and R⁴ and R⁵ together form

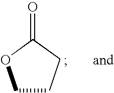

(iv) R¹ is not hydrogen when R² is hydrogen, Ra and Rb form an oxo bridge or are not present and there is a C═C between carbons 9 and 11, and R4 and R5 together form

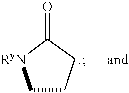

(v) Ra and Rb do not together form an oxo bridge or there is not a C═C between carbons 9 and 11 when R¹ is hydrogen or alkanoylthio, R² is hydrogen, and R⁴ and R⁵ together form

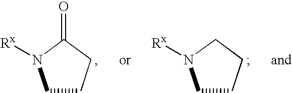

(vi) R¹ is not 2-propenyl or 3-butenyl when R² is hydrogen, Ra and Rb are not present and there is a C═C between carbons 9 and 11, and R4 and R5 together form

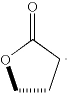

In a second embodiment, the present invention relates to pharmaceutical compositions containing one or more compounds of formula I.

In a third embodiment, the present invention relates to the use of one or more compounds of formula I for the treatment of a condition that may be regulated or normalized via inhibition of the mineralocorticoid receptor.

In a fourth embodiment, the present invention relates to the use of one or more compounds of formula I for the treatment of a condition that may be regulated or normalized via inhibition of the mineralocorticoid receptor while not modulating the effects of the glucocorticoid, progesterone, androgen, or estrogen receptors.

A further embodiment of the present invention is the use of one or more compounds of formula I for the treatment of diseases or disorders associated with the overproduction of aldosterone.

A further embodiment of the present invention is the use of one or more compounds of formula I for the treatment of aldosteronism.

A further aspect of the present invention is the use of one or more compounds of formula I for the treatment of hypertension.

A further aspect of the present invention is the use of one or more compounds of formula I for the treatment of heart failure and left ventricular hypertrophy.

A further aspect of the present invention provides a method of inhibiting aldosterone receptor activity comprising contacting an aldosterone receptor with an effective amount of one or more compounds of Formula I. The aldosterone receptor is preferrably a mammalian aldosterone receptor and most preferrably a human aldosterone receptor.

A further aspect of the present invention provides a method of treating aldosteronism comprising administering to a patient in need of such treatment an effective amount of one or more compounds of Formula I.

A further aspect of the present invention provides a method of treating aldosteronism comprising administering to a patient in need of such treatment an effective amount of one or more compounds of Formula I, wherein the aldosteronism is selected from the group consisting of primary hyperaldosteronism and secondary hyperaldosteronism.

A further aspect of the present invention provides a method of treating aldosteronism comprising administering to a patient in need of such treatment an effective amount of one or more compounds of Formula I, wherein the aldosteronism is selected from the group consisting of hypertension, cardiovascular disease, renal dysfunction, edema, cerebrovascular disease, insulinopathies, stroke, Type II diabetes mellitus, heart failure and left ventricular hypertrophy.

A further aspect of the present invention provides for a pharmaceutical composition comprising one or more compounds of Formula I and, optionally, a pharmaceutically acceptable carrier.

A further aspect of the present invention provides for a pharmaceutical composition of comprising one or more compounds of Formula I, further comprising a second active agent. The second active agent is preferably selected from the group consisting of renin inhibitors, angiotensin II antagonists, ACE inhibitors, calcium channel blockers, diuretics, and retinoic acid.

### BRIEF DESCRIPTION OF THE FIGURES

FIG. 1 sets forth the complete amino acid sequence for the GAL4 DNA binding domain—human mineralocorticoid ligand binding domain fusion protein (GAL4 DBD-hMR LBD) (SEQ ID NO: 19). Amino acid residues of the GAL4 DBD (SEQ ON NO: 16) are indicated by underlining. Amino acid residues of the hMR LBD (SEQ ID NO: 14) are indicated by bold typeface.

### DETAILED DESCRIPTION OF THE INVENTION

As described in more detail below, the invention provides novel compounds that are aldosterone receptor antagonists. The invention further provides methods of treating aldosteronism, including primary aldosteronism and secondary aldosteronism, using the novel compounds of the invention, as well as pharmaceutical compositions comprising the novel compounds of the invention.

Definitions:

As used herein:

The terms “hydroxy” and “hydroxyl” are synonymous and refer to a group —OH;

The terms “amino” and “amine” are synonymous and refer to a group —NH₂;

The terms “keto” and “oxo” are synonymous and refer to the group ═O;

The term “oxo bridge” refers to an oxygen atom that is bound to two atoms or groups, —O—;

The terms “epoxy” and “epoxide” are synonymous and refer to an oxo bridge joining two adjacent carbon atoms such to form a three membered heterocyclic ring system;

The term “carbonyl” refers to a group —C(═O)—;

The term “carboxyl” refers to a group —CO₂H and consists of a carbonyl and a hydroxyl group (More specifically, C(═O)OH);

The terms “imino” and “imine” are synonymous and refer to a group —C(═N)—;

The term “cyano” refers to a group —C≡N;

The terms “halo” or “halogen” are synonymous and refer to fluoride, chloride, bromide, or iodide atoms;

The term “thio” refers to a group —S—;

The terms “sulfonyl” and “sulfon” are synonymous and refer to a group —S(═O)₂—;

The term “sulfoxyl” refers to a group —SO—;

The term “tetrazolyl” refers to any heterocyclic ring structure containing four nitrogen atoms and one carbon atom;

The term “trifluoromethyl” refers to a group —CF₃; and

The terms “sulfonamido” and “sulfonamide” are synonymous and refer to the group —SO₂NH—;

The term “alkyl” as used herein as a group or a part of a group refers to a straight or branched hydrocarbon chain containing the specified number of carbon atoms. For example, C₁₋₁₀ alkyl means a straight or branched alkyl containing at least 1, and at most 10, carbon atoms. Examples of “alkyl” as used herein include, but are not limited to, methyl, ethyl, n-propyl, n-butyl, n-pentyl, isobutyl, isopropyl, t-butyl, hexyl, heptyl, octyl, nonyl and decyl. A C₁₋₄ alkyl group is preferred, for example methyl, ethyl, n-propyl, isopropyl, n-butyl, isobutyl or t-butyl.

The term “cycloalkyl” group as used herein refers to a non-aromatic monocyclic hydrocarbon ring of 3 to 8 carbon atoms such as, for example, cyclopropyl, cyclobutyl, cyclopentyl, cyclohexyl or cycloheptyl.

The term “substituted alkyl” as used herein denotes alkyl radicals wherein at least one hydrogen is replaced by one more substituents such as, but not limited to, hydroxy, alkoxy, aryl, heterocycle, halogen, trifluoromethyl, pentafluoroethyl, cyano, cyanomethyl, nitro, amino, amide, amidine, amido, carboxamide, carbamate, carbonate, ester, alkoxyester, acyloxyester and the like. The definition pertains whether the term is applied to a substituent itself or to a substituent of a substituent.

The term “substituted cycloalkyl” as used herein denotes cycloalkyl radicals further bearing one or more substituents as set forth herein.

The term “alkoxy” as used herein refers to a straight or branched chain alkoxy group containing the specified number of carbon atoms. For example, C₁₋₆ alkoxy means a straight or branched alkoxy containing at least 1, and at most 6, carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy and hexyloxy. A C₁₋₄ alkoxy group is preferred, for example methoxy, ethoxy, propoxy, prop-2-oxy, butoxy, but-2-oxy or 2-methylprop-2-oxy.

The term “alkenyl” as used herein as a group or a part of a group refers to a straight or branched hydrocarbon chain containing the specified number of carbon atoms and containing at least one double bond. For example, the term “C₂₋₆ alkenyl” means a straight or branched alkenyl containing at least 2, and at most 6, carbon atoms and containing at least one double bond. Examples of “alkenyl” as used herein include, but are not limited to, ethenyl, 2-propenyl, 3-butenyl, 2-butenyl, 2-pentenyl, 3-pentenyl, 3-methyl-2-butenyl, 3-methylbut-2-enyl, 3-hexenyl and 1,1-dimethylbut-2-enyl. It will be appreciated that in groups of the form —O—C₂₋₆alkenyl, the double bond is preferably not adjacent to the oxygen.

The term “alkynyl” as used herein as a group or a part of a group refers to a straight or branched hydrocarbon chain containing the specified number of carbon atoms and containing at least one triple bond. For example, the term “C₂₋₆ alkynyl” means a straight or branched alkynyl containing at least 2, and at most 6, carbon atoms and containing at least one triple bond. Examples of “alkynyl” as used herein include, but are not limited to, ethynyl, 2-propynyl, 3-butynyl, 2-butynyl, 2-pentynyl, 3-pentynyl, 3-methyl-2-butynyl, 3-methylbut-2-ynyl, 3-hexynyl and 1,1-dimethylbut-2-ynyl. It will be appreciated that in groups of the form —O—C₂₋₆alkynyl, the triple bond is preferably not adjacent to the oxygen. The term “substituted alkenyl” refers to an alkenyl wherein at least one hydrogen is replaced by one or more substituents or groups independently selected for each position.

As used herein, the term “aryl”, alone or in combination, means an unsaturated aromatic carbocyclic group having 6-14 carbon atoms having a single ring such as phenyl or multiple fused rings such as naphthyl. Aryl may optionally be further fused to an aliphatic or aryl group or can be substituted with one or more substituents such as halogen (fluorine, chlorine and/or bromine), hydroxy, C₁-C₇ alkyl, C₁-C₇ alkoxy or aryloxy, C₁-C₇ alkylthio or arylthio, alkylsulfonyl, cyano or primary or nonprimary amino.

As used herein, the term “alkanoyl” and “acyl” are synonymous and refer to a straight or branched alkyl chain attached to a carbonyl group. The alkanoyl radicals may be substituted or unsubstituted. Examples of “alkanoyl” groups include, but are not limited to, formyl, acetyl (ethanoyl), propanoyl, isopropanoyl, butanoyl, isobutanoyl, valeryl (pentanoyl), isovaleryl, pivaloyl, hexanoyl, t-butanoyl, pentanoyl, 3-methylpentanoyl or the like. Preferred alkanoyl radicals are “lower alkanoyl” radicals having about one to five carbon atoms, such as formyl, acetyl or propanoyl.

As used herein, the term “acetyl” refers to an alkanoyl group of two carbon atoms (i.e., a methyl group attached to a carbonyl group). “Acetyl” is represented by the formula, C(═O)-methyl.

The term “dialkylamino” denotes an amino group in which the N atom of the amino (as defined above) is twice substituted with alkyl (as defined above) radicals. Preferably, the alkyl radicals are independently one to six carbon atoms in length.

The term “furanyl”, alone or in combination, refers to a five-membered heteromonocyclic group with four carbons and one oxygen and where the ring system is fully unsaturated. Furanyl groups are defined as heteroaryl and may be substituted or unsubstituted.

The term “alkylthio”, alone or in combination, refers to a straight or branched alkyl chain having from one to six carbon atoms attached to a sulfur atom. Examples of alkylthio groups include, but are not limited to, methylthio, ethylthio, propylthio, isopropylthio, butylthio and the like.

The term “spirocyclic” and “spirocycle” are synonymous and refer to two ring systems that are fused together at a common atom. Spirocycles are typically formed upon the joining of two ring systems at one tetrahedral carbon atom as the spirocenter. Each ring system can be the same or different, can be fused to other ring systems, and consist of cycloalkyl and heterocycloalkyl.

The term “heterospirocyclic” and “heterospirocycle” are synonymous and refer to two ring systems that are fused together at a common atom as described above. Each ring system can be the same or different, can be fused to other ring systems, and one or both contain heteroatoms (e.g., N, O, S) within the ring structure.

The term “spirocenter” refers to the atom of common junction between the two rings of a spirocycle. The atom is preferably carbon but can be any atom that is tetravalent and assumes a tetrahedral geometry at the ring junction (e.g., N as an ammonium salt).

Exemplary compounds of formula I are set forth in Table 1, below (R6 is methyl for each compound represented in Table 1)

TABLE 1

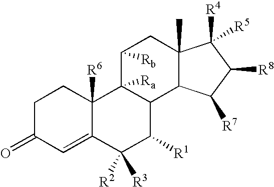

| R1 | R2/R3 | Ra/Rb | R7/R8 | R4/R5 |
|---|---|---|---|---|
| —SEt | H; H | —O— | H; H | 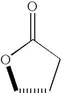 |
| —CH₂CH₂CH═CH₂ | H; H | double bond | H; H | 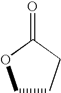 |
| Propyl | H; H | double bond | H; H | 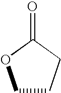 |
| Propyl | H; H | —O— | H; H | 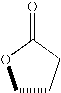 |
| —SC(═O)Me | H; H | double bond | H; H | 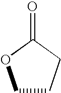 |
| —SPr | H; H | —O— | H; H | 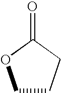 |
| —SEt | H; H | double bond | H; H | 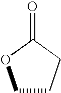 |
| —SO₂Et | H; H | double bond | H; H | 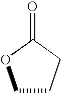 |
| —C(═O)NMe₂ | H; H | —O— | H; H | 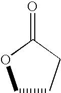 |
| —C(═O)NHMe | H; H | —O— | H; H | 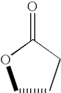 |
| —SCH₂CF₃ | H; H | double bond | H; H | 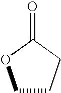 |
| H | H; H | double bond | H; H | 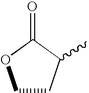 |
| H | H; H | double bond | H; H | 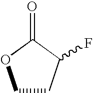 |
| H | H; H | double bond | H; H | 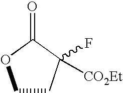 |
| —NH2 | H; H | —O— | H; H | 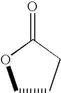 |
| —NHPr | H; H | —O— | H; H | 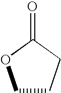 |
| —C═N—OBn | H; H | double bond | H; H | 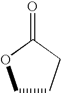 |
| —S(═O)Et | H; H | double bond | H; H | 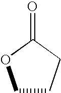 |
| —CH₂NHEt | H; H | double bond | H; H | 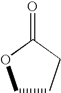 |
| Me | H; H | double bond | H; H | 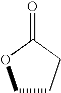 |
| —SCH₂CF₃ | H; H | —O— | H; H | 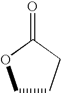 |
| —SC(═O)CH₃ | H; H | double bond | H; H | 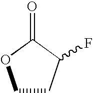 |
| —SCH₂CH(OH)CH₃ | H; H | double bond | H; H | 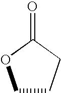 |
| —CH₂N(Et)(C(═O)CH₃) | H; H | double bond | H; H | 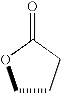 |
| —SCH₂CH₂NMe₂ | H; H | double bond | H; H | 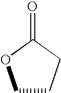 |
| —C(═O)Pr | H; H | —O— | H; H | 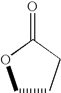 |
| —NHC(═O)CF₃ | H; H | —O— | H; H | 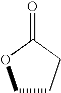 |
| —CH═NOMe | H; H | double bond | H; H | 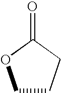 |
| —CH₂OC(═O)NHMe | H; H | double bond | H; H | 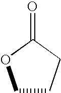 |
| —CH₂OH | H; H | double bond | H; H | 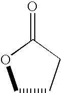 |
| H | H; H | double bond | H; H | 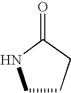 |
| H | H; H | double bond | H; H | 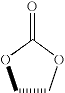 |
| H | H; H | double bond | H; H | 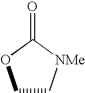 |
| H | H; H | double bond | H; H | 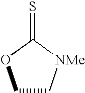 |
| H | H; H | —O— | H; H | 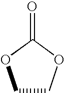 |
| —SC(═O)Me | H; H | double bond | H; H | 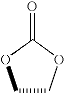 |
| —SC(═O)Me | H; H | double bond | H; H | 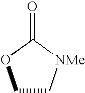 |
| H | H; H | double bond | H; H | 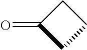 |
| H | H; H | double bond | H; H | 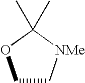 |
| —SC(═O)Me | H; H | —O— | H; H | 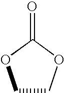 |
| H | H; H | double bond | H, H | 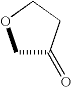 |
| —C(═O)OMe | F; H | —O— | H; H | 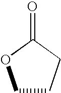 |
| —C(═O)NH₂ | H; H | —O— | H; H | 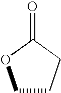 |
| —CN | H; H | —O— | H; H | 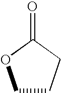 |
| tetrazolyl | H; H | double bond | H; H | 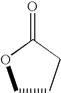 |
| —CH═CH—C(═O)OMe | H; H | —O— | H; H | 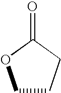 |
| —CH═N—OMe | H; H | —O— | H; H | 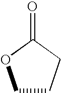 |
| —S(cyclopentyl) | H; H | double bond | H; H | 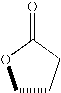 |
| SCH₂CH₂NHC(═O)Me | H; H | double bond | H; H | 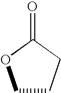 |
| SCH₂C(═O)OMe | H; H | double bond | H; H | 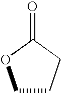 |
| —C(═O)N(Me)(OMe) | H; H | —O— | H; H | 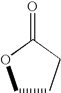 |
| —S(O)₂Et | H; H | —O— | H; H | 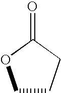 |
| —H | H; H | double bond | H; H | 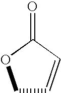 |
| —CH₂OMe | H; H | double bond | H; H | 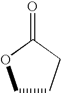 |
| —CH₂OC(═O)OMe | H; H | double bond | H; H | 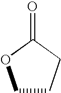 |
| —CH₂OC(═O)CH₂OMe | H; H | double bond | H; H | 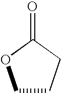 |
| —CH₂OC(═O)CH₂OC(═O)Me | H; H | double bond | H; H | 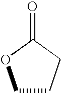 |
| —NHC(═O)Me | H; H | —O— | H; H | 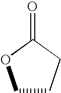 |
| —NHC(═O)OMe | H; H | —O— | H; H | 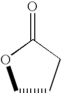 |
| —NHC(═O)NHMe | H; H | —O— | H; H | 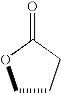 |
| —NHS(O)₂Me | H; H | —O— | H; H | 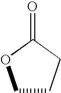 |
| —CH2—CH2—CH3 | H; H | double bond | —CH2— | 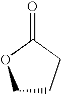 |
| —CH2—CH2—CH3 | H; H | —O— | —CH2— | 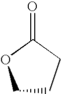 |
| —H | H; H | double bond | —CH2— | 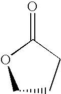 |
| —COOMe | H; —CH2OH | —O— | —H— | 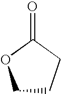 |
| —CN | H; H | double bond | —CH2— | 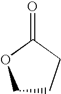 |
| —COOMe | H; H | double bond | —CH2— | 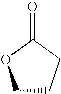 |
| —COOMe | —CH2—CH2— | —O— | H; H | 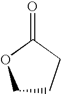 |
| —COOMe | H; H | —O— | —CH2— | 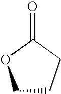 |
| —CH═N—OCH3 (7β steroisomer) | H; H | double bond | —CH2— | 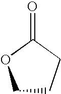 |
| —CH═N—OCH3 (7α steroisomer) | H; H | double bond | —CH2— | 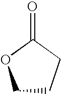 |
| —COOMe | H; —CH2OH | double bond | —CH2— | 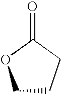 |
| —CH═N—OCH3 | H; H | double bond | —CH2— | 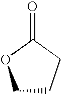 |
| —CH═N—OCH3 | H; H | —O— | —CH2— | 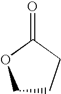 |

### Preparation of the Novel Compounds of the Invention

Compounds of formula I and derivatives thereof may be prepared by the general methods outlined hereinafter, said methods constituting a further aspect of the invention. In the following description, the groups R¹ to R¹⁰, R^{a}, R^{b}, R^{x}, R^{y}, R^{z}, and A have the meaning defined for the compounds of formula I unless otherwise stated.

It will be appreciated by those skilled in the art that it may be desirable to use protected derivatives of intermediates used in the preparation of the compounds of formula I. Protection and deprotection of functional groups may be performed by methods known in the art. Hydroxyl or amino groups may be protected with any hydroxyl or amino protecting group (for example, as described in Green and Wuts. Protective Groups in Organic Synthesis. John Wiley and Sons, New York, 1999). The protecting groups may be removed by conventional techniques. For example, acyl groups (such as alkanoyl, alkoxycarbonyl and aryloyl groups) may be removed by solvolysis (e.g., by hydrolysis under acidic or basic conditions). Arylmethoxycarbonyl groups (e.g., benzyloxycarbonyl) may be cleaved by hydrogenolysis in the presence of a catalyst such as palladium-on-carbon.

The synthesis of the target compound is completed by removing any protecting groups, which are present in the penultimate intermediate using standard techniques, which are well-known to those skilled in the art. The deprotected final product is then purified, as necessary, using standard techniques such as silica gel chromatography, HPLC on silica gel and the like, or by recrystallization.

A further object of the present invention relates to the preparation of compounds of formula I according to processes comprising:

### Preparation of the Spiro Ring System

(a) For compounds of formula I wherein R4 and R5 when taken together form a ring system selected from 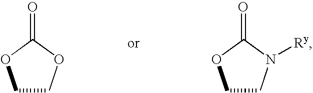
cyclization of a compound of Formula S1 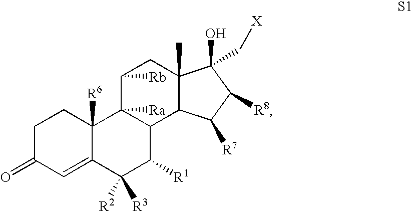
wherein
X is selected from hydroxy or —NR^{y},
with diphosgene, triphosgene, or a compound of Formula S2, 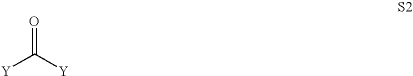
wherein Y is selected from halogen, alkoxy, or imidazole.

For the compounds of Formula S1,

wherein X is OH,

epoxide opening of a compound of Formula S3 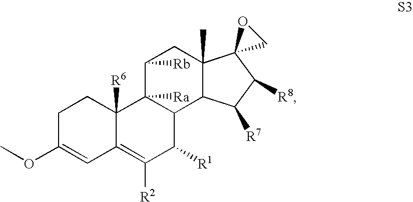
wherein
R¹ and R² are hydrogen,
with sodium hydroxide or an equivalent aqueous base followed by an acid treatment to cleave the enol ether and restore the original α,β-unsaturated ring A ketone.

For the compounds of Formula S1,

wherein X is —NR^{y},

epoxide opening of a compound of Formula S3 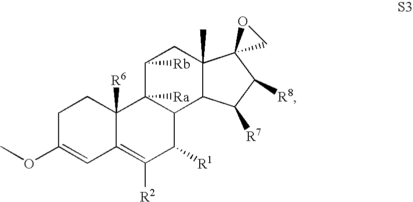
wherein
R¹ and R² are hydrogen,
with alkyl amine in the presence of an organic acid followed by the treatment with water to restore the original α,β-unsaturated ring A ketone.

The compounds of Formula S3 can be prepared as indicated in WO 97/21720 and WO9825948.

(b) For compounds of formula I wherein R4 and R5 when taken together form a ring system selected from 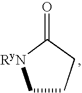
and
R^{y} is hydrogen,
reaction of a compound of Formula S4 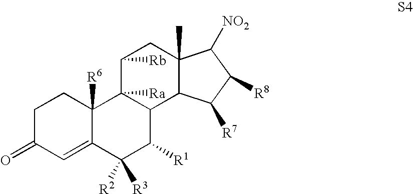
with a compound of Formula S5 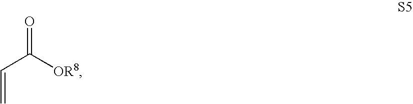
wherein
R⁸ is alkyl,
followed by reduction and concomitant cyclization with Zn.

For the compounds of Formula S4

wherein R¹ and R² are hydrogen,

17-hydroxylamine formation of a compound of Formula S6 followed by oxidation with a organic peracid in organic solvent-water mixture.

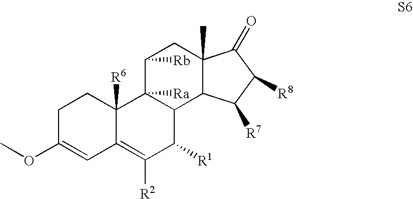

The compounds of Formula S6 can be prepared as indicated in WO 97/21720.

(c) For compounds of formula I wherein R4 and R5 when taken together form the ring system 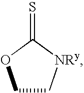
cyclization of a compound of Formula S1 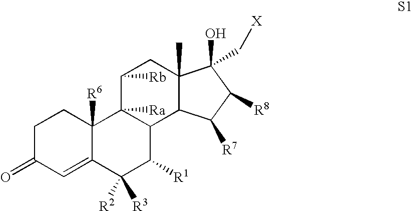
wherein
X is —NR^{y},
with a compound of Formula S7 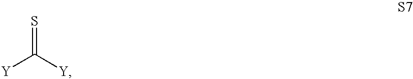
wherein Y is selected from halogen, alkoxy, or imidazole.

(d) For compounds of formula I wherein R4 and R5 when taken together form a ring system selected from 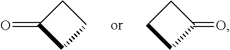
treatment of a compound of Formula S8 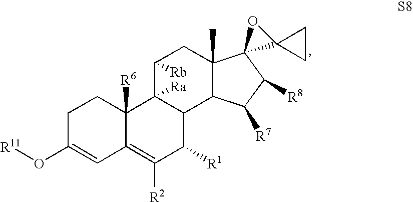
wherein
R¹ and R² are hydrogen and
R¹¹ is alkyl,
with tetrafluoroboric acid.

The compounds of Formula S8 can be prepared as described in U.S. Pat. No. 3,968,132

(e) For compounds of formula I wherein R4 and R5 when taken together form the ring system 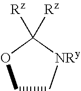
and R^{z} is selected from hydrogen or alkyl;
reaction of a compound of Formula S1,
wherein X is nitrogen;
with paraformaldehyde or a compound of Formula S2, wherein Y is selected from alkyl.

(f) For compounds of formula I wherein R4 and R5 when taken together form the ring system 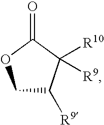
R⁹ and R^{9′} are hydrogen and R¹⁰ is C(═O)—OR^{x},
reaction of a compound of Formula S9 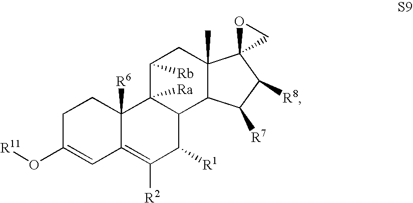
wherein
R¹ and R² are hydrogen; and
R¹¹ is alkyl,
with a compound of Formula S10, 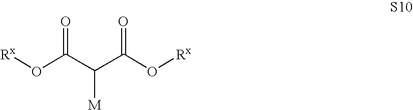
wherein R^{x} is alkyl and
M is hydrogen or an alkali metal,
in the presence of base.

The compounds of Formula S9 can be prepared as described in WO 97/21720

(g) For compounds of formula I wherein R4 and R5 when taken together form the ring system 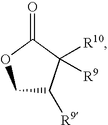
R⁹ is halo or alkyl, R^{9′} is hydrogen, and R¹⁰ is hydrogen or C(═O)OR^{x}, Treatment of a compound of Formula S11, 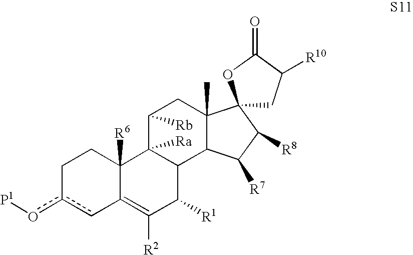
wherein,
P¹ is a protecting group for a ketone or alcohol; and
R¹ and R² are hydrogen,
with base to form the spirolactone enolate, followed by reaction with a halogen, or an alkyl or halo electrophile (e.g., MeI, perchloryl fluoride, 1-Chloromethyl-4-Fluoro-1,4-Diazoniabicyclo[2.2.2]Octane Bis-(Tetrafluoroborate) and analogues, NCS, or NBS), followed by removal of the protecting group with concomitant isomerization to form the requisite α,β-unsaturated ring A ketone. The compounds of Formula S9 are compounds of Formula I that can be prepared according to method (f) above.

(h) For compounds of formula I wherein R4 and R5 when taken together form the ring
system 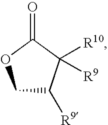
and R^{9′} is alkyl or C(═O)OR^{x}, oxidation of a compound of Formula S12 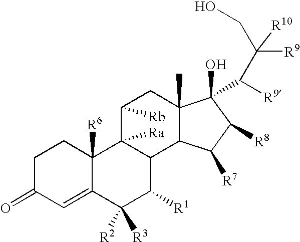
with a suitable oxidizing agent to convert the primary alcohol to a carboxylic acid, and subsequent cyclization (e.g., in situ lactonization) to form the spirolactone. The compounds of Formula S12 can be made from the commercially available compound S6 as illustrated in the following scheme: 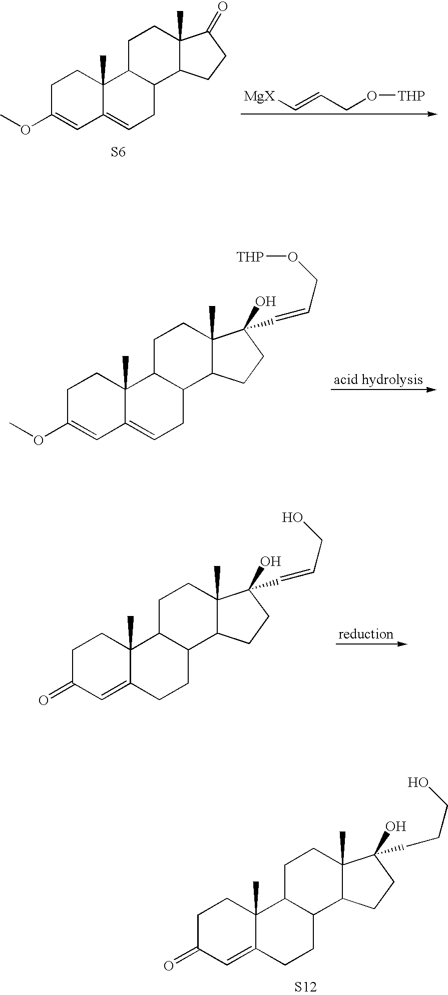
Tetrahydropyranyl protected 3-hydroxypropenyl-1-magnesium halide is added to the compound S6 to form the corresponding allylic alcohol. The allylic alcohol intermediate is then deprotected and the methyl enol ether converted to the corresponding ketone with acid (e.g., HCl, MeOH, THF). The compound of Formula S12 is then made through reduction of the allylic alcohol with, for example, H₂ in the presence of 5% Pd/C.

(i) For compounds of formula I wherein R4 and R5 when taken together form the ring system 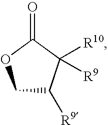
and, R⁹, R^{9′} and R¹⁰ are hydrogen,
hydrogenation of a compound of Formula S13 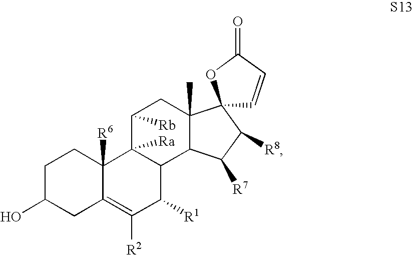
wherein
R¹ and R² are hydrogen
with hydrogen in the presence of a palladium catalyst, and subsequent oxidation and isomerization to form the requisite α,β-unsaturated ring A ketone. The compounds of Formula S13 can be made from the commercially available dehydroepiandrosterone as illustrated in the following scheme: 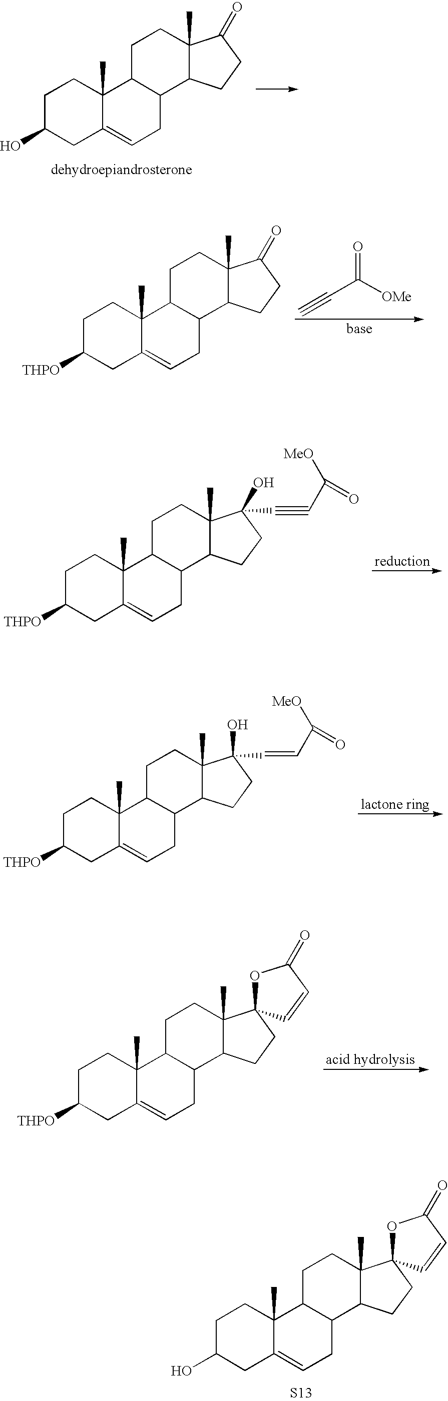
Dehydroepiandrosterone is first protected as its tetrahydropyranyl ether upon treatment with dihydropyran in the presence of catalytic TsOH. The steroid intermediate is then reacted with methyl propynoate in the presence of an appropriate base for alkyne deprotonation (e.g., LDA) to form the alkynyl substituted steroid intermediate. The alkyne is then reduced to the corresponding alkene upon treatment with Red-Al (e.g., 2 equiv. in THF). . . . Finally, the lactone ring is formed and the THP ether deprotected upon treatment with mild acid (e.g., HCl in MeOH).

(j) For compounds of formula I wherein R4 and R5 when taken together form the ring system 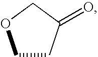
oxidation of a compound of Formula S14 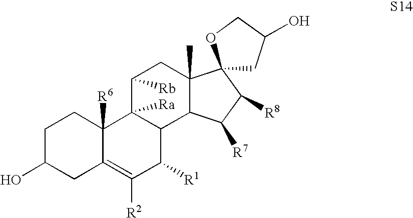
with a suitable oxidant (e.g., Jones' Reagent) and subsequent isomerization to form the requisite α,β-unsaturated ring A ketone.

(k) For compounds of formula I wherein R4 and R5 when taken together form the ring system 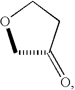
treatment of a compound of Formula S15 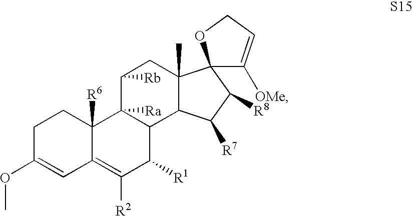
wherein
R¹ and R² are hydrogen,
with a mild acid hydrolysis to convert the protected enol ethers to the corresponding ketones.

The compounds of Formula S15
can be prepared by reacting a compound of Formula S16 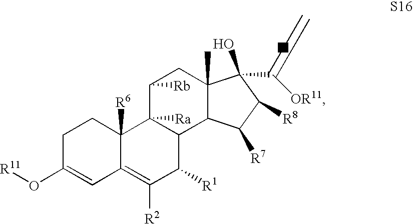
wherein
R¹ and R² are hydrogen and
R¹¹ is alkyl,
with a base such as sodium or potassium alkoxide and in the presence of a crown ether.

The compounds of Formula S16,

wherein

R¹ and R² are hydrogen and

R¹¹ is alky,

can be prepared by reacting a compound of Formula S17 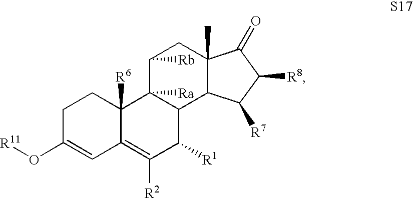
wherein
R¹ and R² are hydrogen and
R¹¹ is alkyl,
with a compound of Formula S18 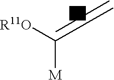
wherein
R¹¹ is alkyl and
M is alkali metal.

The compounds of Formula S17 can be prepared as described in WO 97/21720

(l) For compounds of formula I wherein R4 and R5 when taken together form the ring system 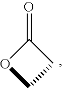
reaction of a compound of Formula S6 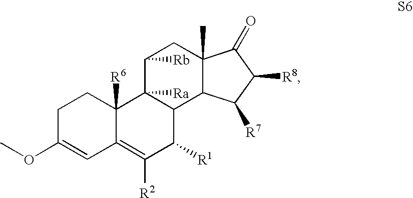
wherein
R¹ and R² are hydrogen,
with a silyl ketene acetal of Formula S18, 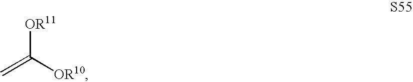
wherein
R¹⁰ is selected from alkyl, and
R¹¹ is selected from trialkylsilyl,
followed by treatment with mild acid for lactonization and to convert the protected enol ether to the corresponding ketone.

(m) For compounds of formula I wherein R4 and R5 when taken together form the ring system 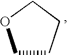
olefin metathesis of a compound of Formula S19 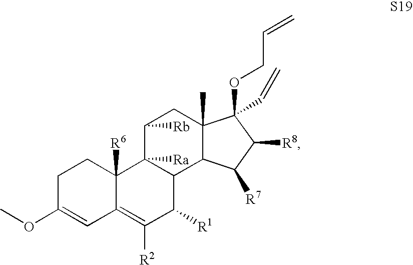
wherein
R¹ and R² are hydrogen,
with a ruthenium catalyst suitable for olefin metathesis,
followed by treatment with mild acid to convert the protected enol ether to the corresponding ketone,
and then hydrogenation of the resultant dihydrofuran with molecular hydrogen and an appropriate palladium catalyst (e.g., 5% Pd/C). The compounds of Formula S19 can be made in two steps from the compounds of Formula S6: (1) vinyl grignard addition to form a tertiary allyl alcohol, and (2) reaction of the alcohol with an allyl halide or triflate to form the compounds of Formula S19.

(n) For compounds of formula I wherein R4 and R5 when taken together form the ring system 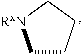
olefin metathesis of a compound of Formula S20 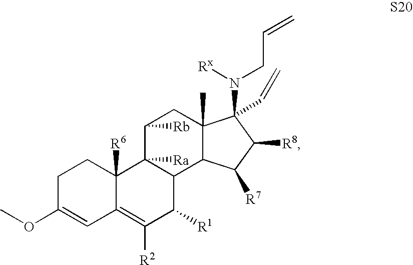
wherein
R¹ and R² are hydrogen,
with a ruthenium catalyst suitable for olefin metathesis,
followed by treatment with mild acid to convert the protected enol ether to the corresponding ketone, and then hydrogenation of the resultant dihydrofuran with molecular hydrogen and an appropriate palladium catalyst (e.g., 5% Pd/C). The compounds of Formula S20 can be made in two steps from the compounds of Formula S6: (1) imine formation with the appropriate allyl amine, and (2) addition of a vinyl grignard to the imine in the presence of a lewis acid.

### Preparation of the 7-Substituted Compounds

The introduction of the substituents at position 7α (R1) may be accomplished pursuing the following approaches that begin from common 6,7-ene intermediates of Formula S22. The groups Ra, Rb, R⁴, R⁵, R⁶, R⁷ and R⁸ have the meaning defined for the compounds of formula I unless otherwise stated.

(a) For preparation of intermediate compounds of Formula S22 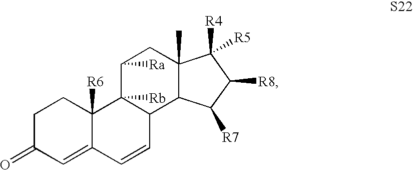
introduction of a double bond at position 6,7 of a compound of Formula S21 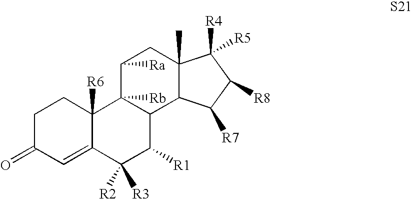
where R¹-R³ are hydrogen by selective oxidation of the single bond at position 6,7 using DDQ or chloranil.

(b) The intermediate compounds of Formula S22 can be also prepared from a compound of Formula S23

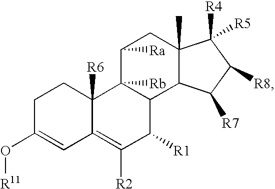

wherein R¹ and R² are hydrogen and

R¹¹ is alkyl,

using palladium acetate by the method of Saegusa (J. Org. Chem. 1978, 43, 10119)

(c) For compounds of formula I wherein R¹ is alkyl, alkenyl, alkynyl, cycloalkyl,

R⁶ is selected from methyl or hydroxymethyl, and A is oxygen,

Michael addition of the compound of Formula S24
R¹-MX, S24
wherein
R¹ is alkyl, alkenyl, alkynyl, cycloalkyl,
M is metal and,
X is not present or is halogen,
to a compound of Formula S22 in the presence of cupreous salt in order to avoid side reactions such as addition to the ketone in position 3 or to the carbonyl in the R4, R5 ring system.

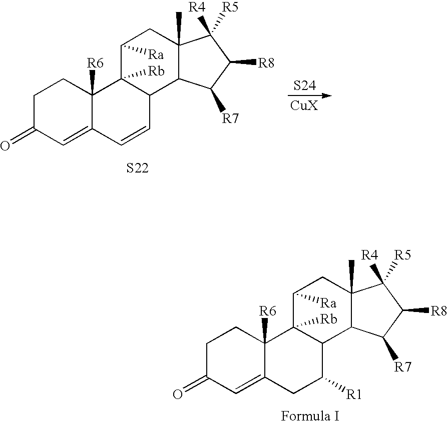

(d) For compounds of formula I wherein R1 is selected from SR^{x}, SC(═O)-alkyl, S-alkyl-C(═O)-alkyl, S-alkyl-C(═O)O-alkyl, S-alkyl-C(═O)—NHR^{x}, S-alkyl-N(R^{y})C(═O)-alkyl, S-alkyl-CF₃, S-alkyl-N(R^{y})₂, R6 is selected from methyl or hydroxymethyl, and A is oxygen,

Michael addition of a compound of Formula S25
R¹—H, S25
wherein
R1 is SR^{x}, SC(═O)-alkyl, S-alkyl-C(═O)-alkyl, S-alkyl-C(═O)O-alkyl,
S-alkyl-C(═O)—NHR^{x}, S-alkyl-N(R^{y})C(═O)-alkyl, S-alkyl-CF₃, S-alkyl-N(R^{y})₂,
to a compound of Formula S22 in the presence of a reducing metal (e.g., sodium) or Lewis Acid (e.g., InCl₃)

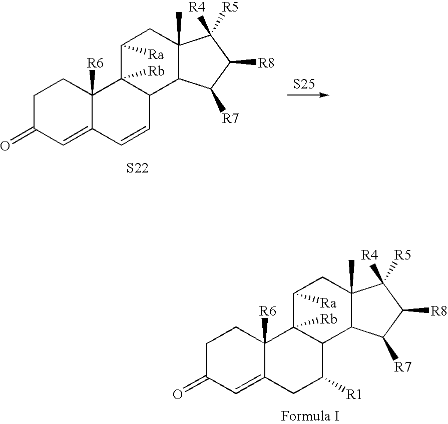

(e) For compounds of formula I wherein R1 is SO—R^{x}, SO₂R^{x}, R3 is selected from methyl or hydroxymethyl, R^{x} is selected from hydrogen, alkyl,
oxidation of the compound of Formula S26 with the methods known in the art such as treatment with H₂O₂ to convert —SR^{x} to —SOR^{x} and subsequent treatment with KMnO4 to convert SOR^{x} to —SO₂R^{x} (March, J. Advanced Organic Chemistry, Third Edition, pg. 1089 and referecens therein). 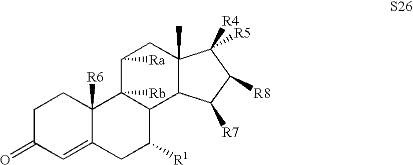
wherein
R1 is S—R^{x},
R6 is selected from methyl or hydroxymethyl,
R^{x} is selected from hydrogen, alkyl, and
A is oxygen, and prepared from S22 using method (d) above,
to give a compound of Formula S27 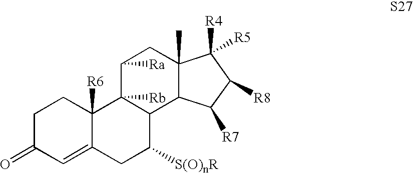
wherein
R is alkyl and,
n is an integer from 1 to 2.

(f) For compounds of formula I wherein R1 is CH₂OR^{x}, R⁶ is selected from methyl, CH₂OR^{x}, A is selected from oxygen, R^{x} is selected from hydrogen,
reduction of a compound of Formula S28: 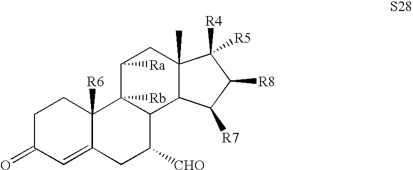
with a commercially available reducing agents such as lithium aluminium hydride or sodium boro hydride or equivalent followed by a selective oxidation of the 3 hydroxyl of the corresponding diols with MnO₂ or other reagents in the art known to oxidize an allylic alcohol without affecting the primary hydroxyl group to give a compound of Formula S29.

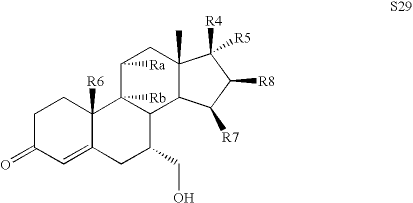

The keto-aldehyde S28 can be prepared as described by Grob in Helvetica Chimica Acta (1997, vol. 80, 566-585).

(g) For compounds of formula I wherein R1 is CH₂OR^{x}, R⁶ is selected from methyl, CH₂OR^{x}, A is selected from oxygen and R^{x} is selected from alkyl, acyl, reaction of a compound of Formula S29 with an appropriate organic or inorganic base to form the corresponding alkoxide, followed by reaction with an alkyl electrophile (e.g., MeI) or an acyl halide or alkyl anhydride to give a compound of Formula S30.

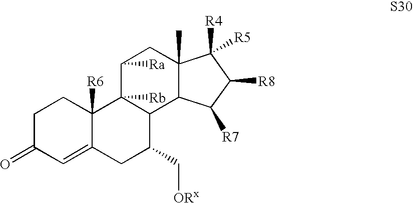

(h) Compounds of formula I wherein R1 is CH₂SR^{x}, CH₂NHR^{x}, CH₂N(alkyl)(R^{x}),

R⁶ is selected from methyl, CH₂OR^{x}, A is selected from oxygen, and R^{x} is selected from alkyl and acyl,

nucleophilic displacement of the sulfonate of a compound of Formula S31 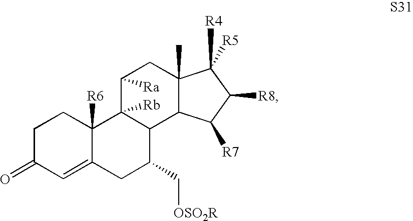
wherein R is methyl or trifluormethyl or tolyl,
with a compound of Formula S32
R—H S32
wherein R is SR^{x}, NHR^{x}, N(alkyl)(R^{x}).

Compounds of Formula S31 can be prepared from a compound of Formula S29 by treating the free alcohol with the appropriate chlorosulfonate or sulfonate anhydride (e.g., CH₃SO₂Cl or CF₃SO₂OSO₂CF₃) in the presence of base (e.g., NEt₃).

(i) Compounds of formula I wherein R1 is C═N—OR^{x}, R⁶ is selected from methyl, CH2OR^{x}, A is selected from oxygen, and R^{x} is selected from alkyl, alkyl-aryl, alkyl-heteroaryl, and acyl,

reaction of a compound of Formula S28

with a compound of Formula S33
H₂N—OR^{x} S33
wherein
R^{x} is selected from alkyl; alkyl-aryl, alkyl-heteroaryl, acyl,
by the methods known in the art suitable to prepare oximes. (March, J. Advanced Organic Chemistry, Third Edition, pg. 805 and referecens therein).

(l) Compounds of formula I wherein R1 is C(═O)-alkyl, R⁶ is selected from methyl, CH₂OR^{x}, A is selected from oxygen, and R^{x} is selected from alkyl; alkyl-aryl, alkyl-heteroaryl, and acyl
by reaction of a compound of Formula S34 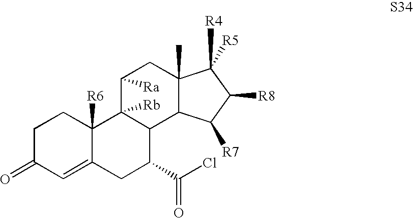
with a compound of Formula S35
R-M-X S35
wherein,
R is alkyl,
M is selected from magnesium or zinc and X is halogen, or
X is not present and M is lithium;
optionally in the presence of a Pd(0) catalyst

The compounds of Formula S34
can be prepared from a compound of Formula S36 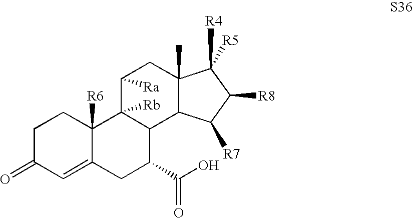
by conversion of the carboxylic acid to the corresponding acid chloride upon treatment with thionyl chloride or oxalyl chloride.
The 7-α carboxylic compound of Formula S36 can be prepared as described in WO 98/25948

(m) Compounds of formula I wherein R1 is C(═O)NHR^{x}, C(═O)N(alkyl)(R^{x}), C(═O)NH—NHR^{x}, C(═O)NH—N(alkyl)(R^{x}), C(═O)N(R^{x})(O-alkyl), R⁶ is selected from methyl, CH₂OR^{x}, A is selected from oxygen, and R^{x} is selected from alkyl; alkyl-aryl, alkyl-heteroaryl, and acyl,

condensation of a compound of Formula S34 with a compound of Formula S37
HN—R¹R² S37
wherein,
R¹ and R² are selected independently from hydrogen, alkyl, N-alkyl, —NHR^{x}, O-alkyl, and R^{x}.

(n) For Compounds of formula I wherein R1 is NHR^{x}, N(alkyl)(R^{x}), N(R^{y})C(═O)-alkyl, N(R^{y})C(═O)O-alkyl, N(R^{y})C(═O)NHR^{x}, N(R^{y})SO₂-alkyl, N(R^{y})C(═O)—CF₃, R⁶ is selected from methyl, CH₂OR^{x} A is selected from oxygen, and R^{x} is selected from alkyl; alkyl-aryl, alkyl-heteroaryl, acyl,
Reaction of a compound of Formula S38 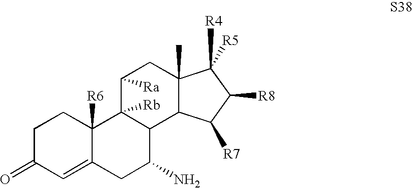
independently with an alkyl halide, acyl halide, R^{x}—NCO, R^{x}—OCO-halide, alkyl-sulfonyl halide, or a trifluoroacetic anhydride.

The compounds of Formula S38 can be prepared via a “Curtius reaction” from a compound of Formula S36; transformation of the carboxylic group into the corresponding acyl-azide and subsequent transposition to give the corresponding isocyanate can be accomplished by a three step procedure involving 1) conversion of the carboxylic acid to the corresponding acid chloride with treatment of thionyl chloride, 2) treatment with NaN₃ to form the carboxylic azide, 3) decomposition of the carboxylic azide with the application of heat (e.g., decompostion in refluxing benzene) to form the nitrene with concomitant rearrangement to the isocyanate and 4) acid hydrolysis of the isocyanate to afford the amine compound of formula S38.

(o) Compounds of formula I wherein R1 is OR^{x}, OC(═O)—NHakyl, OC(═O)—N(akyl)₂, or O(C═O)-alkyl, R⁶ is selected from methyl, CH₂OR^{x}, A is selected from oxygen, and R^{x} is selected from alkyl; alkyl-aryl, alkyl-heteroaryl acyl,
Reaction of compound of Formula S39 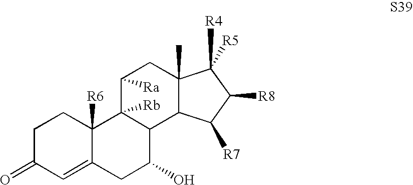
with a strong base (e.g.: NaH) in a polar-aprotic solvent followed by an alkyl halide or an anhydride or an acyl chloride for preparing the corresponding 7-alpha O-alkyl or 7-alpha O-acyl compounds.

Reaction of compound of Formula S39 with phosgene or carbonyl diimidazole followed by an alkylamine or an di-alkylamine provides the corresponding 7-alpha monoalkyl carbamate and 7-alpha di-alkylcarbamate respectively.

The 7-alpha monoalkyl carbamate can be also prepared by reacting compound of Formula S39 with alkylisocyanate.

The compound of Formula S39 can be prepared by reaction of compound of Formula S28 with perbenzoic acid to perform the Baeyer-Villiger reaction followed by ester hydrolysis with an appropriate base (e.g., NaOH).

Alternatively, compound of Formula S39 can be prepared from a compound of Formula S40 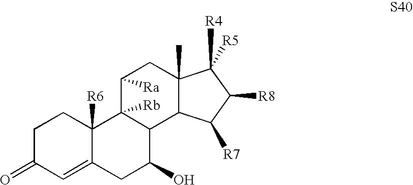
by inversion of the stereochemistry at position 7 according to the Mitsonobu procedure in the presence of acetic acid followed by a mild basic hydrolysis.

The inversion of the 7-beta OH can be also accomplished by nucleophilic displacement of the sulfonate of Formula S41 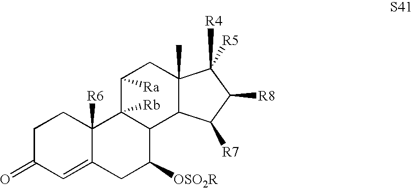
wherein R is methyl or trifluoromethyl or tolyl
with a compound of Formula S42
R′—COO⁻M⁺ S42
wherein R′ is alkyl, aryl and
M⁺ is an alkali metal ion (e.g.: Li⁺, K⁺,).

The mild basic hydrolysis of the corresponding 7-alpha O-acyl provides compound of Formula S39.

Compound of Formula S41 can be prepared from a compound of Formula S40 by treating the free alcohol with the appropriate chlorosulfonate or sulfonate anhydride (e.g. CH₃SO₂Cl or CF₃SO₂OSO₂CF₃) in the presence of base (e.g.: NEt₃).

The compound of Formula S40 can be prepared as described in U.S. Pat. No. 4,079,054.

(p) Compounds of Formula I wherein R6 is CH₂OR^{x}, R1, R2 and R3 are hydrogen, Ra and Rb are not present and there is a C═C double bond between carbons 9 and 11 or Ra and Rb together form a bridge A between carbons 9 and 11, wherein A is oxygen,
Reaction of compound of Formula S43 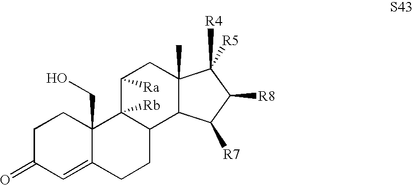
with a strong base (e.g.: NaH) in a polar-aprotic solvent followed by an alkyl halide or an anhydride or an acyl chloride for preparing the corresponding O-alkyl or O-acyl compounds. Compound of Formula S43 can be prepared from a commercially available 11-beta-hydroxy dehydro epiandrosterone S44 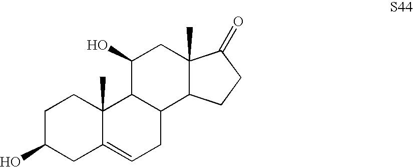
applying, after a suitable protections, the well established procedure providing for 5,6 bromidrine formation (N-bromosuccinimide, HClO₄) followed by the introduction of the 6,19 epoxy moiety using a procedure of Costa (Tetrahedron Letters 1999, 40(49), 8711-8714); the final treatment with zinc and acetic acid (Indian. J. Chem. 1971, 9, 740) give rise to the 19 hydroxy 3-keto-4-ene system.

(q) Compounds of Formula I wherein R6 is CH₂SR^{x}, R1, R2 and R3 are hydrogen, Ra and Rb are not present and there is a C═C double bond between carbons 9 and 11 or Ra and Rb together form a bridge A between carbons 9 and 11, wherein A is oxygen,
Reaction of compound of Formula S45 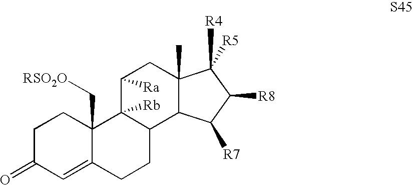
wherein R is methyl or trifluoromethyl or tolyl
with compound of Formula S46
R^{x}—X S46
wherein X is SH

Compound of Formula S45 can be prepared from a compound of Formula S43 by treating the free alcohol with the appropriate chlorosulfonate or sulfonate anhydride (e.g. CH₃SO₂Cl or CF₃SO₂OSO₂CF₃) in the presence of base (e.g.: NEt₃).

(r) Compounds of Formula I wherein R6 is —CH₂SO-alkyl, —CH₂SO₂ alkyl, R1, R2 and R3 are hydrogen, Ra and Rb are not present and there is a C═C double bond between carbons 9 and 11 or Ra and Rb together form a bridge A between carbons 9 and 11, wherein A is oxygen, reaction of the thioether resulting from reaction of a compound of Formula S45 with a compound of Formula S46 with a suitable oxidant that transforms a thioether into the corresponding sulphoxide (e.g., KMnO₄) and/or sulphone (e.g., H₂O₂).

(s) Compounds of Formula I wherein R6 is —CH₂NHR^{x}, —CH₂N(alkyl)(R^{x}), R1, R2 and R3 are hydrogen, Ra and Rb are not present and there is a C═C double bond between carbons 9 and 11 or Ra and Rb together form a bridge A between carbons 9 and 11, wherein A is oxygen, reaction of compound of Formula S45 with compound of Formula S47
R—X S47
wherein X is NH₂ or NH(alkyl)

(t) Compounds of Formula I wherein R6 is —C(═O)O-alkyl, —C(═O)NHR^{x}, —C(═O)N(alkyl)R^{x}, R1, R2 and R3 are hydrogen, Ra and Rb are not present and there is a C═C double bond between carbons 9 and 11 or Ra and Rb together form a bridge A between carbons 9 and 11, wherein A is oxygen,
reaction of compound of Formula S48 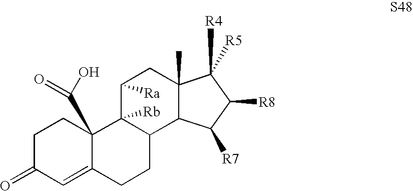
with reagents able to transform the carboxylic group into the corresponding chloride (e.g.: SOCl₂) and then with compound of Formula S49
R—X S49
wherein
R is alkyl and X is selected from OH, NH₂ and NH(alkyl)

Compound of Formula S48 can be prepared from compound S43 with reagents suitable to transform a primary alcohol into the corresponding carboxylic acid (e.g.: KMnO₄).

(u) Compounds of Formula I wherein R6 is —C(═O)-alkyl, R1, R2 and R3 are hydrogen, Ra and Rb are not present and there is a C═C double bond between carbons 9 and 11 or Ra and Rb together form a bridge A between carbons 9 and 11, wherein A is oxygen,
reaction of compound of Formula S50 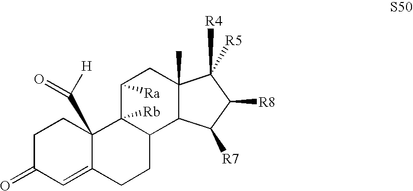
with a compound of Formula S51
R-MX S51
wherein
R is alkyl
M is metal and,
X is not present or is halogen

The resulting secondary alcohol is then oxidized to the corresponding ketone with a suitable reagent such as Martin's compound or PDC, or PDD.

Compound of Formula S50 can be prepared from compound of Formula S43 by oxidation of the primary alcohol into the corresponding aldheyde using an appropriate oxidant (e.g., Swern reagent or Dess-Martin periodinate).

(v) Compounds of Formula I wherein Ra and Rb together form a bridge A between carbons 9 and 11, wherein A is selected from —S—, —CH₂—, —CF₂—
reaction of compound of Formula S52 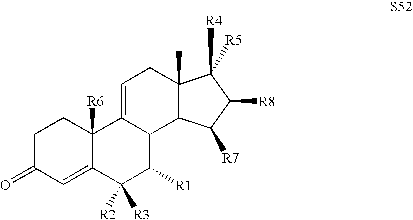
with diethoxythioxophosphoranesulphenyl bromide and tetrabutylammonium fluoride (SYNLETT 1994, 4, 267-268) for preparing the 9,11 episulfide
with Et₂Zn/CH₂I₂ for preparing the 9,11 cyclopropane
with CBr₂F₂/triphenylphosphine, 18-crown-6 and KF for preparing the 9,11 difluorocyclopropane.

### Preparation of 15,16-Beta Cyclopropyl Compounds

Compounds of Formula I bearing the cyclopropyl group at positions 15,16 with beta stereochemistry (R⁷═R⁸═CH₂) may be prepared pursuing the following approaches that begin from common compounds of Formula S53. The groups Ra, Rb, R1, R2, R6 have the meaning defined for the compounds of formula I unless otherwise stated. 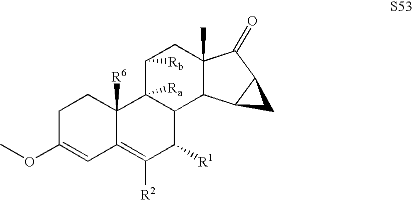
To compound S54, introduce first a cyclopropyl at position 15,16 using trimethylsulfoxonium iodide and sodium hydride in a mixture of THF-DMSO at room temperature and then introduction of the beta epoxide at position 17 by treatment with trimethylsulfonium iodide

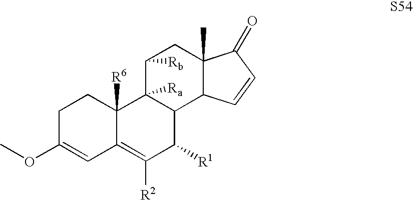

(b) Intermediate compounds of Formula S54 can be prepared from compounds of Formula S6 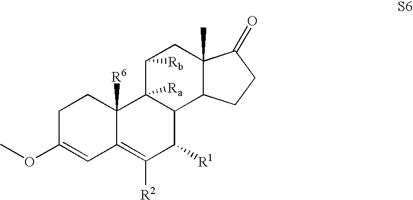
by 1) addition of a phenylselenyl group at position 16 with the methods known in the art such as treatment of compounds of Formula S6 with a strong organic base such as lithium dimethylamide or equivalent and phenylselenyl bromide 2) oxidation of the phenylselenyl group to the corresponding phenylselenyl oxide, and 3) treatment of the crude phenyl selenyl oxide with an organic base at reflux or by other methods known in the art suitable for prepare a ketone α-β unsaturated system starting from a ketone.

### Preparation of 6-hydroxymethyl and 6,6 ′spiro-cyclopropyl Compounds

Compounds of Formula I (R²═H and R³═CH₂OH) bearing an hydroxy methyl group at position 6 may be prepared as indicated in Von Marcel Müller et al, Helv. Chim. Acta., 1980, vol 63, part 7, 1867-1889 or Klaus Annen et al, Justus Liebigs Annalen der Chemie, 1983, vol 4, pages 705-711 by reacting compounds of Formula I (R² and R³═H) with pyrrolidine in alcoholic solvent (e.g.: methanol) at reflux for about half an hour. The corresponding 3-pyrrolidinyl-3,5 diene intermediate is then treated with formaldehyde (37% in water) in a 2:1 ethanol/toluene mixture at room temperature yielding to the desired 6-hydroxymethyl compounds.

Compounds of Formula I (R² and R³═CH₂—CH₂) bearing an ethylene bridge at positions 6,6′ may be prepared as indicated in J. Med. Chem. 1991, 34, 2464-2468 from compounds of Formula I (R²═H and R³═CH₂OH) by treating the free alcohol with the appropriate chlorosulfonate or sulfonate anhydride (e.g.: CH₃SO₂Cl or CF₃SO₂OSO₂CF₃) in the presence of base (e.g.: NEt₃) or in a basic organic solvent (e.g.: pyridine) and then treating the corresponding sulfonate with trimehylsulfoxonium iodide (Me₃S(O)I) and a strong base (e.g.: NaH) in an aprotic polar solvent (e.g.; dimethylsulfoxide) at room temperature.

Salts, Solvates, Stereoisomers, Derivatives, Prodrugs and Active Metabolites of the Novel Compounds of the Invention

The present invention further encompasses salts, solvates, stereoisomers, prodrugs and active metabolites of the compounds of formula I.

The term “salts” can include acid addition salts or addition salts of free bases. Preferably, the salts are pharmaceutically acceptable. Examples of acids which may be employed to form pharmaceutically acceptable acid addition salts include, but are not limited to, salts derived from nontoxic inorganic acids such as nitric, phosphoric, sulfuric, or hydrobromic, hydroiodic, hydrofluoric, phosphorous, as well as salts derived from nontoxic organic acids such as aliphatic mono- and dicarboxylic acids, phenyl-substituted alkanoic acids, hydroxyl alkanoic acids, alkanedioic acids, aromatic acids, aliphatic and aromatic sulfonic acids, and acetic, maleic, succinic, or citric acids. Non-limiting examples of such salts include napadisylate, besylate, sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge, et al. “Pharmaceutical Salts,” J. Pharma. Sci. 1977; 66:1).

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). Preferably, as used herein, the term “pharmaceutically acceptable” means approved by a regulatory agency of the federal or a state government or listed in the U.S. Pharmacopoeia or other generally recognized pharmacopeias for use in mammals, and more particularly in humans.

Typically, a pharmaceutically acceptable salt of a compound of formula I may be readily prepared by using a desired acid or base as appropriate. The salt may precipitate from solution and be collected by filtration or may be recovered by evaporation of the solvent. For example, an aqueous solution of an acid such as hydrochloric acid may be added to an aqueous suspension of a compound of formula I and the resulting mixture evaporated to dryness (lyophilized) to obtain the acid addition salt as a solid. Alternatively, a compound of formula I may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

The acid addition salts of the compounds of formula I may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine.

The base addition salts of said acidic compounds are prepared by contacting the free acid form with a sufficient amount of the desired base to produce the salt in the conventional manner. The free acid form may be regenerated by contacting the salt form with an acid and isolating the free acid.

Compounds of the invention may have both a basic and an acidic center may and therefore be in the form of zwitterions.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention. The salts of the compound of formula I may form solvates (e.g., hydrates) and the invention also includes all such solvates. The meaning of the word “solvates” is well known to those skilled in the art as a compound formed by interaction of a solvent and a solute (i.e., solvation). Techniques for the preparation of solvates are well established in the art (see, for example, Brittain. Polymorphism in Pharmaceutical solids. Marcel Decker, New York, 1999.).

The compounds of formula I have one or more chirality centers and, depending on the nature of individual substituents, they can also have geometrical isomers. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has a chiral center, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomer respectively). A chiral compound can exist as either an individual enantiomer or as a mixture of enantiomers. A mixture containing equal proportions of the enantiomers is called a “racemic mixture”. A mixture containing unequal portions of the enantiomers is described as having an “enantiomeric excess” (ee) of either the R or S compound. The excess of one enantiomer in a mixture is often described with a % enantiomeric excess (% ee) value determined by the formula:
% ee=(R)−(S)/(R)+(S)

The ratio of enantiomers can also be defined by “optical purity” wherein the degree at which the mixture of enantiomers rotates plane polarized light is compared to the individual optically pure R and S compounds. Optical purity can be determined using the following formula:
Optical purity=enant.ₘₐⱼₒᵣ/(enant.ₘₐⱼₒᵣ+enant.ₘᵢₙₒᵣ)

The present invention encompasses all individual isomers of compounds of formula I. The description or naming of a particular compound in the specification and claims is intended to include both individual enantiomers and mixtures, racemic or otherwise, thereof. Methods for the determination of stereochemistry and the resolution of stereoisomers are well-known in the art.

The present invention also encompasses stereoisomers of the syn-anti type, and mixtures thereof encountered when an oxime or similar group is present. The group of highest Cahn-Ingold-Prelog priority attached to one of the terminal doubly bonded atoms of the oxime, is compared with hydroxyl group of the oxime. The stereoisomer is designated as Z (zusammen=together) or Syn if the oxime hydroxyl lies on the same side of a reference plane passing through the C═N double bond as the group of highest priority; the other stereoisomer is designated as E (entgegen=opposite) or Anti.

It will be appreciated by those skilled in the art that it may be desirable to use protected derivatives of intermediates used in the preparation of the compounds of formula I. Protection and deprotection of functional groups may be performed by methods known in the art (see, for example, Green and Wuts Protective Groups in Organic Synthesis. John Wiley and Sons, New York, 1999.). Hydroxyl or amino groups may be protected with any hydroxyl or amino protecting group. The amino protecting groups may be removed by conventional techniques. For example, acyl groups, such as alkanoyl, alkoxycarbonyl and aroyl groups, may be removed by solvolysis, e.g., by hydrolysis under acidic or basic conditions. Arylmethoxycarbonyl groups (e.g., benzyloxycarbonyl) may be cleaved by hydrogenolysis in the presence of a catalyst such as palladium-on-charcoal.

The present invention also encompasses prodrugs of the compounds of formula I, i.e., compounds which release an active parent drug according to formula I in vivo when administered to a mammalian subject. A prodrug is a pharmacologically active or more typically an inactive compound that is converted into a pharmacologically active agent by a metabolic transformation. Prodrugs of a compound of formula I are prepared by modifying functional groups present in the compound of formula I in such a way that the modifications may be cleaved in vivo to release the parent compound. In vivo, a prodrug readily undergoes chemical changes under physiological conditions (e.g., are acted on by naturally occurring enzyme(s)) resulting in liberation of the pharmacologically active agent. Prodrugs include compounds of formula I wherein a hydroxy, amino, or carboxy group of a formula I compound is bonded to any group that may be cleaved in vivo to regenerate the free hydroxyl, amino or carboxy group, respectively. Examples of prodrugs include, but are not limited to esters (e.g., acetate, formate, and benzoate derivatives) of compounds of formula I or any other derivative which upon being brought to the physiological pH or through enzyme action is converted to the active parent drug. Conventional procedures for the selection and preparation of suitable prodrug derivatives are described in the art (see, for example, Bundgaard. Design of Prodrugs. Elsevier, 1985).

Prodrugs may be administered in the same manner as the active ingredient to which they convert or they may be delivered in a reservoir form, e.g., a transdermal patch or other reservoir which is adapted to permit (by provision of an enzyme or other appropriate reagent) conversion of a prodrug to the active ingredient slowly over time, and delivery of the active ingredient to the patient.

Unless specifically indicated, the term “active ingredient” is to be understood as referring to a compound of formula I as defined herein.

The present invention also encompasses metabolites. “Metabolite” of a compound disclosed herein is a derivative of a compound which is formed when the compound is metabolised. The term “active metabolite” refers to a biologically active derivative of a compound which is formed when the compound is metabolised. The term “metabolised” refers to the sum of the processes by which a particular substance is changed in the living body. In brief, all compounds present in the body are manipulated by enzymes within the body in order to derive energy and/or to remove them from the body. Specific enzymes produce specific structural alterations to the compound. For example, cytochrome P450 catalyses a variety of oxidative and reductive reactions while uridine diphosphate glucuronyltransferases catalyse the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996), pages 11-17.

Metabolites of the compounds disclosed herein can be identified either by administration of compounds to a host and analysis of tissue samples from the host, or by incubation of compounds with hepatic cells in vitro and analysis of the resulting compounds. Both methods are well known in the art.

The Novel Compounds of the Invention are Aldosterone Receptor Antagonists

The novel compounds of formula I are antagonists of the aldosterone receptor (MR), a member of the steroid receptor superfamily of intracellular receptors.

The term “steroid receptor” refers to the subset of intracellular receptors comprising the aldosterone receptor (also known as the mineralocorticoid receptor, MR), progesterone receptor (PR), androgen receptor (AR), estrogen receptor (ER), and glucocorticoid receptor (GR). These steroid receptor superfamily members share a conserved protein structure consisting of a variable N-terminal domain often exhibiting a constitutive transactivation function, a conserved zinc-finger type DNA binding domain, a variable linker region, and a multifunctional C-terminal domain responsible for ligand binding, dimerization, and ligand-regulated transactivation. Steroid receptors are classified as MR, PR, AR, ER, or GR based upon primary amino acid sequence, DNA binding domain target sequence binding specificity, ligand binding specificity, target gene(s) specificity, and pharmacological effects.

The terms “aldosterone receptor” and “mineralocorticoid receptor” are used interchangeably herein and refer to any member of the mineralocorticoid receptor subfamily of the steroid receptor superfamily of intracellular receptors. The nucleotide and amino acid sequences for mineralocorticoid receptor orthologs from a variety of species (including human, chimpanzee, mouse, rat, sheep, chicken, rainbow trout, and Xenopus) are known in the art. The mineralocorticoid receptor subfamily proteins preferentially bind to the hormone ligands aldosterone and 11-deoxycorticosterone. In preferred embodiments, the mineralocorticoid receptor is a mammalian mineralocorticoid receptor. Generally speaking, the mammalian mineralocorticoid receptors have at least 80% amino acid sequence identity to each other. In particularly preferred embodiments, the mineralocorticoid receptor (MR) is a human mineralocorticoid receptor (hMR). Exemplary nucleotide and amino acid sequences for hMR are set forth in SEQ ID NOs 1 and 2, respectively.

The term “progesterone receptor” refers to any member of the progesterone receptor subfamily of the steroid receptor superfamily of intracellular receptors. The nucleotide and amino acid sequences for progesterone receptor orthologs from a variety of species (including human, mouse, rat, sheep, chicken, eel, alligator, and Xenopus) are known in the art. The progesterone receptor subfamily proteins preferentially bind to the hormone ligands pregnenolone and progesterone. In preferred embodiments, the progesterone receptor is a mammalian progesterone receptor. Generally speaking, the mammal progesterone receptors have at least 80% amino acid sequence identity to each other. In particularly preferred embodiments, the progesterone receptor (PR) is a human progesterone receptor (hPR). Exemplary nucleotide and amino acid sequences for hPR are set forth in SEQ ID NOs 3 and 4, respectively.

The term “androgen receptor” refers to any member of the androgen receptor subfamily of the steroid receptor superfamily of intracellular receptors. The nucleotide and amino acid sequences for androgen receptor orthologs from a variety of species (including human, chimpanzee, mouse, rat, sheep, chicken, eel, zebrafish, and Xenopus) are known in the art. The androgen receptor subfamily proteins preferentially bind to the hormone ligands testosterone and dehydroepiandrosterone. In preferred embodiments, the androgen receptor is a mammalian androgen receptor. Generally speaking, the mammalian androgen receptors have at least 80% amino acid sequence identity to each other. In particularly preferred embodiments, the androgen receptor (AR) is a human androgen receptor (hAR). Exemplary nucleotide and amino acid sequences for hAR are set forth in SEQ ID NOs 5 and 6, respectively.

The term “estrogen receptor” refers to any member of the estrogen receptor subfamily of the steroid receptor superfamily of intracellular receptors. At least two variants of estrogen receptor have been characterized, estrogen receptor alpha (ERα) and estrogen receptor beta (ERβ). The nucleotide and amino acid sequences for estrogen receptor orthologs from a variety of species are known in the art: including those for ERα from human, mouse, rat, sheep, chicken, quail, alligator, and Xenopus, and those for ERβ from human, mouse, rat, sheep, chicken, quail, zebrafish, and Xenopus. The estrogen receptor subfamily proteins preferentially bind to the hormone ligand estradiol. In preferred embodiments, the estrogen receptor is a mammalian estrogen receptor. In particularly preferred embodiments, the mammalian estrogen receptor is a mammalian estrogen receptor alpha (ERα) or a mammalian estrogen receptor beta (ERβ). Generally speaking, the mammalian estrogen receptor alpha (ERα) receptors have at least 80% amino acid sequence identity to each other. Generally speaking, the mammalian estrogen receptor beta (ERβ) receptors have at least 80% amino acid sequence identity to each other. In particularly preferred embodiments, the estrogen receptor (ER) is a human estrogen receptor (hER). Exemplary nucleotide and amino acid sequences for hERα are set forth in SEQ ID NOs 7 and 8, respectively. Exemplary nucleotide and amino acid sequences for hERβ are set forth in SEQ ID NOs 9 and 10, respectively.

The term “glucocorticoid receptor” refers to any member of the glucocorticoid receptor subfamily of the steroid receptor superfamily of intracellular receptors. The nucleotide and amino acid sequences for glucocorticoid receptor orthologs from a variety of species (including human, chimpanzee, mouse, rat, sheep, pig, rainbow trout, cichlid fish and Xenopus) are known in the art. The glucocorticoid receptor subfamily proteins preferentially bind to the hormone ligands cortisol (a.k.a. hydrocortisone) and corticosterone. In preferred embodiments, the glucocorticoid receptor is a mammalian glucocorticoid receptor. Generally speaking, the mammalian glucocorticoid receptors have at least 80% amino acid sequence identity to each other. In particularly preferred embodiments, the glucocorticoid receptor (GR) is a human glucocorticoid receptor (hGR). Exemplary nucleotide and amino acid sequences for hGR are set forth in SEQ ID NOs X and X, respectively.

The terms “aldosterone receptor antagonist”, “antagonist of the aldosterone receptor”, “mineralocorticoid receptor antagonist”, and “antagonist of the mineralocorticoid receptor” are used interchangeably herein and refer to a compound that inhibits or decreases activity of the mineralocorticoid receptor. Without intending to be limited by mechanism, it is thought that the novel compounds of the invention are aldosterone receptor antagonists due to the ability to compete with an aldosterone receptor agonist (e.g., aldosterone) for binding to the ligand binding domain of the aldosterone receptor.

The terms “aldosterone receptor agonist”, “agonist of the aldosterone receptor”, “mineralocorticoid receptor agonist”, and “agonist of the mineralocorticoid receptor” are used interchangeably herein and refer to a compound that increases or enhances activity of the mineralocorticoid receptor. For example, aldosterone, cortisol and deoxycortisone acetate (DOCA) are aldosterone receptor agonists.

The ability of the novel compounds of the invention to act as aldosterone receptor antagonists may be assessed by any of the means well-established in the art.

For example, in vitro and in vivo competitive binding assays for determination of the affinity of test compounds for binding to the aldosterone receptor, and of the ability of test compounds to inhibit binding of ligand (e.g., aldosterone) to the aldosterone receptor, have been described (see, for example, U.S. Pat. No. 4,081,538; U.S. Pat. No. 4,603,128; Fanestil and Edelman. Proc Natl Acad Sci USA 1966; 56:872-879; Herman, et al. J Biol Chem 1968; 243:3849-3856; Funder, et al. Biochem Pharmacol 1974; 23:1493-1501; Marver, et al. Proc Natl Acad Sci USA 1974; 71:1431-1435; Rafestin-Oblin, et al. J Steroid Biochem 1986; 25:527-534; Gasparo, et al. J Pharm Exp Therap 1987; 240:650-656; Govindan, et al. J Steroid Biochem Mol Biol 1991; 39:91-103; Claire, et al. J Med Chem 1993; 36:2404-2407; and Grob, et al. Helvetica Chimica ACTA 1997; 80:566-585).

For example, the ability of a test compound to compete with aldosterone for in vitro binding to kidney slices isolated from adrenalectomized rats may be assessed (see, for example, U.S. Pat. No. 4,081,538; Marver, et al. Proc Natl Acad Sci USA 1974; 71:1431-1435; and Funder, et al. Biochem Pharmacol 1974; 23:1493-1501). For example, the ability of a test compound to compete with aldosterone for binding to supernatants of kidney cell extracts isolated from adrenalectomized rats or rabbits may be assessed (see, for example, Herman, et al. J Biol Chem 1968; 243:3849-3856; Rafestin-Oblin, et al. J Steroid Biochem 1986; 25:527-534; Gasparo, et al. J Pharm Exp Therap 1987; 240:650-656; and Claire, et al. J Med Chem 1993; 36:2404-2407). For example, the ability of a test compound to compete with aldosterone for binding to cell-free extracts prepared from in vitro cultured cells ectopically expressing MR may be assessed (see, for example, Govindan, et al. J Steroid Biochem Mol Biol 1991; 39:91-103; Alnemri, et al. J Biol Chem 1991; 266:18072-18081; and in the EXAMPLES, below). For example, a test compound and aldosterone may be injected into rats, and the ability of the test compound to reduce the levels of protein-bound aldosterone subsequently detected in the kidney may be assessed (see, for example, U.S. Pat. No. 4,081,538; Fanestil and Edelman. Proc Natl Acad Sci USA 1966; 56:872-879; Herman, et al. J Biol Chem 1968; 243:3849-3856; Marver, et al. Proc Natl Acad Sci USA 1974; 71:1431-1435; and Gasparo, et al. J Pharm Exp Therap 1987; 240:650-656).

In another example, the ability of a test compound to inhibit agonist-induced (e.g., aldosterone-induced) expression of a gene whose expression is regulated by the mineralocorticoid receptor may be assessed. For example, in vitro cultured cell lines expressing MR and containing a reporter gene in which expression of chloramphenicol acetyltransferase (CAT) is regulated by the Mouse Mammary Tumour Virus Long Terminal Repeat (MMTV LTR) have been described (see, for example, Govindan, et al. J Steroid Biochem Mol Biol 1991; 39:91-103 and Couette, et al. Biochim Biophys Acta 1994; 1219:607-612). In such cells, treatment with an aldosterone receptor agonist (e.g., aldosterone) induces CAT reporter gene expression. Such cell lines may be used in assays wherein the ability of a test compound to inhibit aldosterone receptor agonist-induced (e.g., aldosterone-induced) MMTV LTR-CAT reporter gene expression is quantitated.

For example, in vitro cultured cell lines expressing a fusion protein of the Saccharomyces cerevisiae GAL4 protein DNA binding domain (GAL4 DBD) and the MR ligand binding domain (MR LBD), and containing a reporter gene in which expression of luciferase is regulated by a GAL4 target sequence, have been described (see, for example, Jausons-Loffreda, et al. J Steroid Biochem Molec Biol 1994; 49:31-38). For example, in vitro cultured human embryonic kidney (HEK293T) cells expressing a fusion protein of the Saccharomyces cerevisiae GAL4 protein DNA binding domain (GAL4 DBD) and the MR ligand binding domain (MR LBD), and containing a reporter gene in which expression of beta lactamase is regulated by a GAL4 target sequence, are commercially available (Invitrogen catalogue number K1071). In such cells, treatment with an aldosterone receptor agonist (e.g., aldosterone) induces reporter gene expression. Such cell lines have been used in assays wherein the ability of a test compound to inhibit aldosterone receptor agonist-induced (e.g., aldosterone-induced) reporter gene expression is quantitated.

In vivo assays for aldosterone receptor antagonist activity are well-established in a variety of animal models, including rats (see, for example, U.S. Pat. No. 4,081,538; U.S. Pat. No. 4,180,505; U.S. Pat. No. 4,218,446; U.S. Pat. No. 4,328,221; U.S. Pat. No. 4,603,128; Kagawa, et al. J Pharmacol Exp Ther 1959; 126:123-130; Marver, et al. Proc Natl Acad Sci USA 1974; 71:1431-1435; Casals-Stenzel, et al. Eur J Pharmacol 1982; 80:37-54; Losert, et al. Arzneimittelforschung 1986; 36:1582-1600; Gasparo, et al. J Pharm Exp Therap 1987; 240:650-656; Kalimi, et al. Am J Physiol 1990; 258:E737-739; Opoku, et al. Am J Physiol 1991;260:E269-E271; Sekihara and Yazaki. J Steroid Biochem Mol Biol 1993; 45:235-238; and Grob, et al. Helvetica Chimica ACTA 1997; 80:566-585), sheep (see, for example, Kairaitis and Lumbers. J Dev Physiol 1990; 13:347-351 and Rabinowitz, et al. Am J Physiol 1985; 249:R455-461), rabbits (see, for example, Mihailidou, et al. Circulation Research 2000;86: 37-42), rhesus monkeys (see, for example, Hofmann, et al. J Pharmacol Exp Ther 1977; 202:216-220), and humans (see, for example, Ramsey, et al. Br J Clin Pharmacol 1975; 2:271-276; Ramsey, et al. Clin Pharmacol Ther 1975; 18:391-400; Ramsey, et al. Clin Pharmacol Ther 1976; 20:167-177; Levine, et al. Eur J Clin Pharmacol 1976; 09:381-386; Casals-Stenzel, et al. Eur J Clin Pharmacol 1977; 12:247-255; McInnes, et al. Methods Find Exp Clin Pharmacol 1982; 4:49-71; and Ulmann, et al. Eur J Clin Pharm 1985; 28:531-535).

For example, in an assay commonly referred to as the Kagawa assay, a test compound may be evaluated in adrenalectomized rats for the ability to inhibit aldosterone receptor agonist-induced [e.g., aldosterone or deoxycortisone acetate (DOCA)-induced] reduction in the urinary Na⁺/K⁺ ratio (see, for example, U.S. Pat. No. 4,081,538; U.S. Pat. No. 4,180,505; U.S. Pat. No. 4,218,446; U.S. Pat. No. 4,328,221; U.S. Pat. No. 4,603,128; Kagawa, et al. J Pharmacol Exp Ther 1959; 126:123-130; Marver, et al. Proc Natl Acad Sci USA 1974; 71:1431-1435; Casals-Stenzel, et al. Eur J Pharmacol 1982; 80:37-54; Losert, et al. Arzneimittelforschung 1986; 36:1582-1600; Gasparo, et al. J Pharm Exp Therap 1987; 240:650-656; Sekihara and Yazaki J Steroid Biochem Mol Biol 1993; 45:235-238; and Grob, et al. Helvetica Chimica ACTA 1997; 80:566-585).

Other in vivo assays for aldosterone receptor antagonist activity include assays for the ability of a test compound to: inhibit aldosterone receptor agonist-induced alterations in sodium retention in adrenalectomized rats (see, for example, U.S. Pat. No. 4,328,221); inhibit aldosterone receptor agonist-induced, alterations in blood pressure in rats (see, for example, Kalimi, et al. Am J Physiol 1990; 258:E737-739 and Opoku, et al. Am J Physiol 1991;260:E269-E271); inhibit aldosterone receptor agonist-induced alterations in urinary Na⁺/K⁺ ratio in sheep (see, for example, Kairaitis and Lumbers. J Dev Physiol 1990; 13:347-351); inhibit aldosterone receptor agonist-induced alterations in urinary K⁺ excretion in sheep (see, for example, Rabinowitz, et al. Am J Physiol 1985; 249:R455-461); inhibit aldosterone receptor agonist-induced alterations in urinary Na⁺/K⁺ ratio in rhesus monkey (see, for example, Hofmann, et al. J Pharmacol Exp Ther 1977; 202:216-220); and inhibit aldosterone receptor agonist-induced alterations in myocardial Na⁺—K⁺ pump function in rabbits (see, for example, Mihailidou, et al. Circulation Research 2000; 86:37-42).

In vivo assays for the evaluation of aldosterone receptor antagonists in human clinical models have also been described (see, for example, Ramsey, et al. Br J Clin Pharmacol 1975; 2:271-276; Ramsey, et al. Clin Pharmacol Ther 1975; 18:391-400; Ramsey, et al. Clin Pharmacol Ther 1976; 20:167-177; Levine, et al. Eur J Clin Pharmacol 1976; 09:381-386; Casals-Stenzel, et al. Eur J Clin Pharmacol 1977; 12:247-255; McInnes, et al. Methods Find Exp Clin Pharmacol 1982; 4:49-71; and Ulmann, et al. Eur J Clin Pharm 1985; 28:531-535). For example, a test compound may be evaluated in humans for the ability to counteract hypermineralocorticism induced by administration of 9α-fluorohydrocortisone (9α-FHC, also known as fludrocortisone) (see, for example, Ramsey, et al. Br J Clin Pharmacol 1975; 2:271-276; Ramsey, et al. Clin Pharmacol Ther 1975; 18:391-400; Ramsey, et al. Clin Pharmacol Ther 1976; 20:167-177; and Ulmann, et al. Eur J Clin Pharm 1985; 28:531-535), exogenous aldosterone (see, for example, Casals-Stenzel, et al. Eur J Clin Pharmacol 1977; 12:247-255 and Ulmann, et al. Eur J Clin Pharm 1985; 28:531-535) or furosemide (also known as frusemide) (see, for example, Levine, et al. Eur J Clin Pharmacol 1976; 09:381-386 and Ulmann, et al. Eur J Clin Pharm 1985; 28:531-535).

Aldosterone receptor ligands for use in the above described assays, including aldosterone and DOCA, are available, in both radiolabelled and non-labelled forms, from a variety of commercial sources, including Du Pont-New England Nuclear, Amersham, Sigma-Aldrich, and Perkin Elmer.

In any assay to assess the activity of a novel compound as an aldosterone receptor antagonist, the potency of the novel antagonist may be quantitated as an IC₅₀ value, where “IC₅₀” is defined as the dose of compound effective to yield a 50% reduction in aldosterone receptor agonist activity under the conditions of the given assay. For example, in the case of a competitive binding assay, the IC₅₀ of a test compound may be calculated as the concentration of test compound effective to reduce binding of an aldosterone receptor agonist to the aldosterone receptor by 50%. For example, in the case of an assay for aldosterone receptor-dependent reporter gene expression in an in vitro cultured cell line, the IC₅₀ of a test compound may be calculated as the concentration of test compound effective to reduce aldosterone receptor agonist-induced reporter gene expression by 50%. In another example, in the case of the in vivo Kagawa assay, the IC₅₀ of a test compound may be calculated as the dose of test compound effective to reduce the aldosterone receptor agonist-induced suppression of urinary Na⁺/K⁺ ratio by 50%.

Generally speaking, when tested in an in vitro competitive binding assay, the novel compounds of the invention show an IC₅₀ of ≦100 μM. Preferred compounds of the invention show an IC₅₀ of ≦4000 μM when tested in an in vitro competitive binding assay. Particularly preferred compounds of the invention show an IC₅₀ of ≦200 μM when tested in an in vitro competitive binding assay.

Generally speaking, when tested in an assay for aldosterone receptor-dependent reporter gene expression in an in vitro cultured cell line, the novel compounds of the invention show an IC₅₀ of ≦10 μM. Preferred compounds of the invention show an IC₅₀ of ≦5000 μM tested in an assay for aldosterone receptor-dependent reporter gene expression in an in vitro cultured cell line. Particularly preferred compounds of the invention show an IC₅₀ of ≦200 μM tested in an assay for aldosterone receptor-dependent reporter gene expression in an in vitro cultured cell line.

In preferred embodiments, the activity of a novel compound of the invention to act as an aldosterone receptor antagonist is assessed by any of the means well-established in the art and compared to the activity of the known aldosterone receptor antagonists spironolactone and/or eplerenone in the same assay.

To provide a measure of the potency of a test compound as an antagonist of MR, the IC₅₀ of a test compound (IC₅₀^{Test}) calculated for the defined conditions of the given assay and the IC₅₀ of the known MR antagonists eplerenone (IC₅₀^{Ep}) or spironolactone (IC₅₀^{Sp}) calculated under the same conditions in the same assay may be used to calculate an IC₅₀ ratio (RIC₅₀) according to the following equations:
RIC₅₀^{Ep/Test} =IC₅₀^{Ep}/IC₅₀^{Test} and
RIC₅₀^{SP/Test}=IC₅₀^{Sp}/IC₅₀^{Test}

In preferred embodiments the assay is an in vitro competitive binding assay and the IC₅₀ of a test compound (IC₅₀^{Test}) for ligand binding to MR and the IC₅₀ of the known MR antagonists eplerenone (IC₅₀^{Ep}) and/or spironolactone (IC₅₀^{Sp}) for ligand binding to MR are used to calculate an IC₅₀ ratio (RIC₅₀).

In another preferred embodiment, the assay is an assay for aldosterone receptor-dependent reporter gene expression in an in vitro cultured cell line and the IC₅₀ of a test compound (IC₅₀^{Test}) for aldosterone receptor agonist-induced reporter gene expression and the IC₅₀ of the known MR antagonists eplerenone (IC₅₀^{Ep}) and/or spironolactone (IC₅₀^{Sp}) for aldosterone receptor agonist-induced reporter gene expression are used to calculate an IC₅₀ ratio (RIC₅₀).

In one embodiment, preferred compounds of the invention show an IC₅₀^{Test} that is at least comparable to IC₅₀^{Ep} (i.e. RIC₅₀^{Ep/Test}≧0.5). More preferred compounds of the invention show an IC₅₀^{Test} that is lower than IC₅₀^{Ep} (i.e. RIC₅₀^{EP/Test}>1.0). Particularly preferred compounds of the invention show an IC₅₀^{Test} that is an order of magnitude lower than IC₅₀^{Ep} (i.e. RIC₅₀^{EP/Test}≧10.0).

In another embodiment, preferred compounds of the invention show an IC₅₀ Test that is comparable to IC₅₀^{Sp} (i.e. RIC₅₀^{Sp/Test}≧0.5). Particularly preferred compounds of the invention show an IC₅₀^{Test} that is lower than IC₅₀^{Sp} (i.e. RIC₅₀^{SP/Test}>1.0).

In preferred embodiments, compounds of the invention show an IC₅₀^{Test} that is at least comparable to IC₅₀^{Ep} and at least comparable to IC₅₀^{Sp} (i.e. RIC₅₀^{Ep/Test}≧0.5 and RIC₅₀^{Sp/Test}≧0.5). Preferred compounds of the invention show an IC₅₀^{Test} that is lower than IC₅₀^{Ep} and comparable to IC₅₀^{Sp} (i.e. RIC₅₀^{Ep/Test}>1 and RIC₅₀^{Sp/Test}≧0.5). Particularly preferred compounds of the invention show an IC₅₀^{Test} that is an order of magnitude lower than IC₅₀^{Ep} and lower than IC₅₀^{Sp} (i.e. RIC₅₀^{EP/Test}≧10.0 and RIC₅₀^{Sp/Test}>1).

In preferred embodiments, the compounds of the invention are not themselves aldosterone receptor agonists. The ability of the novel compounds of the invention to act as aldosterone receptor agonists may be assessed by any of the means well-established in the art.

For instance, the ability of a test compound to induce expression of a gene whose expression is regulated by the mineralocorticoid receptor may be assessed. For example, in vitro cultured cell lines expressing MR and containing a reporter gene in which expression of chloramphenicol acetyltransferase (CAT) is regulated by the Mouse Mammary Tumour Virus Long Terminal Repeat (MMTV LTR) have been described (see, for example, Govindan, et al. J Steroid Biochem Mol Biol 1991; 39:91-103 and Couette, et al. Biochim Biophys Acta 1994; 1219:607-612). In such cells, treatment with an aldosterone receptor agonist (e.g., aldosterone) induces CAT reporter gene expression. Such cell lines may be used in assays wherein the ability of a test compound to induce MMTV LTR-CAT reporter gene expression is quantitated.

For example, in vitro cultured cell lines expressing a fusion protein of the Saccharomyces cerevisiae GAL4 protein DNA binding domain and the MR ligand binding domain, and containing a reporter gene in which expression of luciferase is regulated by a GAL4 target sequence, have been described (see, for example, Jausons-Loffreda, et al. J Steroid Biochem Molec Biol 1994; 49:31-38). In vitro cultured human embryonic kidney (HEK293T) cells expressing a fusion protein of the Saccharomyces cerevisiae GAL4 protein DNA binding domain (GAL4 DBD) and the MR ligand binding domain (MR LBD), and containing a reporter gene in which expression of beta lactamase is regulated by a GAL4 target sequence, which cell lines are commercially available (Invitrogen catalogue number K1071). In such cells, treatment with an aldosterone receptor agonist (e.g., aldosterone) induces reporter gene expression. Such cell lines may be used in assays wherein the ability of a test compound to induce reporter gene expression is quantitated.

In vivo assays for aldosterone receptor agonist activity are well-established in a variety of animal models, including rats (see, for example, U.S. Pat. No. 4,081,538; U.S. Pat. No. 4,180,505; U.S. Pat. No. 4,218,446; U.S. Pat. No. 4,328,221; U.S. Pat. No. 4,603,128; Kagawa, et al. J Pharmacol Exp Ther 1959; 126:123-130; Marver, et al. Proc Natl Acad Sci USA 1974; 71:1431-1435; Casals-Stenzel, et al. Eur J Pharmacol 1982; 80:37-54; Losert, et al. Arzneimittelforschung 1986; 36:1582-1600; Gasparo, et al. J Pharm Exp Therap 1987; 240:650-656; Kalimi, et al. Am J Physiol 1990; 258:E737-739; Opoku, et al. Am J Physiol 1991; 260:E269-E271; Sekihara and Yazaki J Steroid Biochem Mol Biol 1993; 45:235-238; and

Grob, et al. Helvetica Chimica ACTA 1997; 80:566-585), sheep (see, for example, Kairaitis and Lumbers. J Dev Physiol 1990; 13:347-351 and Rabinowitz, et al. Am J Physiol 1985; 249:R455-461), rabbits (see, for example, Mihailidou, et al. Circulation Research 2000; 86:37-42), rhesus monkeys (see, for example, Hofmann, et al. J Pharmacol Exp Ther 1977; 202:216-220), and humans (see, for example, Ramsey, et al. Br J Clin Pharmacol 1975; 2:271-276; Ramsey, et al. Clin Pharmacol Ther 1975; 18:391-400; Ramsey, et al. Clin Pharmacol Ther 1976; 20:167-177; Levine, et al. Eur J Clin Pharmacol 1976; 09:381-386; Casals-Stenzel, et al. Eur J Clin Pharmacol 1977; 12:247-255; McInnes, et al. Methods Find Exp Clin Pharmacol 1982; 4:49-71; and Ulmann, et al. Eur J Clin Pharm 1985; 28:531-535).

For example, in an assay commonly referred to as the Kagawa assay, a test compound may be evaluated in adrenalectomized rats for the ability to reduce the urinary Na⁺/K⁺ ratio (see, for example, U.S. Pat. No. 4,081,538; U.S. Pat. No. 4,180,505; U.S. Pat. No. 4,218,446; U.S. Pat. No. 4,328,221; U.S. Pat. No. 4,603,128; Kagawa, et al. J Pharmacol Exp Ther 1959; 126:123-130; Marver, et al. Proc Natl Acad Sci USA 1974; 71:1431-1435; Casals-Stenzel, et al. Eur J Pharmacol 1982; 80:37-54; Losert, et al. Arzneimittelforschung 1986; 36:1582-1600; Gasparo, et al. J Pharm Exp Therap 1987; 240:650-656; Sekihara and Yazaki J Steroid Biochem Mol Biol 1993; 45:235-238; and Grob, et al. Helvetica Chimica ACTA 1997; 80:566-585).

Other in vivo assays include assays for the ability of a test compound to: alter sodium retention in adrenalectomized rats (see, for example, U.S. Pat. No. 4,328,221); increase blood pressure in rats (see, for example, Kalimi, et al. Am J Physiol 1990; 258:E737-739 and Opoku, et al. Am J Physiol 1991; 260:E269-E271); reduce urinary Na⁺/K⁺ ratio in sheep (see, for example, Kairaitis and Lumbers. J Dev Physiol 1990; 13:347-351); alter urinary K⁺ excretion in sheep (see, for example, Rabinowitz, et al. Am J Physiol 1985; 249:R455461); reduce urinary Na⁺/K⁺ ratio in rhesus monkey (see, for example, Hofmann, et al. J Pharmacol Exp Ther 1977; 202:216-220); and alter myocardial Na⁺—K⁺ pump function in rabbits (see, for example, Mihailidou, et al. Circulation Research 2000; 86:37-42).

In vivo assays for the evaluation of aldosterone receptor agonists in human clinical models have also been described (see, for example, Ramsey, et al. Br J Clin Pharmacol 1975; 2:271-276; Ramsey, et al. Clin Pharmacol Ther 1975; 18:391-400; Ramsey, et al. Clin Pharmacol Ther 1976; 20:167-177; Levine, et al. Eur J Clin Pharmacol 1976; 09:381-386; Casals-Stenzel, et al. Eur J Clin Pharmacol 1977; 12:247-255; McInnes, et al. Methods Find Exp Clin Pharmacol 1982; 4:49-71; and Ulmann, et al. Eur J Clin Pharm 1985; 28:531-535).

Aldosterone receptor ligands for use in the above described assays, including aldosterone and DOCA, are available, in both radiolabelled and non-labelled forms, from a variety of commercial sources, including Du Pont-New England Nuclear, Amersham, Sigma-Aldrich, and Perkin Elmer.

Generally speaking, the novel compounds of the invention are at least three orders of magnitude less potent as aldosterone receptor agonists than as aldosterone receptor antagonists (i.e., a one thousand fold higher concentration of the compound is required to produce an agonist effect in a given assay versus the concentration of the compound required to produce an antagonist effect in an equivalent assay). For example, in the case of an assay for aldosterone receptor-dependent reporter gene expression in an in vitro cultured cell line, the concentration of the novel compound effective to reduce aldosterone receptor agonist-induced reporter gene expression by 50% (i.e., the IC₅₀) is at least three orders of magnitude less than the concentration of novel compound effective to induce a level of reporter gene expression that is 50% of that observed in the absence of test compound in the antagonist assay (i.e., 50% of the observed level of aldosterone receptor agonist-induced reporter gene expression).

In preferred embodiments, the results of a given assay to assess the activity of a novel compound as an aldosterone receptor agonist performed under defined conditions may be compared to those obtained for a known aldosterone receptor agonist in the same assay performed under the same conditions. In preferred embodiments, the known agonist is aldosterone. In preferred embodiments, the novel compounds of the invention are at least three orders of magnitude less potent than aldosterone as an aldosterone receptor agonist. In more preferred embodiments, the novel compounds of the invention are at least four orders of magnitude less potent than aldosterone as an aldosterone receptor agonist. In particularly preferred embodiments, the novel compounds of the invention are at least five orders of magnitude less potent than aldosterone as an aldosterone receptor agonist.

The novel compounds of the invention are selective aldosterone receptor antagonists. The terms “selective aldosterone receptor antagonist”, “selective antagonist of the aldosterone receptor”, “selective mineralocorticoid receptor antagonist”, and “selective antagonist of the mineralocorticoid receptor” are used interchangeably herein and refer to a compound that is more potent as an aldosterone receptor ligand than as an ER, PR, GR, and/or AR ligand (i.e., a higher concentration of the compound is required to produce a given ER, PR, GR, and/or AR receptor effect in a given assay versus the concentration of the compound required to produce an equivalent MR effect in a related assay). In preferred embodiments, a “selective aldosterone receptor antagonist” is at least one order of magnitude more potent at the MR than at the ER, PR, GR, and/or AR. In particularly preferred embodiments, a “selective aldosterone receptor antagonist” is at least two order of magnitude more potent at the MR than at the ER, PR, GR, and/or AR.

Assays to determine the ability of a test compound to act as ligand for GR, PR, AR, and/or ER are well-established in the art. For example, in vivo and in vitro assays for the ability of a test compound to act as a ligand for the GR based upon determination of tyrosine-amino-transferase (TAT) enzyme activity, mRNA levels, and/or protein levels are well-known in the art (see, for example, Campen and Fanestil. Clin Exp Hypertens 1982; 4:1627-1636 and Jausons-Loffreda, et al. J Steroid Biochem Molec Biol 1994; 49:31-38). For example, in vivo assays for the ability of a test compound to act as a ligand for the PR based upon measurement of the degree of endometrial proliferation in rabbits (commonly referred to as the Clauberg-McPhail test) are well-known in the art (see, for example U.S. Pat. No. 4,328,221). For example, in vivo assays for the ability of a test compound to act as a ligand for of the AR based upon the determination of target organ weights in rodent models are well-known in the art (see, for example, U.S. Pat. No. 4,180,505; U.S. Pat. No. 4,218,446; U.S. Pat. No. 4,328,221; and Gasparo, et al. J Pharm Exp Therap 1987; 240:650-656). For example, in vivo assays for the ability of a test compound to act as a ligand for the ER based upon the determination of target organ weights, uterine histology, and/or ovulatory cycle disruptions in rabbit and/or rodent models are well-known in the art (see, for example, U.S. Pat. No. 4,218,446; U.S. Pat. No. 4,328,221; and Gasparo, et al. J Pharm Exp Therap 1987; 240:650-656). Fluorescence polarization-based assay kits for the characterization of AR, ER, GR, or PR ligands are commercially available (e.g., from Invitrogen, Carlsbaad, Calif.).

The ability of the novel compounds of the invention to act as selective aldosterone receptor antagonists may be assessed by any of the means well-established in the art, including in vitro and in vivo assays.

For example, in vitro and in vivo competitive binding assays for determination of the binding affinity of test compounds for the aldosterone receptor versus the AR, PR, ER, and/or GR, and of the ability of test compounds to inhibit ligand (e.g., aldosterone) binding to the aldosterone receptor versus to inhibit ligand binding to AR, PR, ER, and/or GR have been described (see, for example, U.S. Pat. No. 4,603,128; Claire, et al. Endocrinology 1979; 104:1194-1200; Losert, et al. Arzneimittelforschung 1986; 36: 1582-1600; Rafestin-Oblin, et al. J Steroid Biochem 1986; 25:527-534; Gasparo, et al. J Pharm Exp Therap 1987; 240:650-656; Claire, et al. J Med Chem 1993; 36:2404-2407; Fuhrmann, et al. Contraception 1996; 54:243-251; and Grob, et al Helvetica Chimica ACTA 1997; 80:566-585). Recombinant AR, ER, GR, and PR proteins that may be used in in vitro competitive binding assays are commercially available (e.g., from Invitrogen and Novocastra).

For example, the ability of a test compound to inhibit ligand-induced expression of a gene whose expression is regulated by the MR may be assessed and compared to its ability to activate or inhibit ligand-induced expression of a gene whose expression is regulated by GR, PR, AR, or ER.

For example, in vitro cultured cell lines containing a reporter gene in which expression of a reporter gene (e.g., chloramphenicol acetyltransferase or luciferase) is regulated by the Mouse Mammary Tumour Virus Long Terminal Repeat (MMTV LTR), and ectopically expressing MR, GR, PR, or AR and have been described (see, for example, Govindan, et al. J Steroid Biochem Mol Biol 1991; 39:91-103; Couette, et al. Biochim Biophys Acta 1994; 1219:607-612; Terouanne, et al. Mol Cell Endocrin 2000; 160:39-49; and Nirde, et al. Mol Pharmacol 2001; 59:1307-1313). In such cells, treatment with an appropriate agonist (e.g., aldosterone for cells expressing MR, dexamethasone for cells expressing GR, progesterone for cells expressing PR, or androgen for cells expressing AR) induces reporter gene expression. Such cells may be used in assays to characterize the ability of a test compound to inhibit aldosterone receptor agonist-induced reporter gene expression in cells expressing MR, and compared to its ability to modulate agonist-induced reporter gene expression in cells expressing GR, PR, or AR.

In another example, in vitro cultured cell lines expressing a fusion protein of the Saccharomyces cerevisiae GAL4 protein DNA binding domain and the ligand binding domain of MR, PR, ER or GR (GAL4 DBD-MR LBD; GAL4 DBD-PR LBD; GAL4 DBD-ER LBD; or GAL4 DBD-GR LBD) and containing a reporter gene (e.g., luciferase) whose expression is regulated by a GAL4 target sequence, have been described (see, for example, Webster, et al. Cell 1988; 54: 199-207 and Jausons-Loffreda, et al. J Steroid Biochem Molec Biol 1994; 49:31-38). In vitro cultured cells expressing a fusion protein of the Saccharomyces cerevisiae GAL4 protein DNA binding domain and the ligand binding domain of MR, AR, ER or GR (GAL4 DBD-MR LBD; GAL4 DBD-AR LBD; GAL4 DBD-ER LBD; or GAL4 DBD-GR LBD), and containing a reporter gene in which expression of beta lactamase is regulated by a GAL4 target sequence, are commercially available (Invitrogen catalogue numbers K1071, K1082 and K1090, K1091, and K1072, respectively). In such cells, treatment with the appropriate agonist (e.g., aldosterone for cells expressing GAL4 DBD-MR LBD, estradiol for cells expressing GAL4 DBD-ER LBD, dexamethasone for cells expressing GAL4 DBD-GR LBD, progesterone for cells expressing GAL4 DBD-PR LBD, or androgen for cells expressing GAL4 DBD-AR LBD) induces reporter gene expression. Such cells may be used in assays to characterize the ability of a test compound to inhibit aldosterone receptor agonist-induced reporter gene expression in cells expressing GAL4 DBD-MR LBD, and compared to its ability to modulate agonist-induced reporter gene expression in cells expressing GAL4 DBD-PR LBD, GAL4 DBD-ER LBD, GAL4 DBD-AR LBD, or GAL4 DBD-GR LBD.

Steroid ligands for use in the above described assays, including: aldosterone and DOCA (MR ligands), mibolerone (AR ligand), estradiol (ER ligand), dexamethasone (GR ligand), and promegestone (PR ligand) are available, in both radiolabelled and non-labelled forms, from a variety of commercial sources, including Du Pont-New England Nuclear, Amersham, Sigma-Aldrich, and Perkin Elmer.

In preferred embodiments, the activity of a novel compound of the invention to act as an aldosterone receptor antagonist is assessed by any of the means well-established in the art and compared to the activity of the novel compound to act as an AR, ER, GR, or PR antagonist in an equivalent assay under equivalent conditions. In any assay to assess the activity of a novel compound as an aldosterone receptor antagonist or an AR, ER, GR, or PR antagonist, the potency of the novel antagonist may be quantitated as an IC₅₀^{MR}, IC₅₀^{AR}, IC₅₀^{ER}, IC₅₀^{GR}, or IC₅₀^{PR} value, as applicable, where “IC₅₀” is defined as the dose of test compound effective to yield a 50% reduction in receptor agonist activity under the defined conditions of the given assay. For example, in the case of a competitive binding assay, the IC₅₀ of a test compound may be calculated as the concentration of test compound effective to reduce binding of a receptor agonist to its cognate receptor by 50%. For example, in the case of an assay for receptor-dependent reporter gene expression in an in vitro cultured cell line, the IC₅₀ of a test compound may be calculated as the concentration of test compound effective to reduce receptor agonist-induced reporter gene expression by 50%.

In preferred embodiments the assay is an in vitro competitive binding assay and the IC₅₀ of a test compound for ligand binding to MR (IC₅₀^{MR}) under given conditions, and the IC₅₀ for the test compound for ligand binding to the AR, ER, GR, and/or PR receptor (IC₅₀^{AR}, IC₅₀^{ER}, IC₅₀^{GR}, and/or IC₅₀^{PR}) under equivalent conditions are determined.

In other preferred embodiments, the assay is an assay for receptor-dependent reporter gene expression in an in vitro cultured cell line and the IC₅₀ of a test compound for aldosterone receptor agonist-dependent reporter gene expression (IC₅₀^{MR}) under given conditions in a given cell line, is determined and compared to the IC₅₀ of the test compound for AR, ER, GR, or PR receptor agonist-dependent reporter gene expression (IC₅₀^{AR}, IC₅₀^{ER}, IC₅₀^{GR}, or IC₅₀^{PR}) as determined under equivalent conditions in an equivalent cell line.

To provide a measure of the selectivity of test compounds for MR over AR, ER, GR, or PR, the IC₅₀^{AR}, IC₅₀^{ER}, IC₅₀^{GR}, or IC₅₀^{PR} of a test compound for a given assay and the IC₅₀^{MR} of the test compound for the equivalent assay may be used to calculate an inhibitory ratio (IR) according to the following equations:
IR^{AR/MR}=IC₅₀^{AR}/IC₅₀^{MR};
IR^{ER/MR}=IC₅₀^{ER}/IC₅₀^{MR};
IR^{GR/MR}=IC₅₀^{GR}/IC₅₀^{MR}; and
IR^{PR/MR}=IC₅₀^{PR}/IC₅₀^{MR};

Preferred compounds of the invention show an IC₅₀^{MR} that is lower than the IC₅₀^{AR} (i.e. IR^{AR/MR}>1.0). Preferred compounds of the invention show an IC₅₀^{MR} that is an order of magnitude lower than the IC₅₀^{AR} (i.e. IR^{AR/MR}≧10.0). Particularly preferred compounds of the invention show an IC₅₀^{MR} that is two orders of magnitude lower than the IC₅₀^{AR} (i.e. IR^{AR/MR}≧100.0).

Preferred compounds of the invention show an IC₅₀^{MR} that is lower than the IC₅₀^{ER} (i.e. IR^{ER/MR}>1.0). Preferred compounds of the invention show an IC₅₀^{MR} that is an order of magnitude lower than the IC₅₀^{ER} (i.e. IR^{ER/MR}≧10.0). Particularly preferred compounds of the invention show an IC₅₀^{MR} that is two orders of magnitude lower than the IC₅₀^{ER} (i.e. IR^{ER/MR}≧100.0). Particularly preferred compounds of the invention show an IC₅₀^{MR} that is three orders of magnitude lower than the IC₅₀^{ER} (i.e. IR^{ER/MR}≧1000.0).

Preferred compounds of the invention show an IC₅₀^{MR} that is lower than the IC₅₀^{GR} (i.e. IR^{GR/MR}≧1.0). Preferred compounds of the invention show an IC₅₀^{MR} that is an order of magnitude lower than the IC₅₀^{GR} (i.e. IR^{GR/MR}≧10.0). Particularly preferred compounds of the invention show an IC₅₀^{MR} that is two orders of magnitude lower than the IC₅₀^{GR} (i.e. IR^{GR/MR}≧100.0).

Preferred compounds of the invention show an IC₅₀^{MR} that is lower than the IC₅₀^{PR} (i.e. IR^{PR/MR}≧1.0). Preferred compounds of the invention show an IC₅₀^{MR} that is an order of magnitude lower than the IC₅₀^{PR} (i.e. IR^{PR/MR}≧10.0). Particularly preferred compounds of the invention show an IC₅₀^{MR} that is two orders of magnitude lower than the IC₅₀^{PR} (i.e. IR^{PR/MR}≧100.0). Particularly preferred compounds of the invention show an IC₅₀^{MR} that is three orders of magnitude lower than the IC₅₀^{PR} (i.e. IR^{PR/MR}≧10000).

For preferred compounds of the invention, at least two of the individual ratios selected from the group consisting of IR^{AR/MR}, IR^{ER/MR}, IR^{GR/MR}, and IR^{PR/MR} are >1.0 (e.g., IR^{AR/MR}>1.0 and IR^{ER/MR}>1.0 or IR^{ER/MR}>1.0 and IR^{GR/MR}>1.0). For more preferred compounds of the invention, at least three of the individual ratios selected from the group consisting of IR^{AR/MR}, IR^{ER/MR}, IR^{GR/MR}, and IR^{PR/MR} are >1.0 (e.g., IR^{AR/MR}>1.0, IR^{ER/MR}>1.0, and IR^{GR/MR}>1.0). For particularly preferred compounds of the invention, all four of the individual ratios selected from the group consisting of IR^{AR/MR}, IR^{ER/MR}, IR^{GR/MR}, and IR^{PR/MR} are >1.0 (i.e., IR^{AR/MR}>1.0, IR^{ER/MR}>1.0, IR^{GR/MR}>1.0, and IR^{PR/MR}>1.0).

For preferred compounds of the invention, at least two of the individual ratios selected from the group consisting of IR^{AR/MR}, IR^{ER/MR}, IR^{GR/MR}, and IR^{PR/MR} are ≧10.0 (e.g., IR^{AR/MR}≧10.0 and IR^{ER/MR}≧10.0 or IR^{ER/MR}≧10.0 and IR^{GR/MR}≧10.0). For more preferred compounds of the invention, at least three of the individual ratios selected from the group consisting of IR^{AR/MR}, IR^{ER/MR}, IR^{GR/MR}, and IR^{PR/MR} are ≧10.0 (e.g, IR^{AR/MR}≧10.0, IR^{ER/MR}≧10.0, and IR^{GR/MR}≧10.0). For particularly preferred compounds of the invention, all four of the individual ratios selected from the group consisting of IR^{AR/MR}, IR^{ER/MR}, IR^{GR/MR}, and IR^{PR/MR} are ≧10.0 (i.e., IR^{AR/MR}≧10.0, IR^{ER/MR}≧10.0, IR^{GR/MR}≧10.0 and IR^{PR/MR}≧10.0).

For preferred compounds of the invention, at least two of the individual ratios selected from the group consisting of IR^{AR/MR}, IR^{ER/MR}, IR^{GR/MR}, and IR^{pR/MR} are ≧100.0 (e.g, IR^{AR/MR}≧100.0 and IR^{ER/MR}≧100.0 or IR^{ER/MR}≧100.0 and IR^{GR/MR}≧100.0). For more preferred compounds of the invention, at least three of the individual ratios selected from the group consisting of IR^{AR/MR}, IR^{ER/MR}, IR^{GR/MR}, and IR^{PR/MR} are ≧100.0 (e.g, IR^{AR/MR}≧100.0, IR^{ER/MR}≧100.0, and IR^{GR/MR}≧100.0). For particularly preferred compounds of the invention, all four of the ratios selected from the group consisting of IR^{AR/MR}, IR^{ER/MR}, IR^{GR/MR}, and IR^{PR/MR} are ≧100.0 (i.e., IR^{AR/MR}≧100.0, IR^{ER/MR}≧100.0, IR^{GR/MR}≧100.0, and IR^{PR/MR}≧100.0).

The individual inhibitory ratios may be used to calculate a total inhibitory ratio (IR^{TOTAL}) according to the following equation:
IR^{TOTAL}=IR^{AR/MR}+IR^{ER/MR}+IR^{GR/MR}+IR^{AR/MR}.

Preferred compounds of the invention show an IR^{TOTAL}≧4.0. More preferred compounds of the invention show an IR^{TOTAL}≧40.0, where no single IR is lower than 1. More particularly preferred compounds of the invention show an IR^{TOTAL}≧400.0, where no single IR is lower than 10. Even more particularly preferred compounds of the invention show an IR^{TOTAL}≧1000.0. Particularly preferred compounds of the invention show an IR^{TOTAL}≧4000.0, where no single IR is lower than 10.

Uses of Compounds of Formula I

As discussed above, activation of the aldosterone (MR) receptor is associated with a variety of physiological responses, including retention of sodium and water in the kidney, increased blood pressure, stimulation of collagen production, increased plasma norepinephrine levels, and increased plasminogen activator inhibitor levels. Pathological activation of the aldosterone receptor has been implicated in a variety of disorders.

The compounds of formula I are selective antagonists of the aldosterone receptor. Accordingly, the invention further provides compositions for and methods of inhibiting aldosterone receptor activity and of treating aldosteronism.

Methods of Inhibiting Aldosterone Receptor Activity

The invention provides compositions for and methods of inhibiting aldosterone receptor activity using a compound of formula I. By “aldosterone receptor activity” is meant any function of the aldosterone receptor, including but not limited to: binding to an aldosterone receptor; translocation of the aldosterone receptor from the cytoplasm to the nucleus; receptor dimerization (including homodimerization and heterodimerization); binding of the aldosterone receptor to DNA; and transcriptional regulation of aldosterone-receptor target genes. The term “aldosterone receptor activity” also encompasses the downstream physiological consequences of aldosterone receptor signaling, including, but not limited to, retention of sodium and water in the kidney, increased blood pressure, stimulation of collagen production, increased plasma norepinephrine levels, and increased plasminogen activator inhibitor levels.

Aldosterone receptor activity may be assessed by any of the means well established in the art (see, for example, the assays discussed in the section “The novel compounds of the invention are aldosterone receptor antagonists,” above).

The method of inhibiting aldosterone receptor activity comprises contacting an aldosterone receptor with an effective amount of a compound of formula I. In one embodiment, the compound of formula I may be directly contacted to an aldosterone receptor, e.g., in vitro. In another embodiment, the compound of formula I may be contacted to a cell comprising an aldosterone receptor. Without intending to be limited by mechanism, it is thought that upon contact with the cell, the compound of formula I is taken up by the cell, resulting in direct contact of the compound with an aldosterone receptor within the cell.

As used herein, a cell that comprises an aldosterone receptor is any cell that contains an aldosterone receptor protein, including cells that endogenously express aldosterone receptor and cells that ectopically express aldosterone receptor. The target cells may be, for example, cells cultured in vitro or cells found in vivo in an organism, such as a mammal. In preferred embodiments, the cells are mammalian cells. In particularly preferred embodiments, the cells are human cells.

The aldosterone receptor expression status of a cell may be determined by any of the techniques well established in the art including Western blotting, immunoprecipitation, flow cytometry/FACS, immunohistochemistry/immunocytochemistry, Northern blotting, RT-PCR, whole mount in situ hybridization, etc. For example, monoclonal antibodies which recognize human, mouse, rabbit, rat, and chicken aldosterone receptor are commercially available from a variety of sources, e.g., from Abcam, Novus Biologicals, ALEXIS Biochemicals, and Affinity BioReagents.

Methods of Treating Aldosteronism

The invention provides compositions for and methods of treating aldosteronism using a compound of formula I.

The term “aldosteronism” refers to any disorder characterized by pathologic activation of aldosterone receptor signaling (e.g., as the result of the overproduction of aldosterone), including primary hyperaldosteronism and secondary hyperaldosteronism. As used herein, the term “primary hyperaldosteronism” refers to any disorder associated with increased secretion of the hormone aldosterone by the adrenal gland, wherein increased production of aldosterone is caused by an abnormality within the adrenal gland (e.g., a tumor). As used herein, the term “secondary hyperaldosteronism” refers to any disorder associated with increased secretion of the hormone aldosterone by the adrenal gland, wherein increased production of aldosterone is caused by an abnormality external to the adrenal gland.

Other disorders characterized by pathologic activation of aldosterone receptor signaling that may be treated in accordance with the present invention include, but are not limited to, hypertension, cardiovascular disease, renal dysfunction, liver disease, cerebrovascular disease, vascular disease, retinopathy, neuropathy (such as peripheral neuropathy), insulinopathy, edema, endothelial dysfunction, baroreceptor dysfunction, migraine headaches, hot flashes, premenstrual tension, and the like. Cardiovascular disease includes, but is not limited to, heart failure (such as congestive heart failure), arrhythmia, diastolic dysfunction (such as left ventricular diastolic dysfunction, diastolic heart failure, and impaired diastolic filling), systolic dysfunction, ischemia, hypertrophic cardiomyopathy, sudden cardiac death, myocardial and vascular fibrosis, impaired arterial compliance, myocardial necrotic lesions, vascular damage, myocardial infarction, left ventricular hypertrophy, decreased ejection fraction, cardiac lesions, vascular wall hypertrophy, endothelial thickening, fibrinoid necrosis of coronary arteries, and the like. Renal dysfunction includes, but is not limited to, glomerulosclerosis, end-stage renal disease, diabetic nephropathy, reduced renal blood flow, increased glomerular filtration fraction, proteinuria, decreased glomerular filtration rate, decreased creatinine clearance, microalbuminuria, renal arteriopathy, ischemic lesions, thrombotic lesions, global fibrinoid necrosis, focal thrombosis of glomerular capillaries, swelling and proliferation of intracapillary (endothelial and mesangial) and/or extracapillary cells (crescents), expansion of reticulated mesangial matrix with or without significant hypercellularity, malignant nephrosclerosis (such as ischemic retraction, thrombonecrosis of capillary tufts, arteriolar fibrinoid necrosis, and thrombotic microangiopathic lesions of affecting glomeruli and microvessels), and the like. Liver disease includes, but is not limited to, liver cirrhosis, liver ascites, hepatic congestion, and the like. Cerebrovascular disease includes, but is not limited to stroke. Vascular disease includes, but is not limited to, thrombotic vascular disease (such as mural fibrinoid necrosis, extravasation and fragmentation of red blood cells, and luminal and/or mural thrombosis), proliferative arteriopathy (such as swollen myointimal cells surrounded by mucinous extracellular matrix and nodular thickening), atherosclerosis, decreased vascular compliance (such as stiffness, reduced ventricular compliance and reduced vascular compliance), endothelial dysfunction, and the like. Edema includes, but is not limited to, peripheral tissue edema, hepatic congestion, splenic congestion, liver ascites, respiratory or lung congestion, and the like. Insulinopathies include, but are not limited to, insulin resistance, Type I diabetes mellitus, Type II diabetes mellitus, glucose resistance, pre-diabetic state, syndrome X, and the like.

In preferred embodiments the aldosteronism to be treated in accordance with the method of the invention is selected from the group consisting of hypertension, cardiovascular disease, renal dysfunction, edema, cerebrovascular disease, and insulinopathies. In particularly preferred embodiments the aldosteronism to be treated in accordance with the method of the invention is selected from the group consisting of hypertension, cardiovascular disease, stroke, and Type II diabetes mellitus. In particularly preferred embodiments the cardiovascular disease to be treated in accordance with the method of the invention is selected from the group consisting of heart failure, including heart failure post myocardial infarction and congestive heart failure, and left ventricular hypertrophy

The method of treating aldosteronism comprises administering to a patient in need of such treatment an effective amount of a compound of formula I. In preferred embodiments, the patient is a mammal. In particularly preferred embodiments, the patient is a human.

The term “treating” includes: (1) preventing or delaying the appearance of clinical symptoms of the state, disorder or condition developing in an animal that may be afflicted with or predisposed to the state, disorder or condition but does not yet experience or display clinical or subclinical symptoms of the state, disorder or condition; (2) inhibiting the state, disorder or condition (i.e., arresting, reducing or delaying the development of the disease, or a relapse thereof in case of maintenance treatment, of at least one clinical or subclinical symptom thereof); and/or (3) relieving the disease (i.e., causing regression of the state, disorder or condition or at least one of its clinical or subclinical symptoms). The benefit to a patient to be treated is either statistically significant or at least perceptible to the patient or to the physician.

By “treating aldosteronism” is meant any amelioration of the clinical symptoms of aldosteronism, including but not limited to, a reduction in blood pressure, reduction in edema, improved kidney function, normalized electrolyte balance in the urine, etc. Thus the methods of the invention encompass the use of a compound of formula I to prevent aldosteronism (e.g., to prevent congestive heart failure), to treat existing aldosteronism (e.g., to treat kidney dysfunction or edema), and to prevent recurrence of aldosteronism (e.g., following chemotherapy to treat an adrenal tumor, which tumor had caused primary hyperaldosteronism).

Appropriate patients to be treated according to the methods of the invention include any animal in need of such treatment. Methods for the diagnosis and clinical evaluation of aldosteronism are well established in the art. For example, aldosteronism is frequently associated with low serum level potassium, elevated plasma and/or urinary aldosterone levels, low plasma renin activity, or an abdominal CT scan showing abnormal adrenal growth, and may be diagnosed using such parameters. Thus, it is within the skill of the ordinary practitioner in the art (e.g., a medical doctor or veterinarian) to determine if a patient is in need of treatment for aldosteronism. The patient is preferably a mammal, more preferably a human, but can be any animal, including a laboratory animal in the context of a clinical trial or screening or activity experiment employing an animal model. Thus, as can be readily appreciated by one of ordinary skill in the art, the methods and compositions of the present invention are particularly suited to administration to any animal, particularly a mammal, and including, but by no means limited to, domestic animals, such as feline or canine subjects, farm animals, such as but not limited to bovine, equine, caprine, ovine, and porcine subjects, research animals, such as mice, rats, rabbits, goats, sheep, pigs, dogs, cats, etc., avian species, such as chickens, turkeys, songbirds, etc.

By “effective amount” is meant an amount of a compound of formula I sufficient to result in a therapeutic response. The therapeutic response can be any response that a user (e.g., a clinician) will recognize as an effective response to the therapy. The therapeutic response will generally be an amelioration of one or more symptoms of aldosteronism (e.g., a reduction in blood pressure, reduction in edema, improved kidney function, normalized electrolyte balance in the urine, etc.). Data obtained from cell culture assay or animal studies may be used to formulate a range of dosages for use in humans. It is further within the skill of one of ordinary skill in the art to determine an appropriate treatment duration, and any potential combination treatments, based upon an evaluation of therapeutic response. For example, a compound of formula I may be used in any of the therapeutic regimens well known in the art for aldosterone receptor antagonists.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

In one embodiment, an effective amount is from 1000 mg to 1 mg of a compound of formula I. In another embodiment, an effective amount is from 500 mg to 10 mg of a compound of formula I. In yet another embodiment, an effective amount is from 250 mg to 25 mg of a compound of formula I. For example, the aldosterone receptor antagonists may be formulated in a dosage form that contains from about 300 to about 10 mg of the active substance per unit dose, preferably from about 100 to about 25 mg of the active substance per unit dose. Depending on the disorder to be treated, a suitable therapeutically effective and safe dosage, as may readily be determined within the skill of the art, and without undue experimentation, maybe administered to subjects. For oral and parenteral administration to humans, the daily dosage level of the agent may be in single or divided doses The duration of treatment may be determined by one of ordinary skill in the art, and should reflect the nature of the disorder (e.g., a chronic versus an acute condition) and/or the rate and degree of therapeutic response to the treatment.

In the methods of treating aldosteronism, the compound of formula I may be administered in conjunction with other therapies (e.g., surgery to remove an adrenal tumor, chemotherapy to treat an adrenal tumor, or balloon angioplasty and/or stent placement of a coronary artery to treat cardiovascular disease) and/or in combination with other active agents. For example, a compound of formula I may be administered to a patient in combination with other active agents used in the treatment of hypertension and cardiovascular and renal conditions and disorders. An active agent to be administered in combination with a compound of formula I may include, for example, a drug selected from the group consisting of renin inhibitors, angiotensin II antagonists, ACE inhibitors, calcium channel blockers, diuretics having no substantial aldosterone (MR) antagonist effect, and retinoic acid. In such combination therapies the compound of formula I may be administered prior to, concurrent with, or subsequent to the other therapy and/or active agent.

Where a compound of formula I is administered in conjunction with another active agent, the individual components of such combinations may be administered either sequentially or simultaneously in separate or combined pharmaceutical formulations by any convenient route. When administration is sequential, either the compound of formula I or the second active agent may be administered first. For example, in the case of a combination therapy with another active agent, a compound of formula I and another active agent may be administered in a sequential manner in a regimen that will provide beneficial effects of the drug combination. When administration is simultaneous, the combination may be administered either in the same or different pharmaceutical compositions. For example, a compound of formula I and another active agent may be administered in a substantially simultaneous manner, such as in a single capsule or injection having a fixed ratio of these active agents or in multiple, separate capsules or injections for each agent.

When a compound of formula I is used in combination with another agent active in the methods for treating aldosteronism, the dose of each compound may differ from that when the compound is used alone. Appropriate doses will be readily appreciated by those skilled in the art.

Pharmaceutical Compositions Comprising a Compound of Formula I

While it is possible that, for use in the methods of the invention, a compound of formula I may be administered as the bulk substance, it is preferable to present the active ingredient in a pharmaceutical formulation, e.g., wherein the agent is in admixture with a pharmaceutically acceptable carrier selected with regard to the intended route of administration and standard pharmaceutical practice.

Accordingly, in one aspect, the present invention provides a pharmaceutical composition comprising at least one compound of formula I, or a pharmaceutically acceptable derivative (e.g., a salt or solvate) thereof, and, optionally, a pharmaceutically acceptable carrier. In particular, the invention provides a pharmaceutical composition comprising a therapeutically effective amount of at least one compound of formula I, or a pharmaceutically acceptable derivative thereof, and, optionally, a pharmaceutically acceptable carrier.

For the methods of the invention, a compound of formula I may be used in combination with other therapies and/or active agents. Accordingly, the present invention provides, in a further aspect, a pharmaceutical composition comprising at least one compound of formula I, or a pharmaceutically acceptable derivative thereof, a second active agent, and, optionally a pharmaceutically acceptable carrier.

When combined in the same formulation it will be appreciated that the two compounds must be stable and compatible with each other and the other components of the formulation. When formulated separately they may be provided in any convenient formulation, conveniently in such manner as are known for such compounds in the art.

The phrase “pharmaceutically acceptable” refers to molecular entities and compositions that are generally regarded as safe. In particular, pharmaceutically acceptable carriers used in the pharmaceutical compositions of this invention are physiologically tolerable and do not typically produce an allergic or similar untoward reaction (for example, gastric upset, dizziness and the like) when administered to a patient. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The compounds of the invention may be formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may be also used.

The compounds of the invention may be milled using known milling procedures such as wet milling to obtain a particle size appropriate for tablet formation and for other formulation types. Finely divided (nanoparticulate) preparations of the compounds of the invention may be prepared by processes known in the art, for example see International Patent Application No. WO 02/00196 (SmithKline Beecham).

The routes for administration (delivery) include, but are not limited to, one or more of: oral (e.g., as a tablet, capsule, or as an ingestible solution), topical, mucosal (e.g., as a nasal spray or aerosol for inhalation), nasal, parenteral (e.g., by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic including intravitreal or intracameral), transdermal, rectal, buccal, epidural and sublingual.

Therefore, the compositions of the invention include those in a form especially formulated for, e.g., parenteral, oral, buccal, rectal, topical, implant, ophthalmic, nasal or genito-urinary use. In preferred embodiments, the pharmaceutical compositions of the invention are formulated in a form that is suitable for oral delivery.

There may be different composition/formulation requirements depending on the different delivery systems. It is to be understood that not all of the compounds need to be administered by the same route. Likewise, if the composition comprises more than one active component, then those components may be administered by different routes. By way of example, the pharmaceutical composition of the present invention may be formulated to be delivered using a mini-pump or by a mucosal route, for example, as a nasal spray or aerosol for inhalation or ingestible solution, or parenterally in which the composition is formulated by an injectable form, for delivery by, for example, an intravenous, intramuscular or subcutaneous route. Alternatively, the formulation may be designed to be delivered by multiple routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile. For example, the a compound of formula I may be coated with an enteric coating layer. The enteric coating layer material may be dispersed or dissolved in either water or in a suitable organic solvent. As enteric coating layer polymers, one or more, separately or in combination, of the following can be used; e.g., solutions or dispersions of methacrylic acid copolymers, cellulose acetate phthalate, cellulose acetate butyrate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating layer polymer(s). For environmental reasons, an aqueous coating process may be preferred. In such aqueous processes methacrylic acid copolymers are most preferred.

Where appropriate, the pharmaceutical compositions can be administered by inhalation, in the form of a suppository or pessary, topically in the form of a lotion, solution, cream, ointment of dusting powder, by use of a skin patch, orally in the form of tablets containing excipients such as starch or lactose, or in capsules or ovules either alone or in admixture with excipients, or in the form of elixirs, solutions or suspensions containing flavoring or coloring agents, or they can be injected parenterally, for example intravenously, intramuscularly or subcutaneously. For buccal or sublingual administration the compositions may be administered in the form of tablets or lozenges, which can be formulated in a conventional manner.

Where the composition of the invention is to be administered parenterally, such administration includes one or more of: intravenously, intraarterially, intraperitoneally, intrathecally, intraventricularly, intraurethrally, intrasternally, intracranially, intramuscularly or subcutaneously administering the agent; and/or by using infusion techniques.

Pharmaceutical compositions of the present invention can be administered parenterally, e.g., by infusion or injection. Pharmaceutical compositions suitable for injection or infusion may be in the form of a sterile aqueous solution, a dispersion or a sterile powder that contains the active ingredient, adjusted, if necessary, for preparation of such a sterile solution or dispersion suitable for infusion or injection. This preparation may optionally be encapsulated into liposomes. In all cases, the final preparation must be sterile, liquid, and stable under production and storage conditions. To improve storage stability, such preparations may also contain a preservative to prevent the growth of microorganisms. Prevention of the action of micro-organisms can be achieved by the addition of various antibacterial and antifungal agents, e.g., paraben, chlorobutanol, or ascorbic acid. In many cases isotonic substances are recommended, e.g., sugars, buffers and sodium chloride to assure osmotic pressure similar to those of body fluids, particularly blood. Prolonged absorption of such injectable mixtures can be achieved by introduction of absorption-delaying agents, such as aluminium monostearate or gelatin.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

For parenteral administration, the compound is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Sterile injectable solutions can be prepared by mixing a compound of formula I with an appropriate solvent and one or more of the aforementioned carriers, followed by sterile filtering. In the case of sterile powders suitable for use in the preparation of sterile injectable solutions, preferable preparation methods include drying in vacuum and lyophilization, which provide powdery mixtures of the aldosterone receptor antagonists and desired excipients for subsequent preparation of sterile solutions.

The compounds according to the invention may be formulated for use in human or veterinary medicine by injection (e.g., by intravenous bolus injection or infusion or via intramuscular, subcutaneous or intrathecal routes) and may be presented in unit dose form, in ampoules, or other unit-dose containers, or in multi-dose containers, if necessary with an added preservative. The compositions for injection may be in the form of suspensions, solutions, or emulsions, in oily or aqueous vehicles, and may contain formulatory agents such as suspending, stabilizing, solubilizing and/or dispersing agents. Alternatively the active ingredient may be in sterile powder form for reconstitution with a suitable vehicle, e.g., sterile, pyrogen-free water, before use.

The compounds of the invention can be administered (e.g., orally or topically) in the form of tablets, capsules, ovules, elixirs, solutions or suspensions, which may contain flavoring or coloring agents, for immediate- , delayed- , modified- , sustained- , pulsed- or controlled-release applications.

The compounds of the invention may also be presented for human or veterinary use in a form suitable for oral or buccal administration, for example in the form of solutions, gels, syrups, mouth washes or suspensions, or a dry powder for constitution with water or other suitable vehicle before use, optionally with flavoring and coloring agents. Solid compositions such as tablets, capsules, lozenges, pastilles, pills, boluses, powder, pastes, granules, bullets or premix preparations may also be used. Solid and liquid compositions for oral use may be prepared according to methods well-known in the art. Such compositions may also contain one or more pharmaceutically acceptable carriers and excipients which may be in solid or liquid form.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

The compositions may be administered orally, in the form of rapid or controlled release tablets, microparticles, mini tablets, capsules, sachets, and oral solutions or suspensions, or powders for the preparation thereof. In addition to the new solid-state forms of pantoprazole of the present invention as the active substance, oral preparations may optionally include various standard pharmaceutical carriers and excipients, such as binders, fillers, buffers, lubricants, glidants, dyes, disintegrants, odorants, sweeteners, surfactants, mold release agents, antiadhesive agents and coatings. Some excipients may have multiple roles in the compositions, e.g., act as both binders and disintegrants.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate and colloidal silicon dioxide.

Examples of suitable pharmaceutically acceptable odorants for the oral compositions include but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Examples of useful pharmaceutically acceptable coatings for the oral compositions, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable sweeteners for the oral compositions include but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulfate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

The compounds of the invention may also, for example, be formulated as suppositories e.g., containing conventional suppository bases for use in human or veterinary medicine or as pessaries e.g., containing conventional pessary bases.

The compounds according to the invention may be formulated for topical administration, for use in human and veterinary medicine, in the form of ointments, creams, gels, hydrogels, lotions, solutions, shampoos, powders (including spray or dusting powders), pessaries, tampons, sprays, dips, aerosols, drops (e.g., eye ear or nose drops) or pour-ons.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax, sorbitan monostearate, a polyethylene glycol, liquid paraffin, polysorbate 60, cetyl esters wax, cetearyl alcohol, 2-octyldodecanol, benzyl alcohol, and water. Such compositions may also contain other pharmaceutically acceptable excipients, such as polymers, oils, liquid carriers, surfactants, buffers, preservatives, stabilizers, antioxidants, moisturizers, emollients, colorants, and odorants.

Examples of pharmaceutically acceptable polymers suitable for such topical compositions include, but are not limited to, acrylic polymers; cellulose derivatives, such as carboxymethylcellulose sodium, methylcellulose or hydroxypropylcellulose; natural polymers, such as alginates, tragacanth, pectin, xanthan and cytosan.

Examples of suitable pharmaceutically acceptable oils which are so useful include but are not limited to, mineral oils, silicone oils, fatty acids, alcohols, and glycols.

Examples of suitable pharmaceutically acceptable liquid carriers include, but are not limited to, water, alcohols or glycols such as ethanol, isopropanol, propylene glycol, hexylene glycol, glycerol and polyethylene glycol, or mixtures thereof in which the pseudopolymorph is dissolved or dispersed, optionally with the addition of non-toxic anionic, cationic or non-ionic surfactants, and inorganic or organic buffers.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetriacetic acid (EDTA), thiourea, tocopherol and butyl hydroxyanisole.

Suitable examples of pharmaceutically acceptable moisturizers include, but are not limited to, glycerine, sorbitol, urea and polyethylene glycol.

Suitable examples of pharmaceutically acceptable emollients include, but are not limited to, mineral oils, isopropyl myristate, and isopropyl palmitate.

The compounds may also be dermally or transdermally administered, for example, by use of a skin patch.

For ophthalmic use, the compounds can be formulated as micronized suspensions in isotonic, pH adjusted, sterile saline, or, preferably, as solutions in isotonic, pH adjusted, sterile saline, optionally in combination with a preservative such as a benzylalkonium chloride.

As indicated, the compound of the present invention can be administered intranasally or by inhalation and is conveniently delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoroethane, a hydrofluoroalkane such as 1,1,1,2-tetrafluoroethane (HFA 134AT) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant, e.g., sorbitan trioleate.

Capsules and cartridges (made, for example, from gelatin) for use in an inhaler or insufflator may be formulated to contain a powder mix of the compound and a suitable powder base such as lactose or starch.

For topical administration by inhalation the compounds according to the invention may be delivered for use in human or veterinary medicine via a nebulizer.

The pharmaceutical compositions of the invention may contain from 0.01 to 99% weight per volume of the active material. For topical administration, for example, the composition will generally contain from 0.01-10%, more preferably 0.01-1% of the active material.

### EXAMPLES

The features and advantages of the invention are more fully shown by the following non-limiting examples.

General Methods

Commercially available reagents and solvents (HPLC grade) were used without further purification. Polystyrene-carbonate resin was obtained commercially from Argonaut.

Microwave irradiation was carried out using a CEM Discover focused microwave reactor.

Solvents were removed using a GeneVac Series I without heating or a Genevac Series II with VacRamp at 40° C.

Purification of compounds by flash chromatography column was performed using silica gel, particle size 40-63 μm (230-400 mesh) obtained from Silicycle. Purification of compounds by preparative HPLC was performed on Gilson systems using reverse phase ThermoHypersil-Keystone Hyperprep HS C18 columns (12 μm, 100×21.2 mm), gradient 20-100% B (A=water/0.1% TFA, B=acetonitrile/0.1% TFA) over 9.5 min, flow=30 mL/min, injection solvent 2:1 DMSO:acetonitrile (1.6 mL), UV detection at 215 nm.

¹H NMR and ¹⁹F spectra were recorded on a Bruker 400 MHz AV spectrometer in deuterated solvents. Chemical shifts (6) are in parts per million and coupling constants are expressed in Hz. Thin-layer chromatography (TLC) analysis was performed with Kieselgel 60 F₂₅₄ (Merck) plates and visualized using UV light.

Analytical HPLC-MS was performed on Agilent HP 1100, Waters 600 or Waters 1525 LC systems using reverse phase Hypersil BDS C18 columns (5 μm, 2.1×50 mm), gradient 0-95% B (A=water/0.1% TFA, B=acetonitrile/0.1% TFA) over 2.10 min, flow=1.0 mL/min. UV spectra were recorded at 215 nm using a Gilson G1315A Diode Array Detector, G1214A single wavelength UV detector, Waters 2487 dual wavelength UV detector, Waters 2488 dual wavelength UV detector, or Waters 2996 diode array UV detector. Mass spectra were obtained over the range m/z 150 to 850 at a sampling rate of 2 scans per second or 1 scan per 1.2 seconds using Micromass LCT with Z-spray interface or Micromass LCT with Z-spray or MUX interface. Data were integrated and reported using OpenLynx and OpenLynx Browser software.

### Example 1

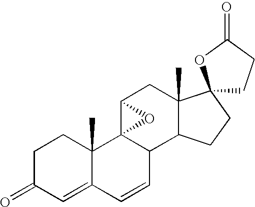

The dieneone intermediate of Example 1 was prepared from the corresponding eneone as described in Helv. Chim. Acta. Vol 80, 1997, 566. To a solution of the 6,7-saturated steroid (3.12 g, 8.76 mmol) in dioxane (50 mL), DDQ (2.18 g, 9.6 mmol) and 5.0 N HCl in dioxane (135 mL) were added. The mixture was stirred at 20° C. for 90 min. The dark-coloured solution was diluted with methylene chloride and filtered through neutral alumina. Evaporation and crystallization of the crude product from CH₂Cl₂/MeOH/Et₂O furnished the title compound.

### Example 2

### Pregna-4,6,9(11)-triene-17β-ol-3-one-21-carboxylate γ lactone (Trienone A)

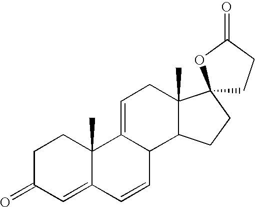

The trieneone intermediate of Example 2 can be prepared, for example, by decarboxylation of the compound of Example 5 with treatment of heat, water and an alkali halide followed by oxidation with DDQ to install the 6,7 alkene. Preparation of this compound has been described in WO 97/21720 (pages 84-85, 89), WO 98/25948 and WO 04/085458

### Example 3

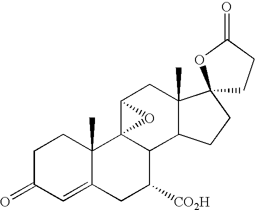

The intermediate of Example 3 was prepared as described in WO 98/25948 (Example 47J, page 307)

### Example 4

### 3-Methoxy-3,5,9(11)-triene-17 oxirane intermediate (Oxirane B)

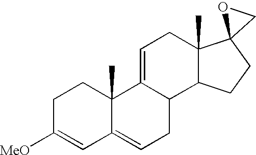

The intermediate of Example 4 was prepared by conversion of the corresponding 17-keto-compound to the epoxide by treatment with soulfonium ylide as described in WO 97/21720 (pages 84 and 89)

### Example 5

### 3-Methoxy-3,5,9(11)-triene-17 spirolactone-22-methoxycarbonyl intermediate (Spirolactone Ester C)

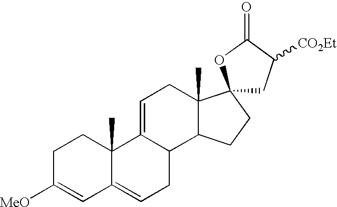

The intermediate of Example 5 was prepared from the epoxide of Example 4 by treatment with CH₂(COOEt)₂ and base as described in WO 97/21720 (pages 84 and 89).

### Example 6

### C-3, C-21 Dione C-7 Aldehyde Intermediates (Aldehydes D and E)

### Intermediate 6-1

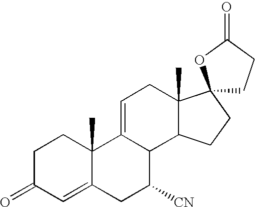

To the intermediate of Example 2 (trienone A; 3.23 g, 9.54 mmol) was added dry THF (30 mL) under nitrogen. Diethylaluminium cyanide (1 M in PhMe, 2.5 eq, 24 mL) was added and the mixture heated at reflux for 30 min. The mixture was allowed to cool and poured onto ice (100 g). Aqueous sodium hydroxide (1 M, 30 mL) was added, followed by saturated sodium potassium tartrate (75 mL). The mixture was extracted with dichloromethane (2×50 mL), the organic layers combined and evaporated. The residue was purified by flash chromatography eluting with heptane-ethyl acetate (1:2) to yield the required nitrile (2.37 g, 68%) as a 3:1 (alpha/beta ratio) mixture of diastereomers. The alpha nitrile was obtained following prep HPLC of 500 mg of 3:1 (alpha/beta ratio) nitrile. Yield: 287 mg (57%, 7 alpha from 500 mg of 3:1 mixture of α,β-C-7 nitrile). Mass spectrum (ES-MS (+ve)) 366 [M+H]⁺, Retention time 1.82 min, 100% UV. ¹H-NMR (CDCl₃, 400 MHz): δ 5.94 (1H, s, C₄—H), 5.83 (1H, dd, C₁₁—H), 3.19 (1H, m), 2.86 (1H, ddd), 2.72-2.19 (11H, m), 2.06-1.88 (4H, m), 1.80 (1H, m), 1.51 (1H, m), 1.39 (3H, s, Me), 0.98 (3H, m). Note: The C-7 nitrile 3:1 (alpha/beta ratio) mixture of diastereomers was used to prepare Aldehyde D-3: 1 C-7-alpha/beta ratio

### Intermediate 6-2

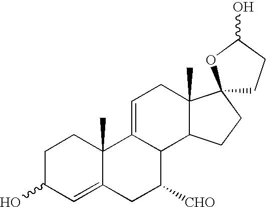

Intermediate 6-1 was dissolved in anhydrous 1,2-dimethoxyethane (15 mL) under nitrogen and cooled to 0° C. Diisobutylaluminium hydride (1.5 M in toluene, 6 eq, 3.1 mL) was added dropwise and the mixture stirred at 0-5° C. for 2 hrs. The reaction mixture was quenched with 3 N hydrochloric acid (3 mL), diluted with water (20 mL), and extracted with dichloromethane (2×10 mL). The organic extracts were combined, dried over anhydrous sodium sulfate and evaporated to give the crude diol aldehyde as an isomeric mixture of diastereomers at C-3 and C-21 which was used without further purification. Yield: 196 mg. Mass spectrum (ES-MS (+ve)) 355 [M+H−H₂O]⁺, Retention time 1.07 min.
Aldehyde D (3: 1 C-7 alpha/beta ratio)

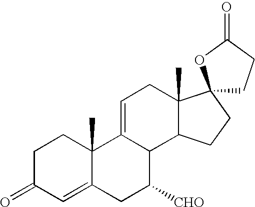

Intermediate 6-2 (195 mg, 523 μmol) was dissolved in dry dichloromethane (3 mL) containing activated 4A molecular sieves (250 mg) under nitrogen. Tetrapropyl ammonium perruthenate (5 mol %, 9.2 mg, 26 μmol) and N-methylmorpholine N-oxide (134 mg, 1.15 mmol) were added and the mixture stirred at room temperature for 2 hrs. After this time the reaction mixture was evaporated to dryness, re-dissolved in DCM (1.5 mL) and filtered through a (ca 2 mm) pad of silica. Evaporation of the filtrate gave the crude aldehyde as a green-black gum which was used without further purification. ¹H-NMR (CDCl₃, 400 MHz): δ 9.73, 9.49 (1H, 2×d, CHO)

Aldehyde E (C-7 alpha aldehyde)

Diastereopure aldehyde E (C-7 alpha aldehyde) was prepared using the above methods for aldehyde D except that diasteropure C-7 alpha nitrile was used in place of the diasteromeric mixture of alpha/beta C-7 nitriles (3:1 alpha/beta ratio). Yield: 87 mg. Mass spectrum (ES-MS (+ve)) 369 [M+H]⁺, Retention time 1.22 min. ¹H-NMR (CDCl₃, 400 MHz): 9.49 (1H, d, CHO).

### Example 7

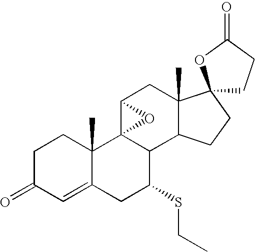

The compound of Example 1 (1.75 g, 4.9 mmol) was dissolved in ethanethiol (7.42 mL, 141 mmol) under nitrogen. The reaction mixture was heated to 30° C. Sodium metal (113 mg, 4.9 mmol) was added in one portion and further ethanethiol (2 mL) added. The reaction was re-flushed with nitrogen and heated to 40° C. for 4 h, allowed to cool and stirred at ambient temperature for 15 h. Saturated aqueous ammonium chloride (˜90 mL) was added and the aqueous phase extracted with dichloromethane. The organic layers were washed with saturated brine solution, dried over MgSO₄ (magnesium sulfate) and the solvent evaporated in vacuo. The residue was purified by flash column chromatography using 1:2 ethyl acetate:heptane as eluent to give the required product. Yield: 857 mg (41%). Mass spectrum (ES-MS (+ve)) 416 [M+H]⁺, retention time: 1.29 min. ¹H-NMR (CDCl₃, 400 MHz): δ 5.9 (1H, s, C₄—H), 3.2 (1H, s, CH), 3.1 (1H, d, CH), 2.9 (1H, d, CH), 2.7(1H, d, CH), 2.65-1.70 (19H, m, CH), 1.5(3H, s, CH₃), 1.4(2H, d, CH), 1.25 (3H, t, CH₃), 1.0 (3H, s, CH₃).

### Example 8

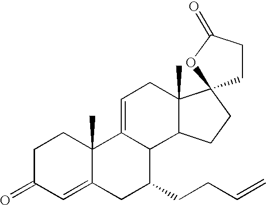

Copper I chloride, 4.7 mg, 0.047 mmol) and LiCl (2.99 mg, 0.07 mmol) were mixed in THF (1.5 mL) and diethyl ether (1.5 mL) under nitrogen. Trienone A (Example 2) (60 mg, 0.177 mmol) in THF (1.0 mL) was added. The mixture was cooled to −30° C. and a 0.5M solution of 3-butenyl magnesium bromide (0.5 mL, 0.251 mmol) in THF (tetrahydrofuran) added dropwise over 10 min. The reaction mixture was stirred at −30° C. for 1.5 h, 5M HCl (hydrochloric acid, 0.5 mL) added and the reaction stirred at −30° C. for 5 min. 2N NH₃ (ammonia, 1.3 mL) was added during warming of the reaction to room temperature. The mixture was extracted with diethyl ether, washed with 0.5M aqueous NH₃ and water, dried over MgSO₄ and the solvent removed in vacuo. The residue (85% α isomer and 15% β isomer) was purified by column chromatography eluting with EtOAc (ethyl acetate):heptane (2:3). The material obtained was re-purified by column chromatography and pure alpha isomer was obtained (7 mg). Yield: 25 mg (40% for 85% α isomer and 15% βisomer mixture). Mass spectrum (ES-MS (+ve) 395 [M+H]⁺, Retention time 1.59 min. ¹H-NMR (pure 7-alpha; CDCl₃, 400 MHz):δ 5.80 (1H, s, C₄—H), 5.65 (1H, m, olefin CH), 5.55 (1H, d, C₁₁—H), 4.95 (2H, m, olefin CH₂), 2.60-1.40 (21H, m, alkyl —H), 1.35 (3H, s, Me), 1.25 (2H, m, alkyl —H), 0.90 (3H, s, Me).

### Example 9

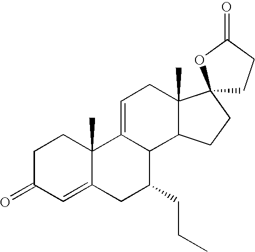

Copper I chloride (16.5 mg, 0.17 mmol) and lithium chloride (10.5 mg, 0.25 mmol) were mixed in diethyl ether (4.5 mL) and THF (4.5 mL) under an inert atmosphere and stirred for 0.5 h until the lithium chloride had dissolved. Trienone A (Example 2) (210 mg, 0.62 mmol) in tetrahydrofuran (6 mL) was added and the mixture cooled to −30° C. n-Propylmagnesium chloride in diethylether (2 M, 0.45 mL) was added dropwise over 15 min. The reaction mixture was allowed to warm to −10° C. overnight. Hydrochloric acid (5 N, 1.2 mL) was added and the reaction stirred to room temperature. Aqueous ammonia (2 N, 3 mL) was added and the reaction mixture extracted with diethyl ether, washed with aqueous ammonia (0.5 N) and water. The organic phase was dried over MgSO₄ and the solvent evaporated in vacuo to give a solid which was purified by silica chromatography using 2:1 heptane:ethyl acetate to give the required product. Yield: 23 mg (15%). Mass spectrum (ES-MS (+ve)) 383 [M+H]⁺, retention time 1.60 min. ¹H-NMR (CDCl₃, 400 MHz):δ5.8 (1H, s, C₄—H), 5.6 (1H, d, C₁₁—H), 2.65-1.1 (25H, m, CH), 0.95 (3H, s, CH₃), 0.85 (3H, t, CH₃), 0.8 (1H, m, CH).

### Example 10

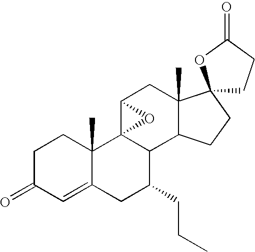

The compound of Example 10 (23 mg, 0.06 mmol) was dissolved in dichloromethane (2 mL). m-CPBA (3-Chloroperoxybenzoic acid, 16.9 mg, 0.10 mmol) was added and the mixture stirred at room temperature for 2 h. Further 3-chloroperoxybenzoic acid (16.9 mg, 0.10 mmol) was added and the reaction stirred for 1 h. The reaction was diluted with dichloromethane and washed with 10% aqueous potassium iodide (3×3 mL), saturated aqueous sodium thiosulphate (2×3 mL), saturated aqueous sodium hydrogen carbonate (3 mL), dried over MgSO₄ and the solvent evaporated in vacuo. Purification by flash chromatography with 2:3 ethyl acetate:cyclohexane gave the required product. Yield: 12 mg (50%). Mass spectrum (ES-MS (+ve)) 399 [M+H]⁺, retention time 1.48 min. ¹H-NMR (CDCl₃, 400 MHz):δ5.85 (1H, s, C₄—H), 3.0 (1H, d, CH), 2.65-1.1 (26H, m, CH), 1.0 (3H, s, CH₃), 0.9 (3H, t, CH₃)

### Example 11

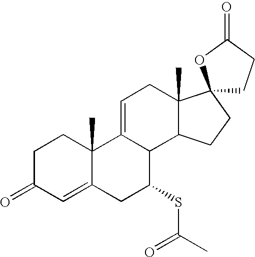

The compound of Example 2 (Trienone A) (1.25 g, 3.7 mmol) was dissolved in methanol (15 mL) under nitrogen and thioacetic acid (2 mL, 7.8 mmol) added. The reaction mixture was refluxed for 20 h. The solvent was removed under reduced pressure and the crude residue partitioned between dichloromethane and saturated aqueous sodium hydrogen carbonate. The organic phase was dried over MgSO₄ and the solvent evaporated in vacuo. The residue was purified by flash chromatography using 2:1 cyclohexane:ethyl acetate as eluent. Further purification by prep HPLC gave the required product. Yield: 490 mg (32%). Mass spectrum (ES-MS (+ve)) 415 [M+H]⁺, retention time 1.44 min. ¹H-NMR (CDCl₃, 400 MHz): δ5.75 (1H, s, C₄—H), 5.65 (1H, d, C₁₁—H), 4.1 (1H, d, CH), 3.0 (1H, d, CH), 2.7-1.45 (20H, m, CH), 1.4 (3H, s, CH₃), 0.95 (3H, s, CH₃)

### Example 12

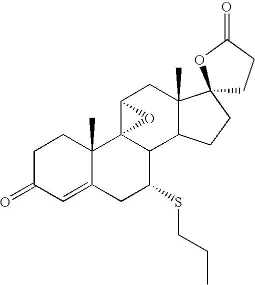

The compound of Example 1 (120 mg, 0.34 mmol) was suspended in n-propylthiol (0.62 mL, 6.8 mmol) under nitrogen. The reaction mixture was heated to 30° C. Tetrahydrofuran (2 mL) was added followed by sodium metal (7.8 mg, 0.34 mmol). The reaction was re-flushed with nitrogen, heated to 40° C. for 1 h and allowed to cool to ambient temperature. Saturated aqueous ammonium chloride (˜5 mL) was added and the aqueous phase extracted with dichloromethane. The organics were washed with saturated brine solution, dried over MgSO₄ and the solvent evaporated in vacuo to give a yellow solid which was purified by prep-HPLC to give the required product. Yield: 68 mg (46%). Mass spectrum (ES-MS (+ve)) 431 [M+H]⁺, retention time 1.38 min. ¹H-NMR (CDCl₃, 400 MHz):δ 5.9 (1H, s, C₄—H), 3.2 (1H, s, CH), 3.05 (1H, d, CH), 2.9 (1H, d, CH), 2.7-1.55 (19H, m, CH), 1.5 (3H, s, CH₃), 1.4 (2H, m, CH), 1.05 (6H, m, 2×CH₃).

### Example 13

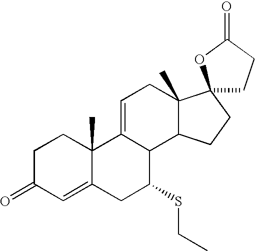

The compound of Example 2 (Trienone A; 2.6 g, 7.7 mmol), was dissolved in ethanethiol (11.5 mL, 15.4 mmol) under nitrogen. The reaction mixture was heated to 30° C. Sodium metal (177 mg, 7.7 mmol) was added in one portion and the reaction was re-flushed with nitrogen. The reaction mixture was heated to 60° C. for 2 h, cooled and stirred at ambient temperature for 15 h. Saturated aqueous ammonium chloride (˜90 mL) was added and the aqueous phase extracted with dichloromethane. The organic layer was washed with saturated brine solution, dried over MgSO₄ and the solvent evaporated in vacuo. The residue was purified by flash chromatography using 1:2 ethyl acetate:heptane as eluent. Further purification by prep-HPLC gave the required product. Yield: 480 mg (15%). Mass spectrum (ES-MS (+ve)) 401 [M+H]⁺, retention time 1.49 min. ¹H-NMR (CDCl₃, 400 MHz):δ5.8 (1H, s, C₄—H), 5.65 (1H, d, C₁₁—H), 3.2 (1H, m, CH), 2.9 (1H, d, CH) 2.7-1.8 (18H, m, CH), 1.5 (1H, m, CH), 1.4 (3H, s, CH₃), 1.2 (3H, t, CH₃), 0.95 (3H, s, CH₃)

### Example 14

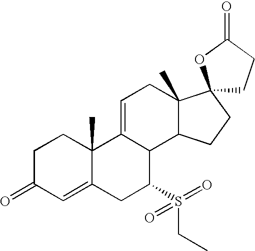

The compound of Example 13 (160 mg, 0.28 mmol 70% pure) was dissolved in dichloromethane (10 mL). 3-Chloroperoxybenzoic acid (247 mg, 1.43 mmol, 75% purity) was added and stirred at ambient temperature for 1.5 h. The reaction mixture was diluted with dichloromethane (10 mL) and washed with 10% aqueous potassium iodide (3×15 mL), saturated aqueous sodium thiosulphate (2×15 mL), saturated aqueous sodium hydrogen carbonate (15 mL), dried over MgSO₄ and the solvent evaporated in vacuo. Purification by prep HPLC followed by flash chromatography using 2:1 heptane:ethyl acetate as eluent gave the required product. Yield: 30 mg (25%). Mass spectrum (ES-MS (+ve)) 433 [M+H]⁺, retention time 1.13 min. ¹H-NMR (CDCl₃, 400 MHz):δ 5.9 (1H, s, C₄—H), 5.75 (1H, d, C₁₁—H), 3.4 (1H, m, CH), 3.0-1.6 (20H, m, CH), 1.45

H, s, CH₃), 1.35 (3H, t, CH₃), 0.95 (3H, s, CH₃).

### Example 15

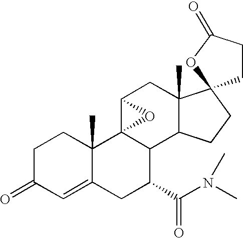

The compound of Example 3 (100 mg, 0.025 mmol) was dissolved in DCM (dichloromethane, 10 mL) and cooled 0° C., 1-chloro-N,N-2-trimethyl-1-propenylamine (0.036 mL, 0.275 mmol) was added dropwise before stirring for 1 h whilst reaching room temperature. In another flask dimethyl amine hydrochloride (0.022 g, 0.27 mmol) and triethylamine (0.076 mL, 0.054 mmol) were diluted with DCM (5 mL) and the mixture added dropwise to the acid chloride at 0° C. The reaction was allowed to warm room temperature and stirred at room temperature for 16 h. The reaction mixture was diluted with DCM and the organic phase washed with 0.5M HCl, water, dried over MgSO₄ and the solvent removed in vacuo. The residue was purified by flash chromatography eluting with DCM:Methanol (97:3). Yield: 90 mg (84%). Mass spectrum (ES-MS (+ve) 428 [M+H]⁺, Retention time 1.00 min. ¹H-NMR (CDCl₃, 400 MHz): δ 5.75 (1H, s, C₄—H), 3.30 (1H, m, NCOCH), 3.15 and 2.95 (2×3H, 2×S, 2×NMe), 3.00 (1H, d, C₁₁—H), 2.75 (1H, m, alkyl-H), 2.60-1.50 (16H, m, alkyl —H), 1.40 (3H, s, Me), 1.35 (1H, m, alkyl —H), 1.0 (3H, s, Me).

### Example 17

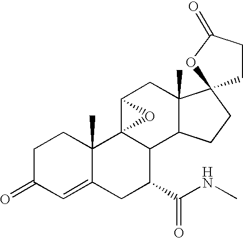

The compound of Example 17 was prepared as described in Example 15 using methylamine in place of dimethyl amine hydrochloride. Yield: 52 mg (52%). Mass spectrum (ES-MS (+ve) 414 [M+H]⁺, Retention time 1.00 min. ¹H-NMR (CDCl₃, 400 MHz): δ 6.20 (1H, br, NH), 5.95 (1H, s, C₄—H), 3.20 (1H, d, C₁₁—H), 2.95 (2H, m, alkyl-H), 2.75 (3H, S, NMe), 2.65-1.60 (16H, m, alkyl —H), 1.40 (3H, s, Me), 1.35 (1H, m, alkyl —H), 1.0 (3H, s, Me).

### Example 18

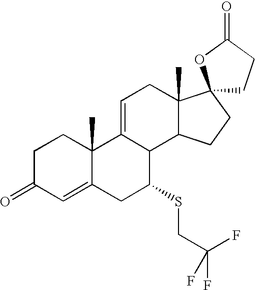

The compound of Example 2 (Trienone A; 200 mg, 0.6 mmol) was dissolved in 2,2,2-trifluoro ethanethiol (2 mL) under nitrogen. The reaction mixture was heated to 30° C. Sodium metal (14 mg, 0.6 mmol) was added in one portion and the reaction was re-flushed with nitrogen. The reaction mixture was heated to 60° C. for 2 h, allowed to cool and stirred at ambient temperature for 15 h. Saturated aqueous ammonium chloride was added to pH 7 and the aqueous phase extracted with dichloromethane. The organic layers were washed with saturated brine solution, dried over magnesium sulphate and the solvent evaporated in vacuo. The residue was purified by flash chromatography using 1:2 ethyl acetate:heptane. Further purification by prep-HPLC gave the required product. Yield: 107 mg (39%). Mass spectrum (ES-MS (+ve)) 455 [M+H]⁺, retention time 1.49 min. ¹H-NMR (CDCl₃, 400 MHz):δ 5.8 (1H, s, C₄—H), 5.7 (1H, d, C₁₁—H), 3.4 (1H, m, CH), 3.05-1.5 (20H, m, CH), 1.4 (3H, s, CH₃), 0.95 (3H, s, CH₃)

### Example 19

### Intermediate 19-1

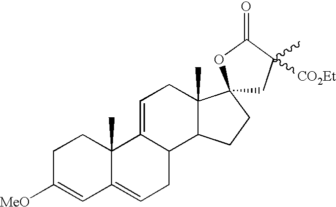

To sodium ethoxide (201 mg, 2.83 mmol) was added ethanol (6 mL). The compound of Example 5 (Spirolactone Ester C; 1.19 g, 2.8 mmol) was added followed by methyl iodide (173 μl, 2.8 mmol). The reaction mixture was warmed to 45° C. and stirred for 1 h. The reaction mixture was reduced to half the original solvent volume by evaporation in vacuo and quenched with glacial acetic acid (25 mg, 420 μmol). The product was precipitated by the slow addition of water (6 mL over 20 min). The precipitate was filtered and dried in vacuo to give the title compound as a yellow powder. Yield: 1.10 g (90%). Mass spectrum (ES-MS (+ve)) 441 [M+H]⁺, Retention time 1.91 min, 92% UV. ¹H-NMR (d₆-DMSO, 400 MHz): δ 5.51 (1H, dd, C₁₁—H), 5.23 (1H, dd, C₆—H), 5.20 (1H, s, C₄—H), 4.18 (2H, 2×q, CH₂Me), 3.51 (3H, s, OMe), 2.89 (1H, ddd, C₈—H), 2.51-1.67 (13H, m), 1.49, 1.41 (3H, 2×s, C₂₂—Me), 1.43 (1H, m), 1.40-1.29 (1H, m), 1.21 (3H, 2×t, Me), 1.09 (3H, s, Me), 0.88 (3H, s, Me).

### Example 19

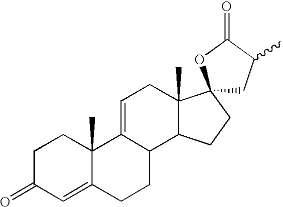

Intermediate 19-1 (430 mg, 976 μmol) was dissolved in DMF (N,N-dimethylformamide, 3 mL). Sodium chloride (114 mg, 2 mmol) and water (50 μl) was added. The mixture was heated at reflux under nitrogen for 72 h. After this time a mixture of starting material and decarboxylated product was observed in addition to the corresponding deprotected enone's, the major constituent being the title compound. The reaction mixture was diluted with ethyl acetate (15 mL) and extracted with water (4×10 mL). Evaporation gave a yellow gum which was treated with ethanol-water-acetic acid 1:1:1 (6 mL) at reflux for 0.5 h. Evaporation gave a residue which was purified by HPLC to give the title compound as a clear film. Yield: 165 mg (45%). Mass spectrum (ES-MS (+ve)) 355 [M+H]⁺, Retention time 1.54 min, 100% UV. ¹H-NMR (CDCl₃, 400 MHz): [symbol] 5.75 (1H, s, C₄—H), 5.55 (1H, dd, C₁₁—H), 2.86 (1H, m, C₈—H), 2.75-1.35 (17H, m), 1.36 (3H, 2×s, Me) 1.28, 1.26 (3H, 2×d, C₂₂-Me), 1.11 (1H, m), 0.99, 0.99 (3H, s, Me).

### Example 20

### Intermediate 20-1

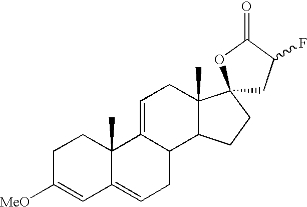

Intermediate 21-1 (160 mg, 360 μmol) was dissolved in DMF (2 mL). Sodium chloride (44 mg, 740 μmol) and water (100 μl) were added and the mixture heated at reflux for 24 h. After this time the mixture was cooled and evaporated. The residue was loaded onto silica (DCM) and purified by chromatography on silica eluting with heptane-ethyl acetate (9:1 to 7:4). The intermediate enol-ether was obtained as a pale yellow gum. Yield: 88 mg (66%). Mass spectrum (ES-MS (+ve)) 373 [M+H]⁺, Retention time 1.85 min. ¹H-NMR (d₆-DMSO, 400 MHz):δ 5.73, 5.59, 5.46, 5.33 (1H, 2×dt, C₂₂—H—F), 5.41 (1H, dd, C₁—H), 5.19 (1H, dd, C₆—H), 5.12 (1H, s, C₄—H), 3.44 (3H, s, OMe), 2.92 (1H, m), 2.52-1.03 (15H, m), 1.14 (3H, 2×s, Me), 0.84, 0.72 (3H, 2×s, Me). ¹⁹F-NMR (d₆-DMSO, 376 MHz): −190.93, −196.02.

### Example 20

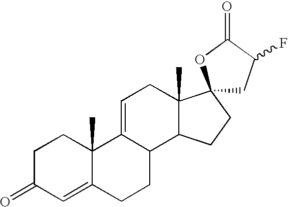

Intermediate 19-1 (88 mg, 236 μmol) was hydrolysed at 100° C. in water-ethanol-acetic acid 1:1:1 (5 mL) for 30 min. After evaporation, the residue was chromatographed on silica eluting with heptane-ethyl acetate (3:1) to give the title compound as a colourless solid (3:1 mixture of diastereomers). Yield: 31 mg (36%). Mass spectrum (ES-MS (+ve)) 359 [M+H]⁺, Retention time 1.50 min, 98% UV. ¹H-NMR (CDCl₃, 400 MHz): δ 5.76 (1H, s, C₄—H), 5.63 (1H, dd, C₁₁—H), 5.43-5.12 (1H, 2×dt, C₂₂—H—F), 2.79 (1H, m), 2.61-1.47 (15H, m), 1.36 (3H, s), 1.35-1.04 (2H, m), 0.99 (3H, s).

### Example 21

### Intermediate 21-1

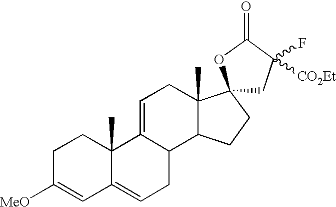

The compound of Example 5 (Spirolactone Ester C, 1.06 g, 2.49 mmol) was dissolved in dry THF (10 mL) under nitrogen. NaH (sodium hydride, 60% in mineral oil, 104 mg, 2.61 mmol) was added and the mixture stirred at room temperature for 5 min. Powdered Selectfluor™ (924 mg, 2.61 mmol) was added and the mixture stirred for 1 h. The reaction was diluted with t-butylmethyl ether (50 mL) and extracted with 5% aqueous sulfuric acid (2×25 mL), water (50 mL), saturated brine (50 mL) and dried over anhydrous sodium sulfate. Evaporation gave the required fluoro-lactone as a pale yellow solid (˜2:1 mixture of diasteromers). Yield: 980 mg (89%). Mass spectrum (ES-MS (+ve)) 445 [M+H]⁺, Retention time 1.96 min, 85% UV. ¹H-NMR (d₆-DMSO, 400 MHz): δ 5.52 (1H, dd, C₁₁—H), 5.23(1H, dd, C₆—H), 5.20(1H, s, C₄—H), 4.29 (2H, 2×q, CH₂Me), 3.53 (3H, s, OMe), 3.10-1.32 (16H, m), 1.27 (3H, 2×t, Me), 1.13, 1.08 (3H, 2×s, Me), 0.98, 0.89 (3H, 2×s, Me). ¹⁹F-NMR (d₆-DMSO, 376 MHz): −156.10, −156.58.

### Example 21

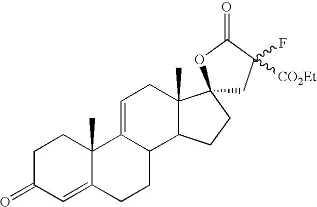

Intermediate 21-1 (150 mg, 340 μmol) was dissolved in ethanol-water-acetic acid 1:1:1 (1.5 mL) and heated at reflux for 1 h. The reaction mixture was diluted with water (5 mL) and extracted with DCM (2×5 mL). The organic layers were combined and dried over anhydrous sodium sulfate. Evaporation gave 118 mg crude product which was purified by chromatography in silica eluting with ethyl acetate-heptane 1:1 to give the title compound as a colourless gum (3:1 mixture of diastereomers). Yield: 57 mg (40%). Mass spectrum (ES-MS (+ve)) 431 [M+H]⁺, Retention time 1.63 min, 91% UV. ¹H-NMR (CDCl₃, 400 MHz): δ 5.75 (1H, s, C₄—H), 5.57 (1H, dd, C₁₁—H), 4.35 (2H, 2×q, CH₂Me), 3.00-1.85 (15H, m), 1.58 (1H, m), 1.40-1.21 (4H, m), 1.08 (1H, m), 1.04+1.00 (3H, 2×s, Me), 0.90 (3H, 2×t, Me). ¹⁹F-NMR (CDCl₃, 376 MHz): −156.13, −157.46.

### Example 22

### Intermediate 22-1

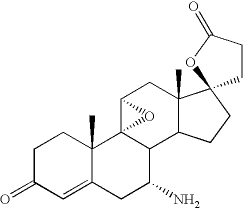

The compound of Example 3 (1.0 g, 2.5 mmol) and triethylamine (0.4 mL, 2.87 mmol) were dissolved in THF (10 mL), cooled to 0° C. Isobutyl chloroformate (0.37 mL, 2.87 mmol) in THF (5 mL) was added. After 1 h, a solution of sodium azide (0.48 g, 7.49 mmol) in water (6 mL) was added to this solution. The reaction mixture was stirred at room temperature for 1 h. The reaction mixture was poured into ice water and extracted with EtOAc, dried over MgSO₄ and the solvent removed in vacuo. LC-MS of the crude product (0.9 g, 85%) indicated a mixture of acyl azide and isocyanate. The crude material (0.9 g, 2.1 mmol) was dissolved in THF (30 mL) and heated under reflux for 5 h. LC-MS Of the crude material indicated complete conversion of acyl azide to isocyanate and 1.5M HCl (10 mL) was added and heated at 60° C. for 2 h. THF was removed in vacuo, the aqueous layer was basified with saturated NaHCO₃ (sodium hydrogen carbonate) and extracted with DCM X2, dried over Na₂SO₄ (sodium sulphate) and the solvent removed in vacuo. The residue was purified by column chromatography eluting with DCM: Methanol (95:5). Yield: 500 mg (57%, from acid). Mass spectrum (ES-MS (+ve) 372 [M+H]⁺, Retention time 0.9 min. ¹H-NMR (CDCl₃, 400 MHz):δ 5.93 (1H, s, C₄—H), 3.27 (1H, m, NCH), 3.05(1H, d, C₁₁—H), 2.80 (1H, m, alkyl-H), 2.60-1.65 (18H, m, alkyl —H and NH₂), 1.47 (3H, s, Me), 1.43 (1H, m, alkyl —H), 1.02 (3H, s, Me).

### Example 22

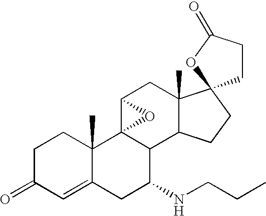

Intermediate 22-1 (0.1 g, 0.26 mmol) was dissolved in DCE (1,2-dichloroethane, 10 mL). Propanal (0.017 g, 0.29 mmol) and STAB (sodium triacetoxyborohydride, 0.085 g, 0.4 mmol) were added. The reaction mixture was stirred at room temperature for 16 h. The reaction mixture was diluted with EtOAc, the organic layer was washed with saturated NaHCO₃, dried over MgSO₄ and the solvent removed in vacuo. The residue was purified by column chromatography eluting with DCM:Methanol (97:3). Yield: 15 mg (14%). Mass spectrum (ES-MS (+ve) 414 [M+H]⁺, Retention time 0.94 min. ¹H-NMR (CDCl₃, 400 MHz):δ 5.90 (1H, s, C₄—H), 3.05(1H, d, C₁₁—H), 3.02 (1H, m, NCH), 2.80 (1H, m, alkyl-H), 2.70-1.60 (19H, m, alkyl —H and NH), 1.47 (3H, s, Me), 1.4(3H, m, alkyl —H), 1.02 (3H, s, Me), 0.9 (3H, t, Me).

### Example 23

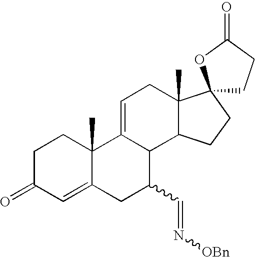

The compound of Example 6 (Aldehyde D; 3:1 C-7 alpha/beta, 100 mg, 270 μmol) was dissolved in DCM (1 mL). Benzyloxyamine (previously free based with aqueous potassium carbonate and extracted into ether, 27 mg, 270 μmol) was added and the mixture stirred at room temperature for 1 h. Starting material was consumed and the residue resulting from evaporation was purified by HPLC to give the title compound as a colourless gum (mixture of C7 α/β and E/Z diastereomers). Yield: 34 mg (27%). Mass spectrum (ES-MS (+ve)) 474 [M+H]⁺, Retention time 1.56 min, 100% UV. ¹H-NMR (CDCl₃, 400 MHz):δ 7.34 (5H, m, Ph), 7.07, 6.69, 6.31 (1H, d, oxime CH), 5.82, 5.77, 5.76, 5.72 (1H, s, C₄—H), 5.66 (1H, dd, C₁₁—H), 5.17-5.01 (2H, 4×s, PhCH₂), 2.93-1.56 (18H, m), 1.41, 1.37 (3H, 2×s, Me), 1.28 (1H, m), 0.91 (3H, s, Me).

### Example 24

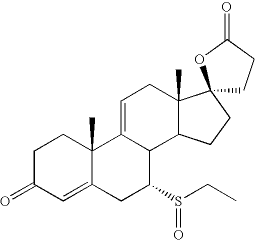

The compound of Example 13 (136 mg, 0.34 mmol) was dissolved in dichloromethane (10 mL) and cooled to −78° C. m-CPBA (1 eq. 77% pure, 76 mg) was added and the reaction was allowed to warm to room temperature overnight. The reaction mixture was diluted with dichloromethane and washed with saturated aqueous sodium bicarbonate, dried over MgSO₄, filtered and the solvent removed under reduced pressure. The crude was purified by flash column chromatography using ethyl acetate:methanol (5:1) as the eluent. Yield: 18 mg (13%). Mass spectrum (ES-MS (+ve)) 417 [M+H]⁺, Retention time 1.16 min. ¹H-NMR (CDCl₃, 400 MHz):δ 5.85 (1H, d, C₁—H), 5.69 (1H, s, C₄—H), 3.10 (1H, m), 2.95-2.82 (2H, m), 2.68-1.85 (18H, m), 1.43 (3H, s, CH₃), 1.35 (3H, t, CH₃), 0.94 (3H, s, CH₃).

### Example 25

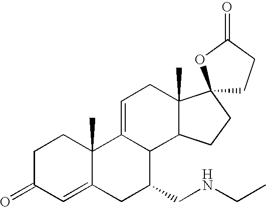

The compound of Example 6 (Aldehyde D; 3:1 C-7 alpha/beta, 100 mg, 271 μmol) was dissolved in dry DCM (2 mL) under nitrogen. 4A molecular sieves (500 mg) were added followed by ethylamine (2 M sol in THF, 122 μl, 244 μmol). The mixture was stirred at room temperature for 1 h and cooled on ice. Sodium triacetoxyborohydride (52 mg, 244 μmol) was added in 1 portion and the mixture stirred to room temperature over 1 h. The reaction mixture was quenched with methanol (0.1 mL), diluted with ethyl acetate (10 mL), extracted with 10% aqueous sodium bicarbonate (10 L), saturated brine (10 mL) and evaporated. The residue was purified by prep HPLC to give the title compound as a colourless gum (TFA salt, α-diastereomer). Yield: 21 mg (20%). Mass spectrum (ES-MS (+ve)) 398 [M+H]⁺, Retention time 1.05 min, 100% UV. ¹H-NMR (CDCl₃, 400 MHz): 5.97 (1H, s, C₄—H), 5.67 (1H, dd, C₁₁—H), 3.11-1.87 (21H, m), 1.56 (1H, m), 1.41 (3H, s, Me), 1.35 (1H, m), 1.26 (3H, t, Me), 0.93 (3H, s, Me), 0.88 (1H, m).

### Example 26

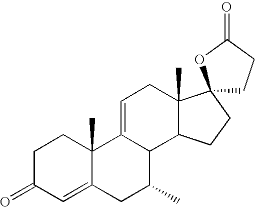

Copper I chloride (32 mg, 0.32 mmol) and lithium chloride (20 mg, 0.47 mmol) were stirred in a 1:1 mixture of diethyl ether:THF (9 mL) at room temperature for 30 min. The compound of Example 2 (Trienone A; 400 mg, 1.18 mmol) was added and stirring continued for 10 min. The reaction mixture was cooled to −30° C. and methylmagnesium bromide (3 M, 0.56 mL, 1.68 mmol) added over 20 min. Following a further 1.5 h at −30° C., the reaction was quenched with 5 N HCl (2.9 mL) at −30° C. Upon warming to room temperature, 2 N ammonia (7.5 mL) was added and the crude mixture was extracted with diethyl ether. The organics were washed with 0.5 N ammonia, water, dried over Na₂SO₄, filtered and the solvents removed under reduced pressure. The crude was purified by flash column chromatography using heptane:ethyl acetate (2:1 to 1:1) as eluent. Further purification of the product containing fractions by prep HPLC afforded the required 7 alpha product. Yield: 6.9 mg (2%). Mass spectrum (ES-MS (+ve)) 355 [M+H]⁺, Retention time 1.49 min. ¹H-NMR (CDCl₃, 400 MHz):δ 5.78 (1H, s, C₄—H), 5.65 (1H, d, C₁₁—H), 2.79-1.45 (19H, m), 1.37 (3H, s, CH₃), 0.94 (3H, s, CH₃), 0.66 (3H, m, CH₃).

### Example 27

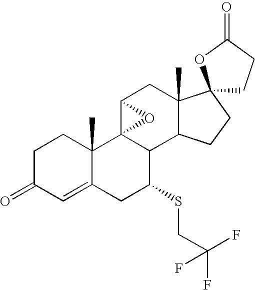

The compound of Example 1 (200 mg, 0.56 mmol) was suspended in 2,2,2-trifluoroethanethiol (2 mL) under nitrogen. The reaction mixture was heated to 30° C. Sodium metal (13 mg, 0.56 mmol) was added in 1 portion. The reaction was re-flushed with nitrogen, heated to 60° C. for 3 h and allowed to cool to ambient temperature. Saturated aqueous ammonium chloride (˜20 mL) was added and the aqueous phase extracted with dichloromethane. The organic layer was washed with saturated brine solution, dried over magnesium sulphate and the solvent evaporated in vacuo. The residue was purified by prep HPLC to give the required product. Yield: 50 mg (25%). Mass spectrum (ES-MS (+ve)) 471 [M+H]⁺, retention time 1.37 min. ¹H-NMR (CDCl₃, 400 MHz): [symbol] 5.9 (1H, s, C₄—H), 3.4(1H, s, CH), 3.1-1.7(19H, m, CH), 1.45 (3H, s, CH₃), 1.4 (2H, m, CH), 1.05 (3H, s, CH₃).

### Example 28

### Intermediate 28-1

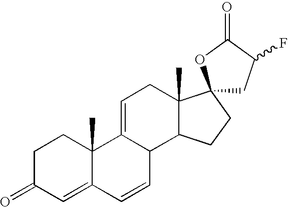

Intermediate 20-1 (613 mg, 1.65 mmol) and chloranil (384 mg, 1.56 mmol) were heated at 40° C. in DCM (4 mL), methanol (2.4 mL), and water (0.8 mL) for 2 h. The mixture was then quenched with 20% aqueous sodium metabisulfite (0.3 mL) and stirred at room temperature for 0.5 h, diluted with water (3 mL) and stood for 0.5 h. The mixture was extracted with DCM (2×5 mL), the organic layers combined and dried over sodium sulphate and filtered. The filtrate was allowed to stand over polymer supported-carbonate resin (1.1 g, 1.5 mmol/g) for 1 h, filtered and evaporated to give the title compound as a pale yellow foam. Yield: 523 mg (89%). Mass spectrum (ES-MS (+ve)) 357 [M+H]⁺, Retention time 1.42 min, 95% UV. ¹H-NMR (CDCl₃, 400 MHz): δ 6.17 (2H, m, C₆—H , C₇—H), 5.73 (1H, s, C₄—H), 5.56 (1H, 2×dd, C₁₁—H), 5.46-5.11 (1H, 2×dt, C₂₂—HF), 2.99 (1H, m), 2.72-1.42 (12H, m), 1.34, 1.27 (3H, 2×s, Me), 1.11 (1H, m), 1.08, 1.02 (3H, 2×s, Me). ¹⁹F-NMR (CDCl₃, 376 MHz): −190.58, −195.61.

### Example 28

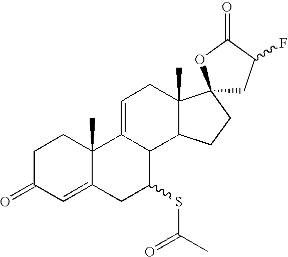

Intermediate 28-1 (92 mg, 258 μmol) was dissolved in methanol (2 mL) and thioacetic acid (0.5 mL) added. The mixture was heated at 60° C. for 3 h after which time ¹H-NMR analysis indicated consumption of the starting material. The mixture was evaporated and the residue purified by prep HPLC to give a yellow gum (47 mg, 42%). The gum was taken up in the minimum amount of methanol (ca 150 μl) and carefully precipitated by the addition of water. A pale yellow solid was obtained (20.5 mg) which was further purified by flash chromatography on silica eluting with heptane-ethyl acetate (4:1 to 1:1) to give the title compound as a pale yellow solid (mixture of four diastereomers). Yield: 17 mg (16%). Mass spectrum (ES-MS (+ve)) 433 [M+H]⁺, Retention time 1.45 min, 100% UV. The major mass ion observed was 357 [M+H−AcSH]⁺. ¹H-NMR (CDCl₃, 400 MHz):δ 0.78, 5.74 (1H, 2×s, C₄—H), 5.70, 5.66 (1H, 2×dd, C₁₁—H), 5.44-5.22 (1H, 2×dt, C₂₂—HF), 4.06 (1H, m), 3.00 (1H, dd), 2.84-2.39 (5H, m), 2.32 (3H, 4×s, SAc), 2.27-1.51 (10H, m), 1.40-0.86 (6H, m, 2×Me). ¹⁹F-NMR (CDCl₃, 376 MHz): −190.92, −191.13, 196.07, −196.42. (ratio 1:5:13:5).

### Example 29

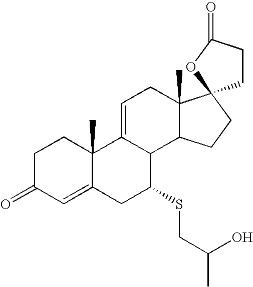

The compound of Example 2 (Trienone A; 200 mg, 0.6 mmol) was dissolved in tetrahydrofuran (3 mL) and 1-mercapto-2-propanol (63 μl, 15.4 mmol) added under nitrogen. The reaction mixture was heated to 30° C. Sodium metal (33 mg, 1.44 mmol) was added in one portion and the reaction was re-flushed with nitrogen. The reaction mixture was heated to 60° C. for 4 h and stirred for 15 h at ambient temperature. 1-Mercapto-2-propanol (0.5 mL) was added, the reaction re-flushed with nitrogen and heated to 60° C. for a further 3.5 h. The reaction was allowed to cool and stirred at ambient temperature for 40 h. Saturated aqueous ammonium chloride was added and the aqueous phase extracted with dichloromethane. The organic layer was washed with saturated brine solution, dried over magnesium sulphate and the solvent evaporated in vacuo. The residue was purified by flash chromatography using 2:1 ethyl acetate:heptane as eluent. Further purification by prep HPLC gave the required product. Yield: 100 mg (39%). Mass spectrum (ES-MS (+ve)) 431 [M+H]⁺, retention time 1.84 min. ¹H-NMR (CDCl₃, 400 MHz): δ 5.8 (1H, s, C₄—H), 5.6 (1H, d, C₁₁—H), 3.8 (1H, d, OH), 3.2 (1H, s, CH), 2.9 (1H, m, CH), 2.7-1.8 (19H, m, CH), 1.45 (1H, m, CH), 1.35 (3H, s, CH₃), 1.25 (3H, d, CH₃), 0.9 (3H, s, CH₃)

### Example 30

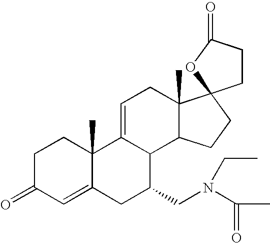

The crude amine from Example 25 (80 mg, 0.20 mmol) was dissolved in DCM (10 mL) and triethyl amine (0.03 g, 0.3 mmol) added. To this mixture acetyl chloride (0.018 g, 0.24 mmol) was added drop wise. The reaction mixture was stirred at room temperature for 16 h. The reaction mixture was diluted with DCM, the organic layer washed with saturated NaHCO₃, dried over MgSO₄ and the solvent removed in vacuo. The residue was purified by flash column chromatography eluting with DCM:Methanol (97:3). Yield: 29 mg (30%). Mass spectrum (ES-MS (+ve) 440 [M+H]⁺, Retention time 1.85 min. ¹H-NMR (CDCl₃, 400 MHz):(rotamers observed) δ 5.75 (1H, s, C₄—H), 5.65 (1H, d, C₁₁—H), 3.70(1H, m, alkyl-H), 3.50-3.00 (3H, m, alkyl-H), 2.65-1.75 (18H, m, alkyl-H ), 2.06 (3H, s, COMe), 1.40 (1H, m, alkyl-H), 1.35 (3H, s, Me), 1.15 (3H, t, Me), 0.80 (3H, s, Me).

### Example 31

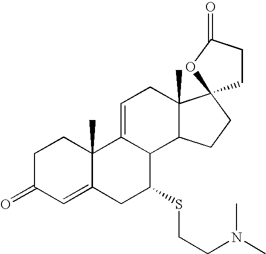

The compound of Example 2 (Trienone A; 250 mg, 0.74 mmol) was dissolved in methanol (3 mL) under nitrogen. Dimethylaminoethanethiol hydrochloride (136 mg, 0.96 mmol) and indium(III) chloride (16.3 mg, 0.07 mmol) were added and the reaction stirred at ambient temperature overnight. The reaction mixture was diluted with diethyl ether (20 mL) and washed with water (2×20 mL). The aqueous layer was basified with aqueous saturated sodium bicarbonate to pH 12 and extracted with ethyl acetate. The organic phase was dried over magnesium sulphate and the solvent evaporated in vacuo. The residue was purified by prep HPLC and further purified by flash column chromatography using 5-10% Methanol in DCM as eluent yielding the required product. Yield: 20 mg (8%). Mass spectrum (ES-MS (+ve)) 444 [M+H]⁺, retention time 1.16 min. ¹H-NMR (CDCl₃, 400 MHz):δ 5.8 (1H, s, C₄—H), 5.65 (1H, d, C₁—H), 3.3 (1H, d, CH), 3.2-1.8 H, m, CH), 1.5 (1H, m, CH), 1.4 (3H, s, CH₃), 0.95 (3H, s, CH₃).

### Example 32

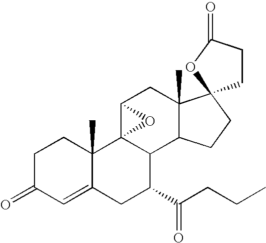

To the compound of Example 3 (150 mg, 0.375 mmol) in dichloromethane (5 mL) at 0° C. under nitrogen was added 1-chloro-N,N-2-trimethyl-1-propenylamine (0.06 mL, 0.45 mmol). The reaction was stirred at 0° C. for 1 h; the solvent was removed under reduced pressure and the crude acid chloride was re-dissolved in THF (3 mL). In a separate flask, zinc chloride (56 mg, 0.41 mmol) was heated under nitrogen flow to remove traces of moisture and allowed to cool to room temperature. THF (5 mL) was added and the mixture cooled to 0° C. and n-propylmagnesium bromide (2 M, 0.41 mL, 0.83 mmol) added. The mixture was stirred at 0° C. for 1 h and Pd(PPh₃)₄ (cat. 3 mg) was added. After a further 5 min stirring, the acid chloride was added over 5 min. The mixture was allowed to warm to room temperature overnight. The solvent was removed under reduced pressure and the crude mixture was dissolved in dichloromethane. The organic layer was washed with water and the solvent was removed under reduced pressure. The required ketone was purified by prep HPLC. Yield: 33 mg (21%). Mass spectrum (ES-MS (+ve)) 427 [M+H]⁺, Retention time 1.35 min. ¹H-NMR (CDCl₃, 400 MHz):δ 5.93 (1H, s, C₄—H), 3.14 (1H, d, C₁₁—H), 2.90-2.80 (2H, m), 2.68-1.60 (19H, m), 1.53 (3H, s, CH₃), 1.51-1.43 (2H, m), 1.01 (3H, s, CH₃), 0.87 (3H, t, CH₃)

### Example 33

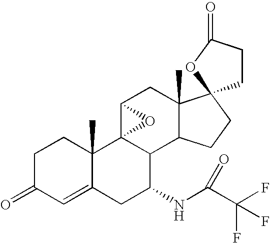

Intermediate 22-1 (54.7 mg, 0.147 mmol) was dissolved in DCM (5 mL) and pyridine (0.013 mL, 0.155 mmol) added. The reaction was cooled to 5° C., to this mixture trifluroacetic anhydride (0.022 mL, 0.155 mmol) was added dropwise. The reaction mixture was stirred at 0-5° C. for 1 h. The reaction mixture was diluted with DCM, the organic layer was washed with saturated NaHCO₃, 0.5M HCl, water, saturated brine, dried over Na₂SO₄ and the solvent removed in vacuo. The residue was 96% pure and was not further purified. Yield: 62 mg (91%). Mass spectrum (ES-MS (+ve) 468 [M+H]⁺, Retention time 1.39 min. ¹H-NMR (CDCl₃, 400 MHz): δ 7.05 (1H, d, NH), 5.85 (1H, s, C₄—H), 4.35 (1H, m, NCH), 3.15 (1H, d, C₁₁—H), 2.80-1.45 (17H, m, alkyl-H), 1.41 (3H, s, Me), 1.35 (1H, m, alkyl-H), 0.95 (3H, s, Me).

### Example 34

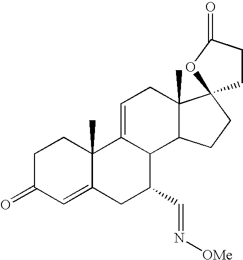

The 7α-Aldehyde E of Example 6 (16 mg, 43 μmol) was dissolved in anhydrous DCM (2 mL). Methoxyamine hydrochloride (3.1 mg, 37 μmol) and potassium carbonate (5.9 mg, 43 μmol) were added and the mixture stirred at room temperature for 2 h. After evaporation the residue was purified by prep HPLC to give the title compound as a colourless gum. Yield: 4.5 mg (26%) (7 alpha). Mass spectrum (ES-MS (+ve)) 398 [M+H]⁺, Retention time 1.05 min, 100% UV. ¹H-NMR (CDCl₃, 400 MHz):δ 6.99 (1H, d, oxime CH), 5.68 (1H, s, C₄—H), 5.41 (1H, dd, C₁₁—H), 3.58 (3H, s, OMe), 2.92 (1H, dd, C₈—H), 2.80 (1H, m, C₇—H), 2.67-1.58 (16H, m), 1.44 (3H, s, Me), 1.41 (1H, m), 0.96 (3H, s, Me).

### Example 35

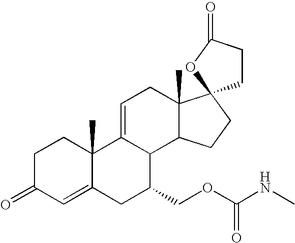

The compound of Example 36 (3:1 mixture of diastereomers, 75 mg, 202 μmol) was dissolved in chloroform (2 mL). Methyl isocyanate (20 fold excess, 200 mg) was added and the mixture heated at 50° C. for 72 h. LCMS showed 50% conversion and the mixture was evaporated and purified by prep HPLC to give the 7 alpha-diastereomer as a colourless solid. Yield: 19 mg (22%). Mass spectrum (ES-MS (+ve)) 428 [M+H]⁺, Retention time 1.31 min, 100% UV. ¹H-NMR (CDCl₃, 400 MHz): δ 5.77 (1H, s, C₄—H), 5.54 (1H, dd, C₁—H), 4.47 (1H, br s, NH), 4.15 (1H, dd), 3.37 (1H, dd), 2.79 (3H, d, NMe), 2.70-1.44 (17H, m), 1.41 (3H, s, Me), 1.31 (1H, m), 0.93 (3H, s, Me), 0.91 (1H, m).

### Example 36

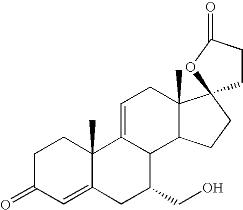

The 7α-Aldehyde D of Example 6 (3:1 C-7 alpha/beta, 750 mg, 2 mmol) was dissolved in dry THF (15 mL) under nitrogen. Lithium tri-tert-butoxyaluminiun hydride (1.1 M in THF, 0.9 mL) was added and the mixture stirred at room temperature for 1 h. The reaction mixture was quenched by the addition of glacial acetic acid to pH ˜4 and evaporated. The residue was taken up in ethyl acetate (20 mL) and extracted with water (20 mL), saturated brine (20 mL), dried over sodium sulfate and evaporated. The pale yellow residue was purified by chromatography on silica eluting with ethyl acetate to give the title compound. The 7α-diastereomer was separated by prep HPLC. Yield: 37 mg (5%). Mass spectrum (ES-MS (+ve)) 371 [M+H]⁺, Retention time 1.30 min. ¹H-NMR (d₆-acetone, 400 MHz): δ 5.54 (1H, s, C₄—H), 5.43 (1H, dd, C₁₁—H), 3.48 (2H, m, CH₂OH), 3.12-1.26 (20H, m), 1.24 (3H, s, Me) 0.79 (3H, s, Me).

### Example 37

### Intermediate 37-1: 11 β-Acetoxy-17-hydroxyimino-3-methoxy 3,5-androsta-diene

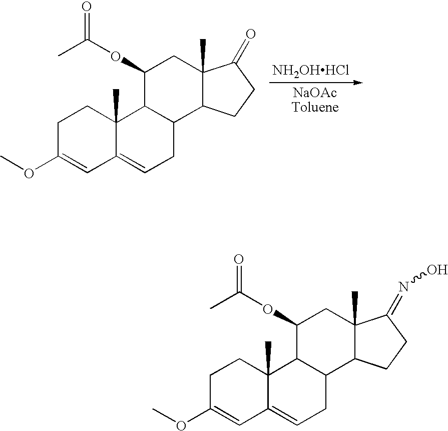

To a solution of the 11β-Acetoxy-17-KETO-3-methoxy 3,5-androstadiene (100 mg, 0.28 mmol; prepared using the method for compound S6 (WO 97/217290) from 11-62 acetoxy androsten-4-ene-3,17-dione; the latter prepared from hydrocortisone as described in Steroids vol 68(2), 2003, 139-142) in toluene (10 mL) was added hydroxylamine hydrochloride (77 mg, 1.12 mmol), anhydrous sodium acetate (110 mg, 1.39 mmol) and the resulting suspension heated at 95° C. for 1 h. After cooling, the reaction mixture was diluted with ethylacetate; the organic solution was washed once with water, dried over MgSO₄ and concentrated under reduced pressure to give the product as a white solid. ES-MS m/z 374 (M+H⁺).

### Intermediate 37-2: 11-β-acetoxy-17-nitro-4-androsten-3-one

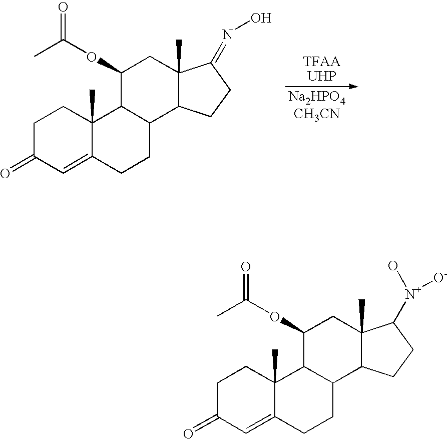

A suspension of urea hydrogen peroxide (0.056 g, 0.60 mmol) in acetonitrile (0.50 mL) was stirred and cooled in an ice bath. To this was added a solution of trifluoroacetic anhydride (0.07 mL, 0.50 mmol) in acetonitrile (0.50 mL) dropwise over a period of 10 min. The resulting solution was left to stir at 0° C. for 30 min. Meanwhile a solution of the Intermediate 33-1 (0.090 g, 0.25 mmol) in acetonitrile (1.0 mL) was stirred and disodium hydrogen phosphate (0.20 g, 1.39 mmol) added. The suspension was heated to 80° C. and the pre-prepared pertrifluoroacetic acid solution added dropwise over 15 min. Heating was continued for a further 30 min and the reaction allowed to cool to room temperature. The reaction mixture was diluted with ethyl acetate, washed with sodium bicarbonate (aq) followed by saturated brine. The solution was dried over magnesium sulphate and concentrated under reduced pressure. The residue was purified using a silica SPE cartridge eluting with hexane/ethyl acetate 9:1→1:2 giving the desired product. ¹H NMR (CDCl₃) δ 5.70 (s, 1H), 5.45 (m, 1H), 4.35 (m, 1H), 2.05 (s, 3H), 1.30 (s, 3H), 0.90(s, 3H); ES-MS m/z 376 (M+H⁺).

### Intermediate 37-3: 11-β-Acetoxy-17-spirolactam-4-androsten-3-one

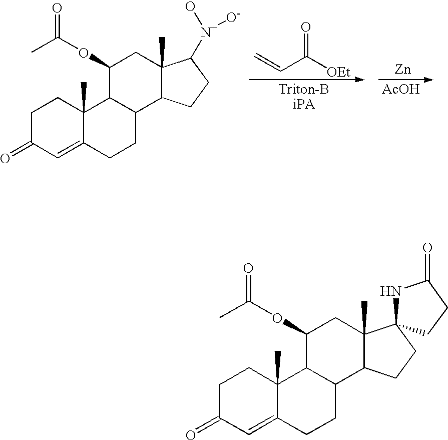

To a solution of Intermediate 33-2 (0.40 g, 1.07 mmol) in isopropanol (3 mL) was added ethyl acrylate (5.5 mL, excess) and Triton-B (0.71 mL, 1.56 mmol). The dark solution was stirred at room temperature for 4 h and poured onto ice/water. The stirred mixture was neutralised with dilute HCl (aq) and extracted into 2 portions of ethyl acetate. The combined extracts were washed with brine, dried over magnesium sulphate and concentrated under reduced pressure giving a yellow gum. (high vacuum pump was required in order to remove the excess ethyl acrylate). To a solution of the yellow gum in acetic acid (30 mL) was added zinc powder (0.5 g, excess). The reaction was stirred and heated to 110° C. for a period of 6 h, and then cooled and concentrated under reduced pressure. The residue was taken into ethyl acetate; the solution was washed once with water and once with sodium bicarbonate, dried over magnesium sulphate and concentrated under reduced pressure. The material was purified using silica chromatography; methanol was required in order to elute the product, a poor separation was obtained. Preparative HPLC removed a number of impurities and then a second silica column eluting with 19:1 EtOAc/MeOH gave the desired product. ¹H NMR (CDCl₃) δ 6.00 (br s, 1H), 5.75 (s, 1H), 5.45 (m, 1H), 2.10 (s, 3H), 1.30 (s, 3H), 1.05(s, 3H); ES-MS m/z 400 (M+H⁺).

### Intermediate 37-4: 11-β-Hydroxy-17-spirolactam-4-androsten-3-one

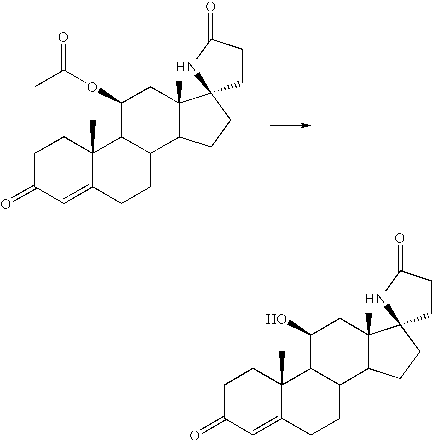

To a solution of Intermediate 33-3 (61 mg, 0.152 mmol) in methanol (2 mL) was added lithium hydroxide (26 mg, 0.61 mmol), and the reaction was stirred at room temperature for 72 h. The solvent was removed in vacuo, and the product was re-dissolved in 2M HCl, and extracted into ethyl acetate. The organic layer was separated, dried over MgSO₄ and reduced in vacuo to give the product as a white solid.

### Example 37

### 17-Spirolactam-4,9(11)-androstant-dien-3-one

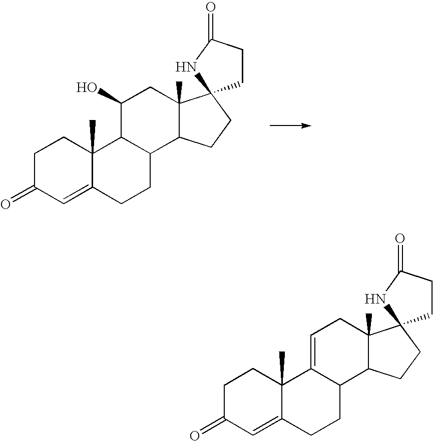

The steroid alcohol (16 mg, 0.045 mmol) was dissolved in a mixture of toluene (0.15 mL), water (0.05 mL) and conc. hydrochloric acid (0.05 mL), and the solution was heated at 85° C. for 4 h. The reaction was allowed to cool, diluted with ethyl acetate, and then washed with a 2 M solution of sodium carbonate. The organic layer was separated, dried over MgSO₄ and reduced in vacuo. The product was purified by prep-HPLC to give the product as a colourless solid. ¹H NMR (CDCl₃) δ 5.89 (br s, 1H), 5.78 (s, 1H), 5.58 (d, 1H), 1.35 (s, 3H), 0.82 (s, 3H).

### Example 38

### Intermediate 38-1: 17-beta-20-dihydroxy 9(11)-ene 4-androstene-3-one

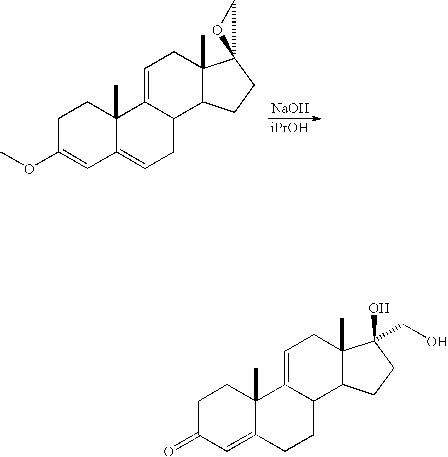

A solution of the compound of Example 4 (oxirane B) (0.940 g, 2.88 mmol) in a mixture of 3.5M solution of sodium hydroxide (30 mL) and isopropanol (60 mL) was stirred at 80° C. for 16 h. After cooling, the isopropanol was evaporated and the mixture neutralised with concentrated HCl. Then, the crude mixture was extracted with ethyl acetate (2×100 mL) and dried over Na₂SO₄ before concentrating to dryness under vacuum to the product as a yellow solid. ¹H NMR (CDCl₃) δ 5.77 (bs, 1H), 5.56 (m, 1H), 3.73 (d, J=10.8 Hz, 1H), 3.43 (d, J=10.8 Hz, 1H), 2.64-1.2 (m, 16H), 1.36 (s, 3H), 0.91 (s, 3H). ES-MS m/z 317 (M+H⁺).

### Example 38

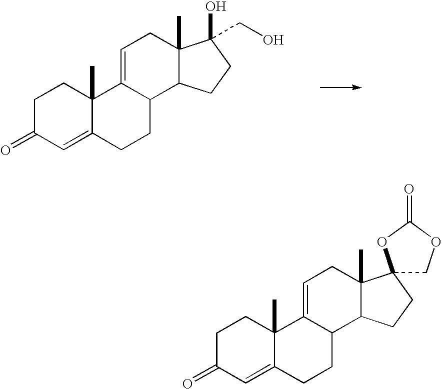

To a solution of Intermediate 38-1 (0.98 g, 3.11 mmol) in dichloromethane (50 mL) was added diphosgene (0.45 mL, 3.73 mmol) and pyridine (0.61 mL, 7.46 mmol). The reaction was stirred at rt for 1 h, and then washed with 2M HCl (20 mL). The organic layer was separated and reduced in vacuo, before purifying by silica column chromatography, eluting with EtOAc/hexane (2:1) to give a pale yellow solid. ¹H NMR (CDCl₃) δ 5.76 (s, 1H), 5.58 (s, 1(s, 3H), 4.3 (dd, 2H), 1.37 (s, 3H). ES-MS m/z 343 (M+H⁺).

### Example 39

### Intermediate 39-1

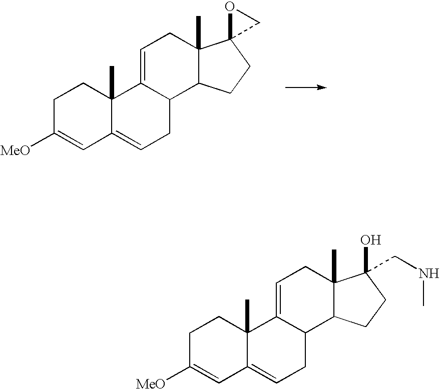

To a solution of the compound of Example 4 (Oxirane B) (1.0 g, 3.2 mmol) in ethanol (20 mL) was added methylamine (10 mL, ˜50% solution in water) and acetic acid (300 μL). The mixture was heated for 18 h at 45° C. with stirring, after which the reaction was cooled, diluted with water and extracted into ethyl acetate (2×100 mL). The organic layers were dried over MgSO₄, filtered, and the solvent removed under reduced pressure to give the product as a brown solid, which was used crude for the next reaction.

### Example 39

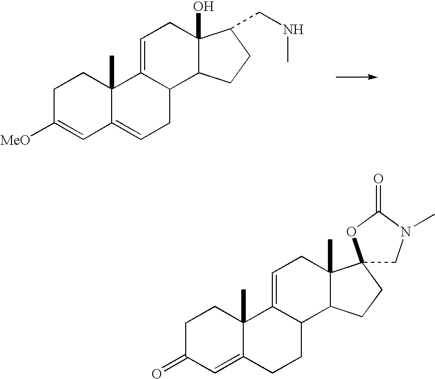

To a solution of the steroid amine (3.2 mmol) in dichloromethane (60 mL) was added triphosgene (1.61 g, 5.44 mmol) and pyridine (0.88 mL, 10.9 mmol). The reaction was stirred at rt for 1.5 h, and then washed with 2M HCl (60 mL). The organic layer was separated and reduced in vacuo, before purifying by silica column chromatography, eluting with EtOAc/hexane (1:1) to give an off-white solid. ¹H NMR (CDCl₃) δ 5.76 (s, 1H), 5.57 (s, 1H), 3.4 (dd, 2H), 2.87 (s, 3H), 1.37 (s, 3H), 0.96 (s, 3H). ES-MS m/z 356 (M+H⁺).

### Example 40

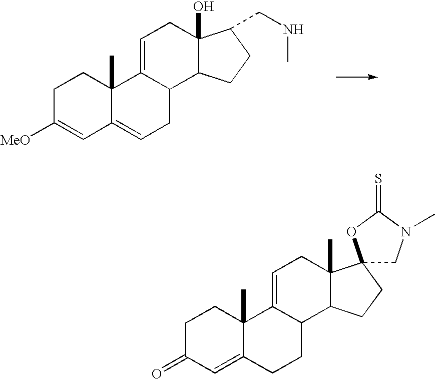

To a solution of Intermediate 39-1 (450 mg, 1.31 mmol) in dichloromethane (30 mL) was added thiophosgene (195 μL, 2.6 mmol) and pyridine (425 μL, 5.2 mmol). The reaction was stirred at rt for 1.5 h, and then washed with 2M HCl (20 mL). The organic layer was separated and reduced in vacuo, before purifying by silica column chromatography, eluting with EtOAc/hexane (1:1) to give a pale yellow solid. ¹H NMR (CDCl₃) δ 5.77 (s, 1H), 5.57 (d, 1H), 3.6 (dd, 2H), 3.22 (s, 3H), 1.37 (s, 3H), 1.02 (s, 3H). ES-MS m/z 372 (M+H⁺).

### Example 41

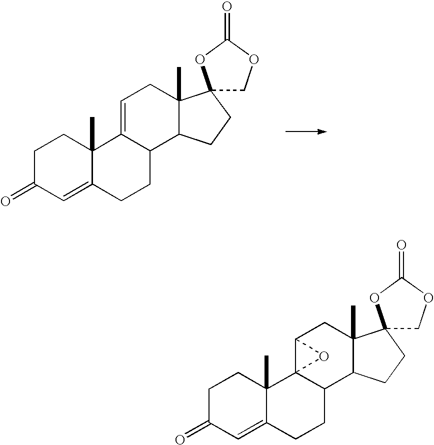

To a solution of the compound of Example 38 (0.35 g, 1.02 mmol) in dichloromethane (15 mL) at −10° C. was added 3-chloroperbenzoic acid (0.61 g, 1.75 mmol) in dichloromethane (10 mL), and the reaction was stirred at −10° C. for 1 h, then brought to rt and stirred for 2 h. The reaction mixture was diluted with dichloromethane and washed with saturated Na₂S₂O₅ solution, saturated NaHCO₃ solution and water, and the organic layer was dried over MgSO₄ before reducing in vacuo to give the epoxy-steroid as a white solid. This product was purified by silica column chromatography, eluting with EtOAc/hexane (2:1) to give a white solid. ¹H NMR (CDCl₃) δ 5.85 (s, 1H), 4.3 (dd, 2H), 3.27 (s, 1H), 1.48 (s, 3H), 1.04 (s, 3H). ES-MS m/z 359 (M+H⁺).

### Example 42

### Intermediate 42-1

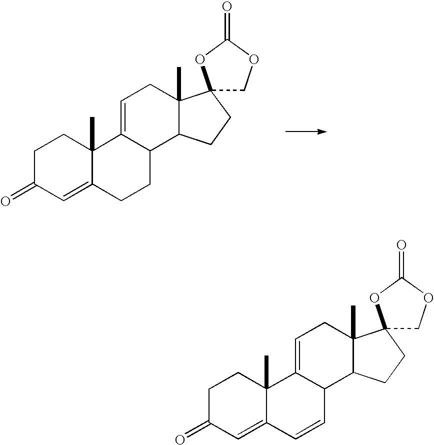

A stirred solution of the compound of Example 38 (70 mg, 0.2 mmol) and DDQ (50 mg, 0.22 mmol) in 1,4-dioxane (7 mL) was treated with a solution of 2M hydrogen chloride in ether (2.45 mL). The resulting solution was stirred at room temperature for 30 min, after which time a yellow solid had precipitated from the reaction mixture. The reaction mixture was filtered, and diluted with CH₂Cl₂ (20 mL) and NaHCO₃ (1% aqueous solution, 20 mL) was added. The two layers were separated, and the organic phase washed with a further portion of NaHCO₃ (1% aqueous solution, 20 mL). The organic phase was dried over MgSO₄, filtered, and the solvent removed under reduced pressure to give the product as an orange gum.

### Example 42

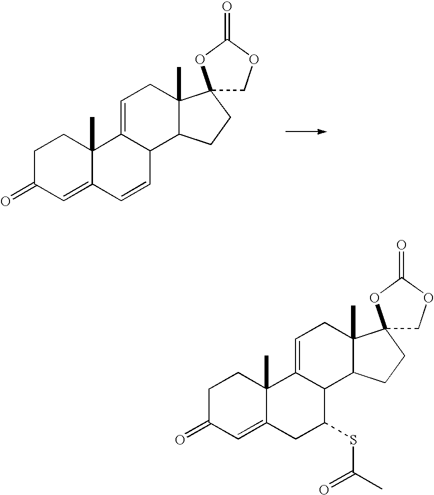

To a solution of Intermediate 42-1 (70 mg, 0.20 mmol) in anhydrous THF (2 mL) was added thiolacetic acid (44 μL, 0.62 mmol) and TMSOTf (19 μL, 0.13 mmol). The reaction mixture was heated at 45° C. for 5 h, diluted with ethyl acetate and washed with saturated NaHCO₃ solution. The organic phase was dried over MgSO₄, filtered, and the solvent removed under reduced pressure to give the product as a yellow solid. This crude product was purified by prep-HPLC to give the product as a colourless solid. ¹H NMR (CDCl₃) δ 5.75 (s, 1H), 5.70 (s, 1H), 4.3 (dd, 2H), 4.07 (q, 1H), 1.40 (s, 3H), 0.99 (s, 3H). ES-MS m/z 341 ([M+H−(HSCOCH₃)]⁺), 417 (M+H⁺).

### Example 43

### Intermediate 43-1

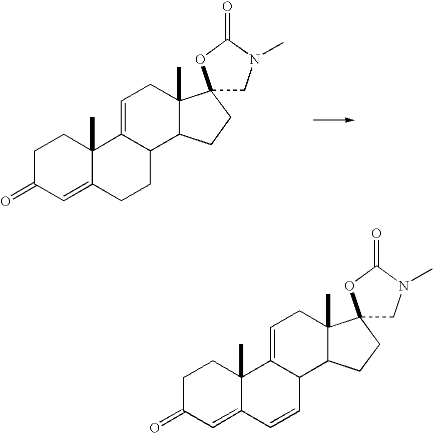

A stirred solution of the compound of Example 39 (150 mg, 0.42 mmol) and DDQ (103 mg, 0.46 mmol) in 1,4-dioxane (15 mL) was treated with a solution of 2M hydrogen chloride in ether (5.1 mL). The resulting solution was stirred at room temperature for 30 min, after which time a yellow solid had precipitated from the reaction mixture. The reaction mixture was filtered, and diluted with CH₂Cl₂ (40 mL) and NaHCO₃ (1% aqueous solution, 40 mL) was added. The two layers were separated, and the organic phase washed with a further portion of NaHCO₃ (1% aqueous solution, 40 mL). The organic phase was dried over MgSO₄, filtered, and the solvent removed under reduced pressure to give the product as a yellow oil.

### Example 43

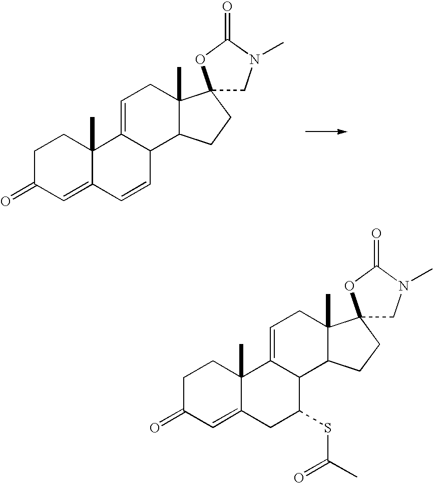

To a solution of Intermediate 43-1 (0.42 mmol) in anhydrous THF (5 mL) was added thiolacetic acid (100 μL, 1.33 mmol) and TMSOTf (44 μL, 0.21 mmol). The reaction mixture was heated at 50° C. for 5 h, diluted with ethyl acetate and washed with saturated NaHCO₃ solution. The organic phase was dried over MgSO₄, filtered, and the solvent removed under reduced pressure to give the product as a yellow solid. This crude product was purified by prep-HPLC to give the product as a colourless solid. ¹H NMR (CDCl₃) δ 5.75 (s, 1H), 5.69 (d, 1H), 4.08 (m, 1H), 3.4 (dd, 2H), 2.88 (s, 3H), 2.33 (s, 3H), 1.37 (s, 3H), 0.98 (s, 3H). ES-MS m/z 430 (M+H⁺).

### Example 44

### Intermediate 44-1

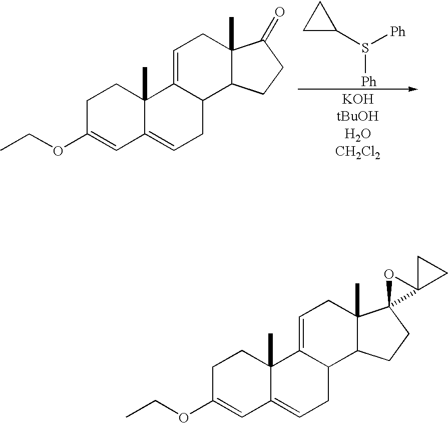

Potassium hydroxide (0.09 g, 1.6 mmol) was added to a solution of 3 ethoxy-3,5,9(111)-androstan-trien-17-one (0.200 g, 0.64 mmol; prepared as in WO 97/21720 from commercially available 4,9(11)-androsatdien-3,17-dione) and tetrafluoroboro cyclopropyldiphenyl sulfonium (0.2 g, 0.64 mmol) in a mixture of dichloromethane (5 mL), tert-butanol (0.04 mL) and water (0.018 mL) and the resulting mixture was stirred at r.t. for 2 h. Then, it was heated at reflux for 16 h. After cooling, the reaction was diluted with more dichloromethane (20 mL) and poured over water (20 mL). The organic phase was separated and dried over Na₂SO₄ before concentrating to dryness under vacuum to give dark thick oil.

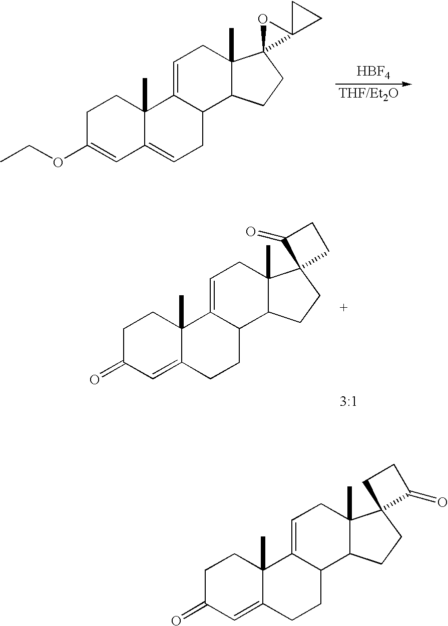

Tetrafluoroboric acid (2 mL) was added to a solution of Intermediate 44-1 (0.64 mmol) in a mixture of tetrahydrofuran (2 mL) and ethyl ether (2 mL) and the resulting mixture was stirred at r.t. for 2 h. The reaction was diluted with more ethyl acetate (10 mL) and washed with 2M solution of sodium carbonate (2×10 mL). The organic phase was dried over MgSO₄ before concentrating to dryness under vacuum to give dark solid. The compound was purified by prep-HPLC to give 47 mg of a 3:1 mixture of cyclobutanones. The mixture was separated by prep HPLC to isolate the compound of Example 44 as a white solid.

### Example 44

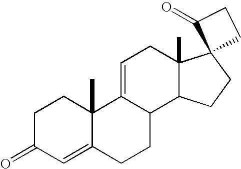

¹H NMR (CDCl₃). δ 5.77 (s, 1H), 5.58 (d, J=6.2 Hz, 1H), 2.83 (m, 2H), 2.64-1.2 (m, 18H), 1.40 (s, 3H), 0.88 (s, 3H). ES-MS m/z 325 (M+H⁺).

### Example 45

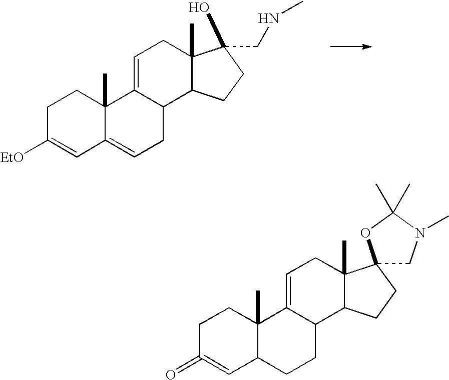

The 3-ethoxy derivative of Intermediate 39-1 (350 mg, 0.91 mmol) was dissolved in ethanol (10 mL) and to this was added conc. HCl (4 drops) with stirring. After 30 min, aqueous sodium bicarbonate and dichloromethane were added, the organic layer was separated and reduced in vacuo. The resulting oil was dissolved in acetone and left for 24 h, before being reduced in vacuo to give 17-spiro-(2,2,3-trimethyloxazolidine)-4,9(11)-androstadien-3-one as a colourless oil. ¹H NMR (CDCl₃) δ 5.66 (s, 1H), 5.55 (d, 1H), 2.98 (d, 1H), 2.44 (d, 1H), 2.17 (s, 3H), 1.30 (s, 3H), 1.12 (s, 3H), 1.06 (s, 3H), 0.76 (s, 3H).

### Example 46

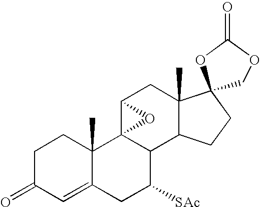

To a solution of the compound of Example 42 (0.35 g, 1.02 mmol) in dichloromethane (15 mL) at −10° C. was added 3-chloroperbenzoic acid (0.61 g, 1.75 mmol) in dichloromethane (10 mL), and the reaction was stirred at −10° C. for 1 h, then brought to rt and stirred for 2 h. The reaction mixture was diluted with dichloromethane and washed with saturated Na₂S₂O₅ solution, saturated NaHCO₃ solution and water, and the organic layer was dried over MgSO₄ before reducing in vacuo to give the epoxy-steroid as a white solid. This product was purified by silica column chromatography, eluting with EtOAc/hexane (2:1) to give a white solid. ¹H NMR (CDCl₃) δ 5.75 (s, 1H), 4.3 (dd, 2H), 4.07 (q, 1H), 2.34 (s, 3H) 1.40 (s, 3H), 0.99 (s, 3H). ES-MS m/z 357 ([M+H—(HSCOCH₃)]⁺), 433 (M+H⁺).

### Example 47

### Intermediate 47-1

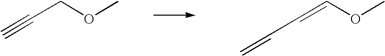

Potassium tert-butoxide (0.57 g) was added to sieve-dried methyl propargyl ether (7 g, 0.1 mmol), and the mixture was heated at reflux for 3 h. It was then distilled under reduced pressure at r.t. to a cardice-cooled trap to give a colourless liquid (6.4 g, 91% yield) of 3-methoxy-1,2-propadiene ¹H NMR (CDCl₃) δ 6.50 (t, 1H), 5.45 (d, 2H), 3.39(s, 3H); boiling point 50-52° C.

### Example 47

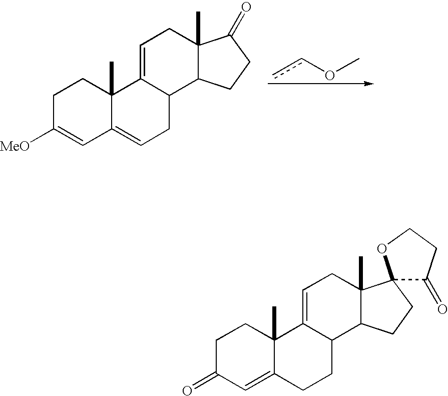

To a stirred solution of n-butyllithium (1.6 M in hexanes, 6.5 mL, 10.4 mmol) in THF (5 mL) at −78° C. under nitrogen was added 3-methoxy-1,2-propadiene (700 mg, 10.0 mmol). The mixture was stirred for 30 min, after which the 17-keto-3-methoxy-3,5-androstadiene (1.0 g, 3.35 mmol, prepared as described in WO 97/21720) in THF (2 mL) was added. After stirring for 3 h at −78° C. the reaction was quenched with saturated aqueous ammonium chloride solution, and the adduct was extracted into DCM, dried over MgSO₄, and concentrated in vacuo.

The crude adduct was re-dissolved in tert-butanol (7.5 mL) and to this was added potassium tert-butoxide (1.50 g, 13.5 mmol) and 18-crown-6 (50 mg), and the mixture was heated at reflux for 4 h. The reaction was quenched with 6M HCl (5 mL), and the product was extracted into DCM and dried over MgSO₄. The crude product was then purified by chromatography on silica gel, eluting with 5:1 petroleum ether/ethyl acetate, to give the pure product (100 mg, 9% yield) as an orange solid. ¹H NMR (CDCl₃) δ 5.77(s, 1H), 5.47(d, 1H), 4.16(m, 2H), 1.39(s, 3H), 0.99 (s, 3H). LC-MS m/z 341 (MH⁺), retention time 4.38 (method A).

### Example 48

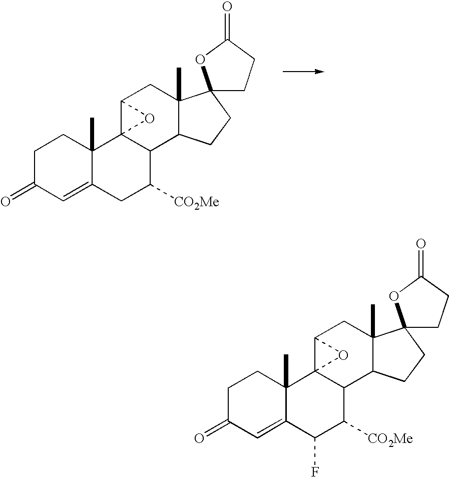

To a solution of eplerenone (238 mg) in acetic anhydride (1.0 mL) was added acetyl chloride (0.5 mL). The reaction was heated at 60° C. for 2.5 h, and then poured onto a saturated solution of sodium bicarbonate in water. The 3-acetyl protected product was extracted into ethyl acetate, dried over MgSO₄ and reduced in vacuo to give 200 mg (0.44 mmol) of a pale powder.

This product was dissolved in acetonitrile (5 mL), and to this solution was added Selectfluor™ (156 mg, 0.44 mmol), methanesulphonic acid (30 μL, 0.44 mmol) and pyridine (40 μL, 0.5 mol), and mixture was stirred at room temperature for 3 h. Water and dichloromethane were then added, and the organic layer was separated, reduced in vacuo and purified by prep-HPLC to give 40 mg of a pale powder. ¹H NMR (CDCl₃) δ 6.06 (d, 1H), 5.42 (dd, 1H), 3.65 (s, 3H), 3.16 (m, 1H), 2.74 (m, 1H), 0.94 (s, 3H); ES-MS m/z 433 (M+H⁺).

### Example 49

### Intermediate 49-1

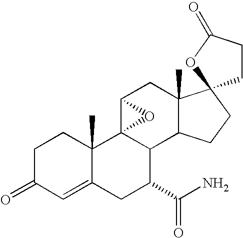

The compound of Example 3 (100 mg, 0.025 mmol) was dissolved in DCM (dichloromethane, 10 mL) and cooled 0° C., 1-chloro-N,N-2-trimethyl-1-propenylamine (0.036 mL, 0.275 mmol) was added drop wise before stirring for 1 h whilst reaching room temperature. Ammonia gas was bubbled through the reaction mixture for 10 min. The reaction was stirred at room temperature for 3 h. The reaction mixture was diluted with DCM and the organic phase washed with water, dried over MgSO₄ and the solvent removed in vacuo. The residue was purified by flash chromatography eluting with dichloromethane:methanol (96:4). Yield: 90 mg (90%). Mass spectrum (ES-MS (+ve) 400 [M+H]⁺, Retention time 0.98 min. ¹H-NMR (CDCl₃, 400 MHz): δ 6.35 and 5.35 (2×1H, br, NH₂), 5.98 (1H, s, C₄—H), 3.25 (1H, d, C₁₁—H), 2.95 (2H, m, alkyl-H), 2.70-1.75 (15H, m, alkyl —H), 1.55 (3H, s, Me), 1.50 (2H, m, alkyl —H), 1.0 (3H, s, Me).

### Example 49

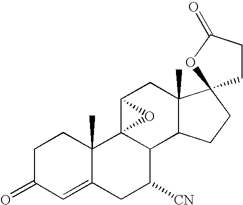

Intermediate 49-1 (46 mg, 0.115 mmol was dissolved in anhydrous THF (8 mL), trifluroacetic anhydride (0.024 mL, 0.172 mmol) was added and the reaction stirred at room temperature for 1.5 h. 5 drops of NaHCO₃ (saturated) was added and solvent removed. The reaction mixture was diluted with DCM and the organic phase washed with saturated NaHCO₃, water, dried over MgSO₄ and the solvent removed in vacuo. The residue was purified by flash chromatography eluting with ethyl acetate:heptane (3:1). Yield: 24 mg (57%). Mass spectrum (ES-MS (+ve) 382 [M+H]⁺, Retention time 1.70 min. ¹H-NMR (CDCl₃, 400 MHz): δ 5.99 (1H, s, C₄—H), 3.20 (1H, d, C₁₁—H), 3.13 (1H, m, alkyl-H), 2.90-1.70 (16H, m, alkyl —H), 1.48 (3H, s, Me), 1.45 (2H, m, alkyl —H), 1.0 (3H, s, Me).

### Example 50

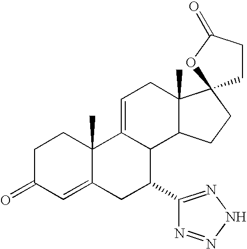

The compound intermediate 6-1 (50 mg, 0.136 mmol) was added to a solution of sodium azide (44 mg, 0.683 mmol) and zinc bromide (153 mg, 0.683 mmol) in DMF (3 mL), and heated at 130° C. for 48 h. The reaction mixture cooled, water and DCM were added, the organic phase was washed with water, dried over MgSO₄ and the solvent removed in vacuo. The residue was purified by flash chromatography eluting with dichloromethane:methanol (9:1). Yield: 14 mg (25%). Mass spectrum (ES-MS (+ve) 409 [M+H]⁺, Retention time 1.10 min. ¹H-NMR (CDCl₃, 400 MHz): δ 5.88 (1H, m, C₁₁—H), 5.98 (1H, s, C₄—H), 4.05(1H, br, NH), 3.10 (1H, m, alkyl-H), 2.85-1.65 (18H, m, alkyl —H), 1.55 (3H, s, Me), 0.95 (3H, s, Me).

### Example 51

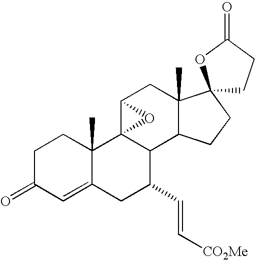

The compound of Example 6 (Aldehyde E; 50 mg, 130 μmol) was dissolved in THF (10 mL). Methoxycarbonylmethylene-triphenylphosphorane (65.3 mg 195 μmol) was added. The reaction mixture was heated under reflux for 24 h. The solvent removed in vacuo. The residue was purified by flash chromatography eluting with ethyl acetate:heptane (3:1). Yield: 24 mg (42%). Mass spectrum (ES-MS (+ve) 441 [M+H]⁺, Retention time 1.37 min. ¹H-NMR (CDCl₃, 400 MHz): δ 6.85(1H, dd, alkene-H), 5.80 (1H, s, C₄—H), 5.75(1H, d, 16 Hz, alkene-H), 3.65(3H, s, OMe), 3.05 (1H, d, C₁₁—H), 2.80 (1H, m, alkyl-H), 2.70-1.45 (16H, m, alkyl-H), 1.44 (3H, s, Me), 1.34 (2H, m, alkyl-H), 0.95 (3H, s, Me).

### Example 52

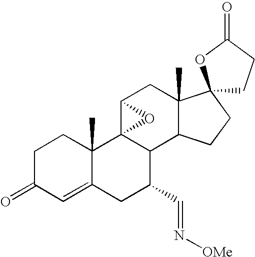

The compound of Example 6 (Aldehyde E; 50 mg, 130 μmol) was dissolved in anhydrous DCM (10 mL). Methoxyamine hydrochloride (11.9 mg, 140 μmol) and potassium carbonate (26.9 mg, 190 μmol) were added and the mixture stirred at room temperature for 3 h. The reaction mixture was diluted with DCM and the organic phase washed with water, dried over MgSO₄ and the solvent removed in vacuo. The residue was purified by flash chromatography eluting with ethyl acetate:heptane (4:1). Yield: 38 mg (66%). Mass spectrum (ES-MS (+ve)) 414 [M+H]⁺, Retention time 1.39 min. ¹H-NMR (CDCl₃, 400 MHz): δ 7.42 (1H, d, oxime CH), 5.87 (1H, s, C₄—H), 3.80 (3H, s, OMe), 3.14 (1H, d, C₁₁—H), 2.90 (2H, m, alkyl-H), 2.65-1.50 (15H, m), 1.50 (3H, s, Me), 1.45 (2H, m, alkyl-H), 1.02 (3H, s, Me).

### Example 53

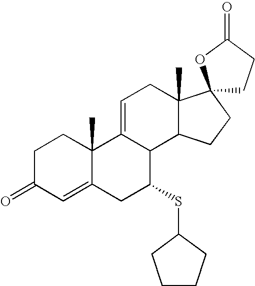

The compound of Example 2 (300 mg, 0.9 mmol) was dissolved in cyclopentanethiol (2 mL) under nitrogen. The reaction mixture was heated to 30° C. Sodium metal (21 mg, 0.9 mmol) was added in one portion and the reaction was re-flushed with nitrogen. The reaction mixture was heated to 60° C. for 3 h and allowed to cool to ambient temperature. Saturated aqueous ammonium chloride was added until pH=7 and the aqueous phase extracted with dichloromethane. The organics were washed with saturated brine solution, dried over MgSO₄ and the solvent evaporated in vacuo. The residue was purified by silica chromatography using 1:2 ethyl acetate:heptane as eluent. Further purification by preparative chromatography gave the required product. Yield: 75 mg (19%). Mass spectrum (ES-MS (+ve)) 441 [M+H]⁺, retention time 1.62 min. ¹H-NMR (CDCl₃, 400 MHz): δ5.8 (1H, s, C₄—H), 5.7 (1H, d, C₁—H), 3.2 (1H, m, CH), 3.05-2.85 (2H, m, CH), 2.75-1.35 (25H, m, CH), 1.35 (3H, s, CH₃), 0.95 (3H, s, CH3).

### Example 54

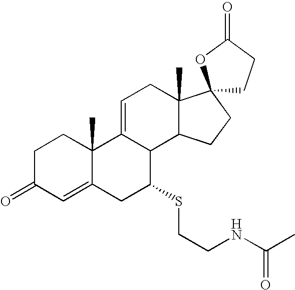

The compound of Example 2 (200 mg, 0.6 mmol) was dissolved in N-acetylcysteamine (2 mL) under nitrogen. The reaction mixture was heated to 30° C. Sodium metal (14 mg, 0.6 mmol) was added in one portion and the reaction was re-flushed with nitrogen. The reaction mixture was then heated to 60° C. for 2 h, allowed to cool and stirred at ambient temperature for 15 h. Saturated aqueous ammonium chloride was added until pH=7 and the aqueous phase extracted with dichloromethane. The organics were washed with saturated brine solution, dried, MgSO₄ and the solvent evaporated in vacuo. The residue was purified by preparative HPLC to give the required product. Yield: 58 mg (21%). Mass spectrum (ES-MS (+ve)) 458 [M+H]⁺, retention time 1.14 min. ¹H-NMR (CDCl₃, 400 MHz):δ 6.1 (1H, s, NH), 5.8 (1H, s, C₄—H), 5.7 (1H, d, C₁₁H), 3.45-3.25 (3H, m, CH), 2.95 (1H, m, CH), 2.7-1.4 (22H, m, CH), 1.4 (3H, s, CH₃), 0.95 (3H, s, CH₃).

### Example 55

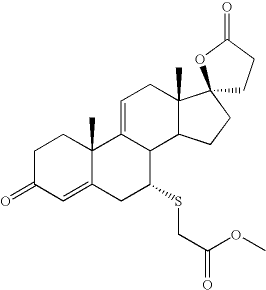

The compound of Example 2 (200 mg, 0.6 mmol) was dissolved in methyl thioglycolate (1 mL) under nitrogen. The reaction mixture was heated to 30° C. Sodium metal (14 mg, 0.6 mmol) was added in one portion and the reaction was re-flushed with nitrogen. The reaction mixture was heated to 60° C. for 2 h, allowed to cool and stirred at ambient temperature for 15 h. Methyl thioglycolate (1 mL) was added and the reaction mixture heated at 60° C. for a further 2 h. Saturated aqueous ammonium chloride was added until pH=7 and the aqueous phase extracted with dichloromethane. The organics were washed with saturated brine solution, dried, MgSO₄ and the solvent evaporated in vacuo. The residue was purified by silica chromatography using 1:2 ethyl acetate:heptane as eluent. Further purification by preparative chromatography gave the required product. Yield: 16 mg (6%). Mass spectrum (ES-MS (+ve)) 445 [M+H]⁺, retention time 1.36 min. ¹H-NMR (CDCl₃, 400 MHz): δ5.8 (1H, s, C₄—H), 5.7 (1H, d, C₁₁—H), 3.7(3H, s, OCH₃), 3.4(1H, m, CH), 3.2(2H, dd, CH₂), 2.9-1.8(18H, m, CH), 1.4(3H, s, CH₃), 0.95(3H, s, CH₃).

### Example 56

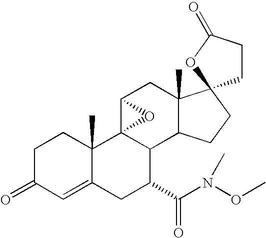

The compound of Example 3 (100 mg, 0.025 mmol) was dissolved in DCM (dichloromethane, 10 mL) and cooled 0° C., 1-chloro-N,N-2-trimethyl-1-propenylamine (0.036 mL, 0.275 mmol) was added drop wise before stirring for 1 h whilst reaching room temperature. In another flask N-methyl O-methyl hydroxylamine hydrochloride (0.026 g, 0.27 mmol) and triethylamine (0.076 mL, 0.054 mmol) were diluted with DCM (5 mL) and the mixture added drop wise to the acid chloride at 0° C. The reaction was allowed to warm to room temperature and stirred at room temperature for 16 h. The reaction mixture was diluted with DCM and the organic phase washed with 0.5M HCl, water, dried over MgSO₄ and the solvent removed in vacuo. The residue was purified by flash chromatography eluting with dichloromethane:methanol (97:3). Yield: 45 mg (41%). Mass spectrum (ES-MS (+ve) 444 [M+H]⁺, Retention time 1.09 min. ¹H-NMR (CDCl₃, 400 MHz): δ 5.75 (1H, s, C₄—H), 3.40 (1H, m, NCOCH), 3.70 and 3.20 (2×3H, 2×S, NMe and OMe), 3.05 (1H, d, C₁₁—H), 2.80 (1H, m, alkyl-H), 2.65-1.50 (16H, m, alkyl —H), 1.48 (3H, s, Me), 1.40 (1H, m, alkyl —H), 1.0 (3H, s, Me).

### Example 57

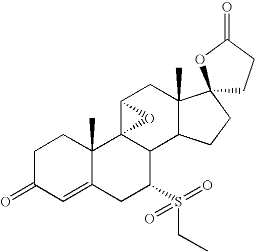

The compound of Example 7 (153 mg, 0.37 mmol) was dissolved in dichloromethane (6 mL). 3-Chloroperoxybenzoic acid (443 mg, 1.92 mmol, 75% purity) was added to the reaction and stirred at ambient temperature for 1.5 h. The reaction mixture was diluted with dichloromethane (10 mL) and washed with 10% aqueous potassium iodide (3×15 mL); saturated aqueous sodium thiosulfate (2×15 mL) and saturated aqueous sodium hydrogen carbonate (15 mL). The organic layers were dried over MgSO₄ and the solvent evaporated in vacuo. The residue was purified by preparative chromatography and then silica chromatography using 3:1 heptane:ethyl acetate to 1:1 heptane:ethyl acetate as eluent to give the desired product. Yield: 24 mg (14%). Mass spectrum (ES-MS (+ve)) 449 [M+H]⁺; 490 [M+H]⁺+MeCN, Retention time 1.28 min. ¹H-NMR (CDCl₃, 400 MHz):δ5.9 (1H, s, C₄—H), 3.4 (2H, m, CH), 3.2 (1H, d, C₁₁—H), 3.1 (2H, m, CH), 2.85-1.7 (15H, m, CH), 1.55 (3H, s, CH₃), 1.45 (2H, m, CH) 1.35 (3H, t, CH₃), 1.05 (3H, s, CH₃).

### Example 58

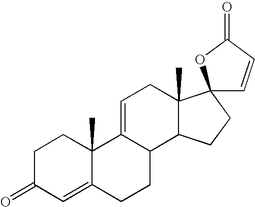

### Intermediate 58-1

### 3-Methylenolether, 5,6,9,11 diene-17 spirolactone intermediate

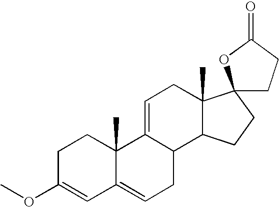

The title compound was prepared from the ester of Example 5 by treatment with NaCl in DMF at reflux as described in WO 97/21720.

### Intermediate 58-2

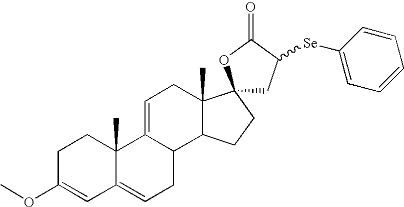

To intermediate 58-1 (1.014 g, 2.86 mmol) was added dry THF (20 mL) under nitrogen. The solution was cooled to −78° C. LiHMDS (1 M solution, 1.2 eq, 3.43 mmol, 3.4 mL) was added dropwise followed by diphenyldiselenide (5 eq, 14.3 mmol, 4.46 g). The resultant mixture was stirred at −78° C. for 5 h, before addition of aqueous saturated ammonium chloride (6 mL), followed by 10% aqueous sodium hydrosulfite (10 mL). The mixture was extracted with ethyl acetate (2×20 mL). The combined organic phases were washed 10% aqueous sodium hydrogen carbonate (20 mL), water (20 mL) and saturated brine (20 mL). The organic phase was further dried over sodium sulfate and evaporated to give an orange solid. Purification of the required selenide was achieved by chromatography on florisil, eluting with heptane:tert-butylmethyl ether (9:1 to 1:1) to give the title compound as a colourless solid (3:2 mixture of diastereomers). Yield: 585 mg, (40%). Mass spectrum (ES-MS (+ve)) 511 [M+H]⁺, Retention time 2.10 min 100% UV. ¹H-NMR (DMSO, 400 MHz): δ 7.67 (2H, m, Ph), 7.43 (3H, m, Ph), 5.54 and 5.45 (1H, 2×dd, C₁₁—H), 5.28 (2H, m, C₄—H, C₆—H), 4.79 and 4.46 (1H, 2×t, C₂₂—H), 3.57 (3H, s, OMe), 2.97 (1H, m, C₈—H), 2.55-1.27 (18H, m), 1.12 and 1.10 (3H, 2×s, Me), 0.89 and 0.86 (3H, 2×s, Me).

### Intermediate 58-3

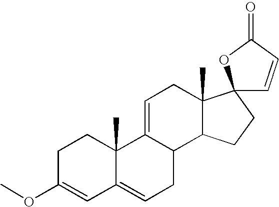

To intermediate 58-2 (150 mg, 0.29 mmol), was added THF (3 mL), MeOH (2 mL) and water (1 mL). Sodium hydrogencarbonate (1.1 eq, 27 mg, 0.32 mmol) was added and the mixture cooled on ice. Hydrogen peroxide (30% aqueous solution, 10 eq, 2.9 mmol, 0.30 mL) was added and the mixture stirred at 0-10° C. for 4 h. After this time, a solution of 10% aqueous sodium sulfite and sodium hydrogen carbonate (20 mL) was added and the reaction allowed to stand. The colourless precipitate was collected by filtration. Yield: 89 mg, (86%). Mass spectrum (ES-MS (+ve)) 353 [M+H]⁺, Retention time 1.85 min, 95% UV. ¹H-NMR (CDCl₃, 400 MHz): δ 7.43 (1H, d, C₂₃—H), 5.97 (1H, d, C₂₂—H), 5.45 (1H, d, C₁₁—H), 5.28 (1H, dd, C₆—H), 5.17 (1H, s, C₄—H), 3.58 (3H, s, OMe), 2.62-1.52 (14H, m), 2.55-1.27 (18H, m), 1.13 (3H, s, Me), 1.06 (3H, s, Me).

### Example 58

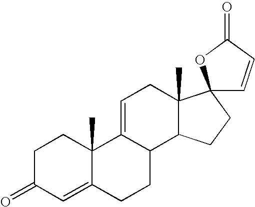

To intermediate 58-3 (13 mg, 37 μmol) was added MeOH—AcOH-water (1:1:1, 1.5 mL). The mixture was stirred at 60° C. for 2.5 h. The mixture was evaporated and the residue purified by chromatography on silica eluting with ethyl acetate to give the required enone. Yield: 9 mg, (72%). Mass spectrum (ES-MS (+ve)) 339 [M+H]⁺, Retention time 1.47 min, 100% UV. ¹H-NMR (CDCl₃, 400 MHz): δ 7.42 (1H, d, C₂₃—H), 5.98 (1H, d, C₂₂—H), 5.77 (1H, s, C₄—H), 5.47 (1H, dd, C₁—H), 2.68-1.65 (10H, m), 1.37 (3H, s, Me), 1.35-1.05 (3H, m), 1.04 (3H, s, Me), 0.87 (1H, m).

### Example 59

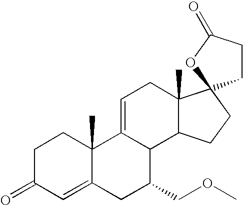

The compound of Example 36 (35 mg, 0.094 mmol) was dissolved in anhydrous dichloromethane (1 mL). Triethylamine (1.1 eq, 0.104 mmol, 15 μl) was added and the mixture cooled to −10° C. Trifluoromethylsulfonic anhydride (1.1 eq, 0.104 mmol, 17.1 μl) was added dropwise and the mixture stirred at −10° C. for 1 h. Methanol (2 mL) was added and the mixture stood at room temperature for 2 days before being evaporated. The residue was purified by chromatography on silica eluting with heptane-ethyl acetate (1:1 to 1:4) to give the methyl ether as a colourless gum (6:1 ratio of α/β diastereomers. Yield: 11 mg, (30%). Mass spectrum (ES-MS (+ve)) 385 [M+H]⁺, Retention time 1.48 and 1.52 min, 91 and 7% UV respectively. ¹H-NMR (CDCl₃, 400 MHz): δ 5.73 and 5.68 (1H, 2×s, C₄H), 5.56 (1H, d, C₁₁—H), 3.16 (3H, s, OMe), 3.14 (1H, m, CH₂OMe), 2.82 (1H, m, CH₂OMe), 2.61-1.38 (19H, m), 1.34 (3H, s, Me), 0.88 (3H, s, Me).

### Example 60

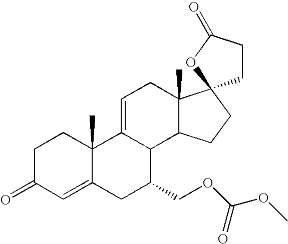

The compound of Example 36 (0.1 g, 0.27 mmol) was dissolved in anhydrous DCM (1 mL). Pyridine (1.1 eq, 0.30 mmol, 24 μl) was added followed by drop wise addition of methyl chloroformate (1.1 eq, 0.30 mmol, 23 μl). The reaction was stirred for 0.5 h at room temperature, evaporated and the crude residue purified by chromatography on silica eluting with heptane-ethyl acetate 3:1 to 1: 1 to give 86 mg of a colourless gum. The material was further purified by HPLC to give the required compound as a colourless solid (3:1 ratio of α:β diastereomers. Yield: 31 mg, (27%). Mass spectrum (ES-MS (+ve)) 429 [M+H]⁺, Retention time 1.49 min, 100% UV. ¹H-NMR (CDCl₃, 400 MHz): δ 5.84 and 5.80 (1H, 2×s, C₄—H), 5.65 (1H, d, C₁₁—H), 4.39-4.06 (1H, m, CH₂OCO.OMe), 3.83 and 3.77 (3H, 2×s, OMe), 3.72 (1H, m, CH₂OCO.OMe), 2.74-1.45 (19H, m), 1.39 (3H, 2×s, Me), 0.97 and 0.93 (3H, 2×s, Me).

### Example 61

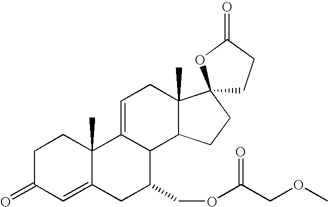

The compound of Example 36 (0.1 g, 0.27 mmol) was dissolved in anhydrous dichloromethane (1 mL). Pyridine (1.1 eq, 0.30 mmol, 24 μl) was added followed by the drop wise addition of 2-methoxyacetyl chloride (1.1 eq, 0.03 mmol, 27 μl). The reaction mixture was stirred for 0.5 h at room temperature, evaporated and the crude residue purified by chromatography on silica eluting with heptane:ethyl acetate 3:1 to 1: 1 to give 60 mg of a colourless gum. The material was further purified by HPLC to give the required compound as a colourless solid (6:1 ratio of α:β diastereomers. Yield: 25 mg, (20%). Mass spectrum (ES-MS (+ve)) 443 [M+H]⁺, Retention time 1.40 min 100% UV. ¹H-NMR (CDCl₃, 400 MHz): δ 5.86 (1H, s, C₄—H), 5.67 (1H, d, C₁₁—H), 4.16 (1H, m, CH₂OCO), 4.01 (2H, s, CO.CH₂OMe), 3.57 (1H, m, CH₂OCO), 3.45 (3H, s, OMe), 2.74-1.43 (19H, m), 1.42 (3H, s, Me), 0.96 (3H, s, Me).

### Example 62

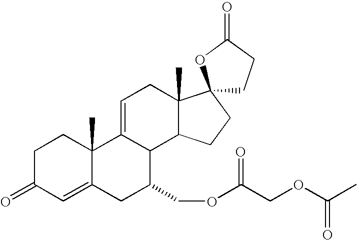

The compound of Example 36 (0.1 g, 0.27 mmol) was dissolved in anhydrous dichloromethane (1 mL). Pyridine (1.1 eq, 0.30 mmol, 24 μl) was added followed by the dropwise addition of 2-acetoxyacetyl chloride (1.1 eq, 0.03 mmol, 32 μl). The reaction mixture was stirred for 0.5 h at room temperature, evaporated and the crude residue purified by chromatography on silica eluting with heptane:ethyl acetate 3:1 to 1: 1 to give 97 mg of a colourless gum. The material was further purified by HPLC to give the required compound as a colourless solid (6:1 ratio of α:β diastereomers. Yield: 39 mg, (33%). Mass spectrum (ES-MS (+ve)) 471 [M+H]⁺, Retention time 1.47 min, 100% UV. ¹H-NMR (CDCl₃, 400 MHz): δ 5.87 (1H, s, C₄—H), 5.67 (1H, d, C₁₁—H), 4.56 (2H, s, CH₂OAc), 4.19 (1H, m, CH₂OCO), 3.79 (1H, m, CH₂OCO), 2.77-2.19 (13H, m), 2.17 (3H, s, OAc), 2.16-1.43 (6H, m), 1.41 (3H, s, Me), 0.94 (3H, s, Me).

### Example 63

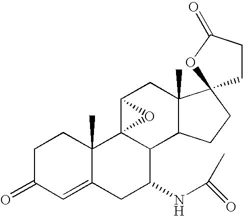

The intermediate 22-1 (100 mg, 0.269 mmol) was dissolved in DCM (10 mL) and triethyl amine (0.04 g, 0.4 mmol) added. To this mixture acetyl chloride (0.025 g, 0.32 mmol) was added drop wise. The reaction mixture was stirred at room temperature for 16 h. The reaction mixture was diluted with DCM, the organic layer washed with saturated NaHCO₃, dried over MgSO₄ and the solvent removed in vacuo. The residue was purified by flash column chromatography eluting with dichloromethane:methanol (97:3). Yield: 85 mg (77%). Mass spectrum (ES-MS (+ve) 414 [M+H]⁺, Retention time 1.18 min. ¹H-NMR (CDCl₃, 400 MHz): δ 6.30(1H, d, NH), 5.85 (1H, s, C₄—H), 4.45 (1H, m, C₇—H), 3.20 (1H, d, C₁₁—H), 2.80-1.65 (17H, m, alkyl-H ), 2.05 (3H, s, COMe), 1.45 (3H, s, Me), 1.40 (1H, m, alkyl-H), 1.05 (3H, s, Me).

### Example 64

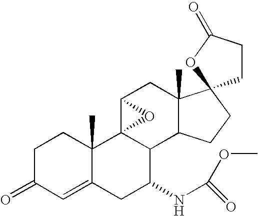

The isocyanate described in the preparation of 22-1 (100 mg, 0.25 mmol) was dissolved in methanol (10 mL) and heated at 50° C. for 16 h. The solvent was removed in vacuo. The residue was purified by flash column chromatography eluting with ethyl acetate:heptane (4:1). Yield: 85 mg (85%). Mass spectrum (ES-MS (+ve) 430 [M+H]⁺, Retention time 1.16 min. ¹H-NMR (CDCl₃, 400 MHz): δ 5.85 (1H, s, C₄—H), 5.4(1H, d, NH), 4.15 (1H, m, C₇—H), 3.66(3H, s, OMe), 3.15 (1H, d, C₁₁—H), 2.70-1.65 (17H, m, alkyl-H ), 1.49 (3H, s, Me), 1.45 (1H, m, alkyl-H), 1.02 (3H, s, Me).

### Example 65

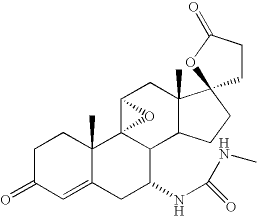

The isocyanate described in the preparation of 22-1 (120 mg, 0.30 mmol) was dissolved in DCM (10 mL) methyl amine hydrochloride (22 mg, 0.33 mmol) was added. To this mixture triethyl amine (0.036 g, 0.36 mmol) was added. The reaction mixture was stirred at room temperature for 16 h. The reaction mixture was diluted with DCM, the organic layer washed with water, dried over MgSO4 and the solvent removed in vacuo. The residue was purified by flash column chromatography eluting with Ethyl acetate:Methanol (97:3). Yield: 77 mg (76%). Mass spectrum (ES-MS (+ve) 429 [M+H]⁺, Retention time 1.02 min. ¹H-NMR (CDCl₃, 400 MHz): δ 5.85 (1H, s, C₄—H), 5.00 (1H, d, NH), 4.45(1H, br, NH), 4.35 (1H, m, C₇—H), 3.15 (1H, d, C₁₁—H), 2.74(3H, s, NMe), 2.68-1.70 (17H, m, alkyl-H ), 1.49 (3H, s, Me), 1.44 (1H, m, alkyl-H), 1.02 (3H, s, Me).

### Example 66

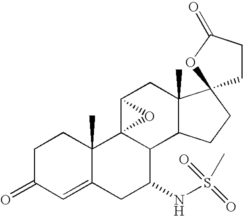

The intermediate 22-1 (75 mg, 0.20 mmol) was dissolved in DCM (8 mL) and triethyl amine (0.03 g, 0.3 mmol) added. To this mixture methanesulfonyl chloride (0.027 g, 0.24 mmol) was added drop wise. The reaction mixture was stirred at room temperature for 16 h. The reaction mixture was diluted with DCM, the organic layer washed with saturated NaHCO₃, dried over MgSO₄ and the solvent removed in vacuo. The residue was purified by flash column chromatography eluting with DCM:Methanol (97:3). Yield: 90 mg (100%). Mass spectrum (ES-MS (+ve) 450 [M+H]⁺, Retention time 1.09 min. ¹H-NMR (CDCl₃, 400 MHz): δ 5.90 (1H, s, C₄—H), 5.10 (1H, d, NH), 4.05 (1H, m, C₇—H), 3.15 (1H, d, C₁₁—H), 2.95 (3H, s, SO₂Me), 2.80-1.65 (17H, m, alkyl-H ), 1.45 (3H, s, Me), 1.40 (1H, m, alkyl-H), 1.05 (3H, s, Me).

### Example 67

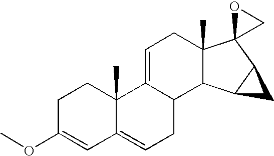 The title compound was used as intermediate.

### Intermediate 67-1

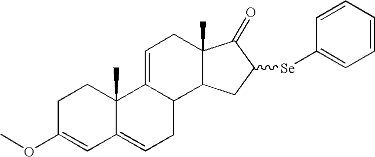

To the appropriate ketone of Formula S6 (15 g, 50.3 mmol) in anhydrous THF (480 mL) under nitrogen was added LiHMDS (1M in THF, 1.15 eq, 57.8 mL, 57.8 mmol). The mixture was stirred at room temperature for 0.5 h and cooled to −78° C. To a separate flask was added diphenyldiselenide (8.81 g, 27.7 mmol) and anhydrous THF (100 mL). Bromine (1.42 mL, 27.7 mmol) was added and the mixture stirred at room temperature for 0.25 h. The phenylselenium bromide solution cooled to −78° C. was added dropwise via cannular to the cold enolate. The mixture was stirred at −78° C. for 1 h before addition of saturated aqueous ammonium chloride (100 mL) at −78° C. The mixture was diluted with ethyl acetate (1000 mL), washed with 1M sodium bicarbonate solution (500 mL), water (500 mL) and saturated brine (500 mL). The organic layer was dried, sodium sulphate and evaporated to give an orange gum (2:1 mixture of diastereomers at C16) which was used in the next step without further purification. Yield: 24.6 g Mass spectrum (ES-MS (+ve)) 454 [M+H]⁺, Retention time 2.90 and 2.94 min, 66:31% UV. ¹H NMR (d₆-DMSO, 400 MHz): [symbol] 7.62 (2H, m, Ph), 7.38 (3H, m, Ph), 5.57 (1H, m, C₁₁—H), 5.25 (2H, m, C₄H, C₆H), 4.47 and 4.09 (1H, 2×dd, C₁₆H), 3.53 (3H, s, OMe), 2.59-0.61 (18H, m).

### Intermediate 67-2

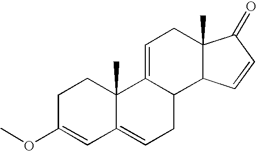

The crude selenide intermediate 67-1 (50.3 mmol) was dissolved in DCM (250 mL) and cooled to −40° C. mCPBA (1.1 eq, 12.7 g, 55.3 mmol) was added in five portions over 0.5 h. The mixture was stirred between −40 and −30° C. for 2 h. Diisopropylamine (3.3 eq, 23.1 mL, 166 mmol) was added. The reaction mixture was transferred via cannular to a solution of DCE/iPr₂NH (9:1, 500 mL) at reflux and the solution heated at reflux for 15 min. The solvent was removed and the residue dissolved in DCM, washed with saturated NaHCO₃, brine and dried (Na₂CO₃) before evaporation to a black gum. The residue was purified by dry flash chromatography on silica (pre-treated with heptane/triethylamine (49:1). The crude material was eluted with heptane/ethyl acetate/triethylamine (89:10:1). The fractions containing the required product were combined and purified by chromatography, silica (pre-treated with heptane/triethylamine (49:1), eluting with heptane/triethylamine/ethyl acetate (99:1:0→89:1:10) to give the required compound as a yellow solid.

Yield: 9.02 g (61%)

Mass spectrum (ES-MS (+ve)) 297 [M+H]⁺, Retention time 2.53 min, 78% UV.

¹H NMR (d₆-DMSO, 400 MHz): [symbol] 7.78 (1H, dd, C₁₅—H), 6.13 (1H, dd, C₁₇—H), 5.60 (1H, dd, C₁₁—H), 5.40 (1H, m, C₆H), 5.24 (1H, m, C₄H), 3.50 (3H, s, OMe), 2.97-0.96 (16H, m).

### Example 67

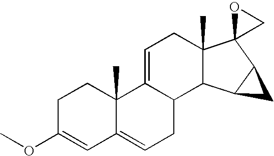

To anhydrous THF and DMSO (1:1 120 mL) was added trimethylsulfoxonium iodide (1.2 eq, 8.21 g, 36.6 mmol). Sodium hydride (1.2 eq, 1.46 g, 36.6 mmol) was added and the mixture stirred at room temperature for 1 h. The intermediate 67-2 enone (9.02 g, 30.5 mmol) was added and the mixture stirred at room temperature for 1 h. To a separate flask was added trimethylsulfonium iodide (3.3 eq, 21 g, 101 mmol), anhydrous DMSO (50 mL) and anhydrous THF (50 mL). Sodium hydride (3.3 eq, 4.03 g, 101 mmol) was added and the resulting slurry stirred at room temperature for 1 h. The trimethylsulfoniun ylide was added rapidly to the mixture, and the mixture stirred at room temperature overnight. The reaction mixture was partially evaporated to remove THF, and diluted with water (200 mL dropwise over 20 min). The resulting precipitate was collected and washed with water (100 mL). The crude brown solid was purified by chromatography on silica which was pre-treated with heptane/triethylamine 49:1). The required product was eluted with heptane/triethylamine/ethyl acetate (99:1:0→94:1:5) as a pale yellow solid.

Yield: 6.06 g (61%)

Mass spectrum (ES-MS (+ve)) 326 [M+H]⁺, Retention time 2.30 min, 72% UV.

¹H NMR (d₆-DMSO, 400 MHz) δ5.36 (1H, dd, C₁₁—H), 5.28 (1H, dd, C₆H), 5.22 (1H, s, C₄H), 3.51 (3H, s, OMe), 2.87 (2H, m, CH₂O), 2.70-1.11 (13H, m), 1.07 (3H, s, Me), 0.82 (3H, s, Me), 0.49 (1H, m).

### Example 68

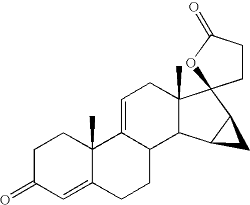 The title compound was used as intermediate

### Intermediate 68-1

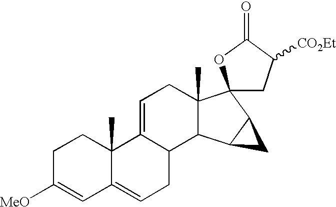

To ethanol (anhydrous, 18.5 mL) under nitrogen, was added diethylmalonate (3 eq, 4.22 mL, 27.8 mmol). Sodium ethoxide of (21% w/w in ethanol, 2.5 eq, 8.64 mL, 23.15 mmol) was added, followed by epoxide Example 67 (3 g, 9.26 mmol). The mixture was heated at reflux for 4 h and cooled to room temperature. Glacial acetic acid (1.6 mL) was added dropwise, followed by the dropwise addition of water (10 mL). The required lactone precipitated and was collected by filtration. The solid was then washed with water and dried overnight under a stream of nitrogen to give a pale yellow solid (C22 5:3 mixture of diastereomers) used in the next step without further purification.

Yield: 3.45 g (85%)

Mass spectrum (ES-MS (+ve)) 439 [M+H]⁺, Retention time 2.75 min, 76% UV.

¹H NMR (d₆-DMSO, 400 MHz) [symbol] 5.53 (1H, 2×dd, C₁₁—H), 5.35 (1H, dd, C₆H), 5.29 (1H, s, C₄H), 4.45 and 3.80 (1H, 2×dd, C₂₂H), 4.27 (2H, 2×q, OCH₂), 3.59 (3H, s, OMe), 2.95-1.41 (15H, m), 1.32 (3H, 2×t, CH₂Me), 1.18 (3H, 2×s, Me), 1.01 and 0.88 (3H, 2×s, Me), 0.55 (1H, m).

### Intermediate 68-2

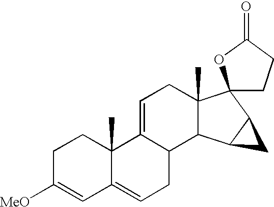

MeO

To anhydrous DMF (2 mL) under nitrogen, was added sodium chloride (2 eq, 234 mg, 4 mmol) and intermediate 68-1 lactone (873 mg, 1.99 mmol). The reaction mixture was heated at 145° C. for 8 h and cooled to room temperature. Water (5 mL) was slowly added and the solidified material broken up. The solid was filtered, washed with water and dried in vacuo at 45° C. to give a pale brown solid. The crude material was contaminated with stage 4 starting material, (15% by LC-MS) which was carried through the next stages.

Yield: 675 mg (93%)

Mass spectrum (ES-MS (+ve)) 367 [M+H]⁺, Retention time 2.61 min, 88% UV.

¹H NMR (d₆-DMSO, 400 MHz δ 5.43 (1H, dd, C₆H), 5.28 (1H, dd, C₆H), 5.22 (1H, s, C₄H), 3.54 (3H, s, OMe), 2.74-0.81 (23H, m), 0.44 (1H, m).

### Example 68

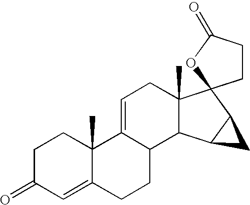

The crude intermediate 68-2 enol ether (747 mg, 1 mmol, containing 15% intermediate 68-1 lactone) was dissolved in acetic acid/methanol/water (5 mL/5 mL/5 mL). The mixture was heated at 65° C. for 45 min and allowed to cool to room temperature. After evaporation, the crude residue was purified by chromatography on silica eluting with heptane/ethyl acetate (3:7) to give the required product as a pale yellow foam.

Yield: 126 mg (36%)

Mass spectrum (ES-MS (+ve)) 353 [M+H]⁺, Retention time 2.19 min, 97% UV.

¹H NMR (CDCl₃, 400 MHz δ5.78 (1H, s, C₄H), 5.48 (1H, dd, C₁₁H), 2.70-2.04 (13H, m), 1.80 (2H, m), 1.54-1.38 (2H, m), 1.30 (3H, s, Me), 1.29-1.12 (2H, m), 0.95 (3H, s, Me), 0.51 (1H, m).

### Example 69

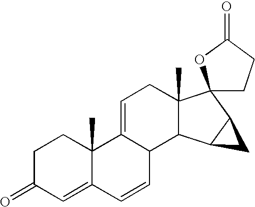

The title compound was used as intermediate

Intermediate 68-2 enol ether (675 mg, 1.84 mmol) was dissolved in DCM (1.4 mL), methanol (0.8 mL) and water (0.24 mL). Chloranil (0.95 eq, 431 mg, 1.75 mmol) was added and the mixture stirred at 42° C. for 2 h, cooled to room temperature and quenched by the addition of 20% w/w aqueous sodium metabisulfite (0.25 mL) and stirred for 0.5 h. Water (5 mL) was added and the mixture stood for 0.5 h. The organic layer was separated and the aqueous re-extracted with DCM (3 mL). The combined organic layers were dried over anhydrous sodium sulphate and evaporated. The residue was re-dissolved in DCM (anhydrous, 15 mL) and stood over powdered anhydrous KOH (310 mg) for 1 h, filtered and evaporated. Purification of the residue on silica eluting with heptane/ethyl acetate (4:1→1:1) gave a pale yellow foam.

Yield: 443 mg (69%)

Mass spectrum (ES-MS (+ve)) 351 [M+H]⁺, Retention time 2.11 min, 90% UV.

¹H NMR (CDCl₃, 400 MHz δ 6.32 (2H, m, C₆H and C₇H), 5.73 (1H, s, C₄H), 5.48 (1H, dd, C₁₁—H), 3.11 (H, m, C₈H), 2.72-1.35 (14H, m), 1.33 (3H, s, Me), 1.02 (3H, s, Me), 0.59 (1H, m).

### Example 70

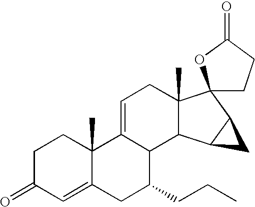

Triene Example 69 (219 mg, 626 μmol) was dissolved in anhydrous THF (5 mL) and ether (5 mL) under nitrogen. Anhydrous LiCl (0.4 eq, 10.6 mg, 250 μmol) and CuCl (0.3 eq, 18.6 mg, 188 μmol) were added. The mixture was cooled to −30° C. nPrMgCl (2M in ether, 1.45 eq, 0.45 mL, 908 μmol) was added dropwise over 20 min. The reaction mixture was stirred at −30° C. for 0.5 h and quenched at this temperature by the addition of 3N HCl (1 mL). The reaction mixture was diluted with ethyl acetate (10 mL), washed with 2N ammonia (2×10 mL), water (10 mL), saturated brine (10 mL) and dried over anhydrous sodium sulphate. Evaporation gave a yellow gum which was purified by chromatography on silica eluting with heptane/ethyl acetate (7:3→1:1) to give the required compound as a colourless gum (7 alpha diastereomer).

Yield: 54 mg (22%)

Mass spectrum (ES-MS (+ve)) 395 [M+H]⁺, Retention time 2.43 min, 100% UV.

¹H NMR (CDCl₃, 400 MHz δ 5.80 (1H, s, C₄H), 5.54 (1H, dd, C₁₁H), 2.69-1.05 (25H, m), 0.95 (3H, s, Me), 0.87 (3H, t, Me), 0.52 (1H, m).

### Example 71

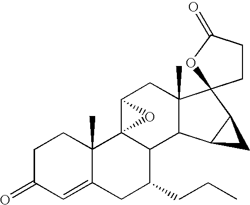

Example 70 (40 mg, 102 μmol) was dissolved in DCM (0.5 mL). mCPBA (1.5 eq, 35 mg, 152 μmol) was added and the mixture stirred overnight at room temperature. Potassium iodide (1 M, 1 mL) and sodium thiosulfate (1 M, 1 mL) were added, followed by sodium bicarbonate (1M, 2 mL). The resulting biphasic mixture was stirred for 0.5 h and the organic layer separated. The aqueous layer was re-extracted with DCM (2 mL) and the combined organic layers dried over anhydrous sodium sulphate. Evaporation gave crude product which was purified by chromatography on silica eluting with heptane/ethyl acetate (1:1) to give the required epoxide as a colourless film.

Yield: 22 mg (48%)

Mass spectrum (ES-MS (+ve)) 411 [M+H]⁺, Retention time 2.26 min, 100% UV.

¹H NMR (CDCl₃, 400 MHz δ5.87 (1H, s, C₄H), 2.98 (1H, dd, C₁₁H), 2.71-1.09 (24H, m), 1.05 (3H, s, Me), 0.89 (3H, t, Me), 0.85 (1H, m), 0.52 (1H, m).

### Example 72

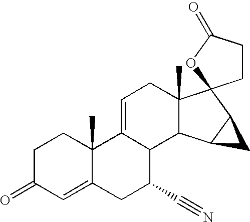 The title compound was used as intermediate

To the triene Example 69 (1.39 g, 3.96 mmol) was added THF (anhydrous, 16 mL) under nitrogen. Diethylaluminum cyanide (1M in toluene, 2.5 eq, 9.91 mL, 9.9 mmol) was added and the mixture heated at reflux for 20 min. The mixture was cooled and poured onto ice (˜50 g). Ice-cold 1M sodium hydroxide (15 mL) and saturated sodium potassium tartrate (60 mL) were added. The mixture was extracted with DCM (2×50 mL). The combined organic layers were washed with ice-cold 1M HCl (50 mL) and dried over anhydrous sodium sulphate. Evaporation gave an orange gum which was purified by chromatography on silica eluting with heptane/ethyl acetate (1:1→3:7). The required compound was isolated as a yellow foam (7 alpha diastereomer).

Yield: 438 mg (29%)

Mass spectrum (ES-MS (+ve)) 378 [M+H]⁺, Retention time 1.91 min, 93% UV.

¹H NMR (CDCl₃, 400 MHz δ 5.82 (1H, s, C₄H), 5.77 (1H, dd, C₁₁H), 3.43 (1H, m, C₇H), 2.92 (1H, m, C₈H), 2.73-2.12 (12H, m), 1.87 (1H, m), 1.52 (2H, m), 1.38 (3H, s, Me), 1.30 (1H, m), 0.99 (3H, s, Me), 0.57 (1H, m).

### Example 73

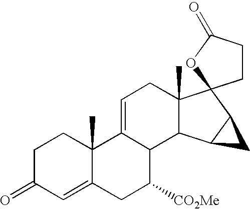 The title compound was used as intermediate

### Intermediate 73-1

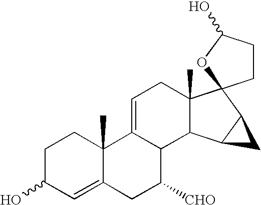

To nitrile Example 72 (312 mg, 828 μmol) was added dry DME (10 mL) under nitrogen and the reaction cooled to 0° C. DIBAL (1M in toluene, 6 eq, 5 mL, 4.98 mmol) was added and the mixture stirred for 2 h at 0-10° C. The reaction was warmed to room temperature and stirred for 4 h. Dibal (1M in toluene, 6 eq, 5 mL, 4.98 mmol) was added and the mixture stirred for 16 h at room temperature. The mixture was cooled on an ice bath and quenched by the slow addition of 3M HCl (5 mL) and diluted with water (10 mL). The organic layer was separated and the aqueous layer extracted with DCM (10 mL). The combined organic layers were dried over anhydrous sodium sulphate and evaporated to give a yellow gum which was used without further purification in the next step.

Yield: 264 mg (83%)

Mass spectrum (ES-MS (+ve)) 367 [M+H−H₂O]⁺, Retention time 1.82 min, 85% UV.

### Intermediate 73-2

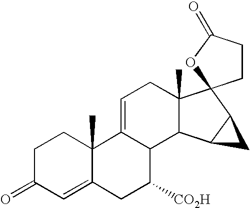

Crude intermediate 73-1 aldehyde (264 mg, 695 μmol) was dissolved in acetone (15 mL) and cooled to 0° C. Jones reagent (8N CrO₃ in sulphuric acid, 1.5 mL) was added and the mixture stirred at 0° C. for 1 h. The reaction was quenched by the addition of ethanol (1 mL). The reaction was diluted with ethyl acetate (15 mL) and the organic layer separated from the chromium salts. The organic layer was extracted with ice-cold saturated sodium bicarbonate solution (3×10 mL) and the combined extracts of the aqueous layer were cautiously neutralised with conc HCl. The aqueous layer was then extracted with DCM (3×10 mL), dried over anhydrous sodium sulphate and evaporated to give the crude acid which was used in the next step without further purification.

Yield: 67 mg (20%)

Mass spectrum (ES-MS (+ve)) 397 [M+H]⁺, Retention time 1.81 min, 72% UV.

### Example 73

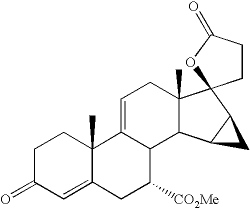

The crude intermediate 73-2 carboxylic acid was dissolved in THF (2 mL) and methanol (1 mL). TNS-Diaomethane (2M in hexane, 2 eq, 170 μl, 340 μmol) was added and the mixture stirred for 1 h at room temperature. The reaction mixture was evaporated and the crude residue purified by chromatography on silica eluting with heptane-ethyl acetate (1:1→3:7) to give the required ester as a colourless solid.

Yield: 41 mg (59%)

Mass spectrum (ES-MS (+ve)) 411 [M+H]⁺, Retention time 2.04 min, 100% UV.

¹H NMR (CDCl₃400 MHz)δ 5.73 (1H, s, C₄H), 5.59 (1H, dd, C₁₁—H), 3.63 (3H, m, OMe), 3.22 (1H, m, C₇H), 2.89 (1H, m C₈H), 2.71-1.30 (19H, m), 0.97 (3H, s, Me), 0.54 (1H, m).

### Example 74

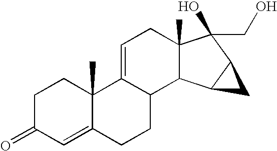 The title compound was used as intermediate

### Intermediate 74-1

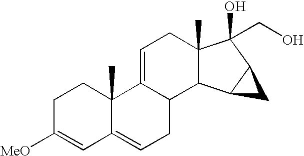

Spiro epoxide Example 67 (2.86 g, 8.81 mmol) was dissolved in DMSO (42 mL) and water (8 mL). Potassium hydroxide (3 eq, 1.48 g, 26.4 mmol) was added and the mixture heated under nitrogen at 115° C. for 1.5 h. The mixture was cooled to room temperature and acidified with glacial acetic acid (1.5 mL), diluted with water (50 mL), neutralised with 1M sodium bicarbonate and extracted with DCM (2×50 mL). The organic layer was partially reduced in volume, diluted with ethyl acetate (50 mL) and washed with water (5×50 mL), saturated brine (50 mL) and dried (anhydrous sodium sulphate). Evaporation gave a dark red solid which was used without further purification.

Yield: 2.64 g (88%)

Mass spectrum (ES-MS (+ve)) 343 [M+H]⁺, Retention time 2.26 min, 65% UV.

¹H NMR (CDCl₃400 MHz δ5.49 (1H, dd, C₁₁H), 5.36 (1H, dd, C₆H), 5.22 (1H, s, C₄H), 3.78 (1H, d, CH₂O), 3.62 (3H, s, OMe), 3.60 (1H, d, CH₂O), 2.75-0.83 (21H, m), 0.43 (1H, m).

*¹H NMR spectrum was obtained by passing CDCl₃ through anhydrous K₂CO₃ before use.

### Example 74

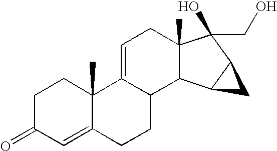

The crude intermediate 74-1 methyl enol ether (230 mg, 673 μmol) was dissolved in THF (6 mL) and 1M HCl (6 mL) and stirred at room temperature for 1 h. After this time the mixture was diluted with ethyl acetate (15 mL) and extracted with 1M sodium bicarbonate (2×10 mL), saturated brine (10 mL) and dried over anhydrous sodium sulphate. Evaporation gave a brown gum which was used without further purification.

Yield: 194 mg (88%)

Mass spectrum (ES-MS (+ve)) 329 [M+H]⁺, Retention time 1.77 min, 71% UV.

### Example 75

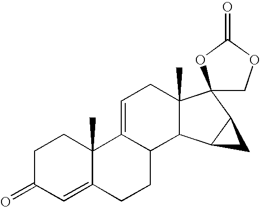

Example 74 diol (133 mg, 405 μmol) was dissolved in dry DCM (4 mL). Dry pyridine (2.4 eq, 79 μl, 972 μmol) was added followed by triphosgene (0.4 eq, 48 mg, 162 μmol) and the resulting mixture stirred at room temperature for 2 h. Ice-cold 1M HCl (5 mL) was added and the organic layer separated. The aqueous was re-extracted with DCM (2 mL) and the combined organic layers dried (anhydrous sodium sulphate). Evaporation gave a brown gum which was further purified by chromatography on silica eluting with heptane-ethyl acetate (2:1→1:1) to give the required carbonate.

Yield: 47 mg (33%)

Mass spectrum (ES-MS (+ve)) 355 [M+H]⁺, Retention time 2.16 min, 94% UV.

¹H NMR (CDCl₃, 400 MHz δ5.76 (1H, s, C₄H), 5.51 (1H, dd, C₁₁H), 4.49 (1H, d, CH₂O), 4.25 (1H, d, CH₂O), 2.71-1.14 (18H, m), 1.00 (3H, s, Me), 0.66 (1H, m).

### Example 76

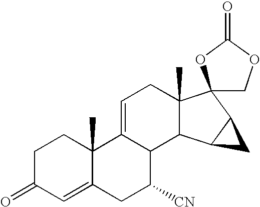

### Intermediate 76-1

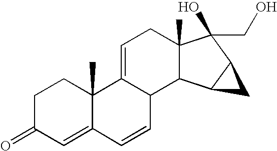

Intermediate 74-1 methyl enol ether (2.64 g, 7.72 mmol) was dissolved in DCM, methanol and water (15 mL/9 mL/2.7 mL). Chloranil (0.93 eq, 1.77 g, 7.2 mmol) was added and the mixture heated at 42° C. for 2 h. The reaction was cooled to room temperature and quenched with 20% w/w sodium metabisulfite (1 mL). The mixture was stirred for 0.5 h, diluted with water (20 mL) and allowed to stand for 0.5 h. The organic layer was separated and the aqueous layer extracted with DCM (10 mL). The combined organic layers were dried over anhydrous sodium sulphate and evaporated to give a dark red gum. Purification by chromatography on silica eluting with heptane, ethyl acetate (1:9) gave the required stage I diol.

Yield: 1.18 g (47%)

Mass spectrum (ES-MS (+ve)) 327 [M+H]⁺, Retention time 1.75 min, 91% UV.

¹H NMR (CDCl₃, 400 MHz δ6.27 (2H, m, C₆H and C₇H), 5.74 (1H, s, C₄H), 5.47 (1H, dd, C₁₁—H), 3.72 (1H, d, CH₂O), 3.47 (1H, d, CH₂O), 3.07 (1H, d, C₈H), 2.74-1.42 (11H, m), 1.30 (3H, s, Me), 1.19 (1H, m), 0.99 (3H, s, Me), 0.49 (1H, m).

### Intermediate 76-2

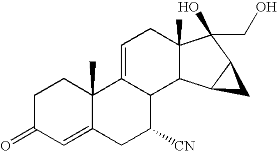

Intermediate 76-1 triene (1.18 g, 3.6 mmol) was dissolved in anhydrous THF (35 mL) under nitrogen. Diethylaluminium cyanide (1M in toluene, 3 eq, 10.9 mL, 10.9 mmol) was added and the mixture heated at 80° C. for 0.75 h. After this time the mixture was cooled to room temperature and poured onto ice (100 g). Saturated sodium potassium tartrate (35 mL) and ice-cold 1M sodium hydroxide (15 mL) were added with stirring. The pH was adjusted to 6.5 by the addition of 1M HCl (20 mL). The mixture was the extracted with DCM (3×15 mL), the organic extracts combined and dried (anhydrous sodium sulphate). After evaporation, the crude residue was purified by chromatography on silica eluting with heptane/ethyl acetate (1→4 to 100%) to give the required nitrile (7 alpha/beta, 4:1 mixture of diastereomers).

Yield: 753 mg (59%)

Mass spectrum (ES-MS (+ve)) 354 [M+H]⁺, Retention time 1.56 min, 98% UV.

¹H NMR (CDCl₃, 400 MHz (7 alpha diastereomer)δ5.90 (1H, s, C₄H), 5.75 (1H, dd, C₁₁—H), 3.78 (1H, d, CH₂O), 3.64 (1H, d, CH₂O), 3.36 (1H, m, C₇H), 2.88 (1H, d, C₈H), 2.70-0.91 (20H, m), 0.49 (1H, m).

### Example 76

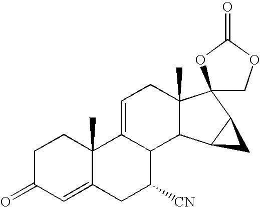

Intermediate 76-2 (56 mg, 159 μmol) was dissolved in anhydrous DCM (2 mL). Anhydrous pyridine (2.4 eq, 31 μl, 382 [μmol) was added followed by triphosgene (0.4 eq, 19 mg, 64 μmol). The mixture was stirred at room temperature for 2 h. Ice-cold 1M HCl (2 mL) was added and the organic layer separated. The aqueous layer was re-extracted with DCM (2 mL) and the combined organic layers dried over anhydrous sodium sulphate. Evaporation gave crude product which was further purified by chromatography on silica eluting with heptane-ethyl acetate (1:1→3:7) to give the required carbonate (7 alpha/beta 7:1 mixture of diastereomers).

Yield: 33 mg (55%)

Mass spectrum (ES-MS (+ve)) 380 [M+H]⁺, Retention time 1.96 min, 98% UV.

¹H NMR (CDCl₃, 400 MHz δ5.91 (1H, s, C₄H), 5.77 (1H, dd, C₁₁—H), 4.56 (1H, d, CH₂O), 4.31 (1H, d, CH₂O), 3.36 (1H, m, C₇H), 2.90 (1H, d, C₈H), 2.68-1.35 (15H, m), 1.08 (3H, s, Me), 0.71 (1H, m).

### Example 77

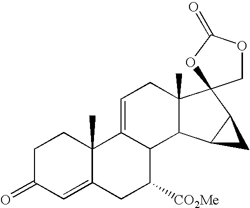

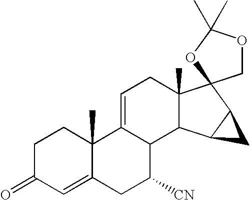

### Intermediate 77-1

To intermediate 76-2 diol (700 mg, 1.98 mmol) was added acetone (100 mL) and p-toluenesulfonic acid (5 mol %, 19 mg, 100 μmol). The mixture was stirred at room temperature for 3 h. The solvent was removed and the residue dissolved in ethyl acetate (20 mL). The organic phase was washed, 1M sodium bicarbonate (20 mL), saturated brine (20 mL) and dried (anhydrous sodium sulphate). Evaporation gave a yellow solid which was used without further purification (7 alpha/beta, 8:1 mixture of diastereomers).

Yield: 749 mg (96%)

Mass spectrum (ES-MS (+ve)) 394 [M+H]⁺, Retention time 2.19 and 2.26 min, 82 and 11% UV respectively.

### Intermediate 77-2

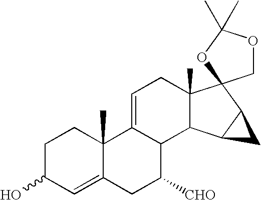

Intermediate 77-1 (749 mg, 1.91 mmol) was dissolved in anhydrous DME (30 mL) under nitrogen. The solution was cooled on ice to 0-10° C. Dibal (1M in toluene, 4 eq, 7.6 mL, 7.6 mmol) was added and the mixture stirred at 0-10° C. for 2 h. The reaction was cautiously quenched with water (20 mL). The pH was adjusted to 5 by addition of 10% v/v acetic acid (6 mL), neutralised with 1M sodium bicarbonate (˜2 mL), diluted with saturated sodium potassium tartrate (20 mL) and extracted with DCM (3×15 mL). The combined extracts were dried over sodium sulphate and evaporated to give a brown foam which was used in the next step without further purification.

Yield: 735 mg (97%)

Mass spectrum (ES-MS (+ve)) 381 [M+H−H₂O]⁺, Retention time 2.22 min, 76% UV.

### Intermediate 77-3

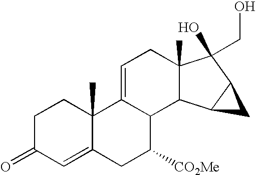

Intermediate 77-2 aldehyde (735 mg, 1.85 mmol) was dissolved in acetone (60 mL) and cooled to 0° C. Jones reagent (8N CrO₃ in sulphuric acid, 1.01 mL) was added and the mixture stirred at 0° C. for 1 h. After this time, the reaction was quenched by the addition of ethanol (1.5 mL). The reaction was diluted with saturated brine (50 mL) and extracted into ethyl acetate (3×15 mL). The combined organic layers were evaporated and re-dissolved in THF (80 mL). 1M HCl (240 mL) was added and the solution stirred at 30° C. for 1 h. The mixture was extracted with DCM (2×100 mL). The combined organic layers were washed with water (100 mL), saturated brine (100 mL) and dried over anhydrous sodium sulphate. Evaporation of the solvent gave a pale yellow gum (600 mg, 87%) which was redissolved in THF (10 mL) and methanol (5 mL). TMS-Diazomethane (2M in hexanes, 2 eq, 1.9 mL, 1.9 mmol) was added and the mixture stirred at room temperature for 0.5 h. Evaporation gave a pale yellow gum which was purified by chromatography on silica eluting with heptane/ethyl acetate (1→4 to 100%) to give the required intermediate 77-3 ester.

Yield: 228 mg (32%)

Mass spectrum (ES-MS (+ve)) 387 [M+H]⁺, Retention time 1.67 min, 86% UV.

### Example 77

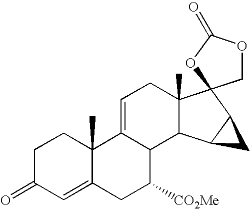

Intermediate 77-3 diol (228 mg, 591 μmol) was dissolved in anhydrous DCM (6 mL). Anhydrous pyridine (2.4 eq, 115 μl, 1.4 mmol) was added followed by triphosgene (0.4 eq, 70 mg, 236 μmol) and the resulting mixture stirred at room temperature for 2 h. Ice-cold 1M HCl (6 mL) was added and the organic layer separated. The aqueous phase was extracted with DCM (6 mL) and the combined organic layers dried over anhydrous sodium sulphate. Evaporation gave a pale yellow gum which was purified by chromatography on silica eluting with heptane/ethyl acetate (1:1 →3:7) to give the required cyclic carbonate (146 mg, 85% UV purity, 60%). The compound of example 77 was obtained following further purification by prep HPLC.

Yield: 88 mg (36%)

Mass spectrum (ES-MS (+ve)) 413 [M+H]⁺, Retention time 2.07 min, 91% UV.

¹H NMR (CDCl₃, 400 MHz δ5.74 (1H, s, C₄H), 5.63 (1H, dd, C₁₁H), 4.49 (1H, d, CH₂O), 4.25 (1H, d, CH₂O), 3.60 (3H, s, Me), 3.20 (1H, m, C₇H), 2.88 (1H, d, C₈H), 2.69-1.46 (12H, m), 1.39 (3H, s, Me), 1.03 (3H, s, Me), 0.67 (1H, m).

### Example 78

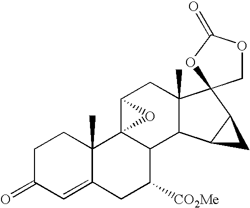

Example 77 (58 mg, 141 μmol) was dissolved in anhydrous DCM (2 mL). mCPBA (1.5 eq, 49 mg, 211 mmol 75% purity) was added and the mixture stirred for 4 h at room temperature. mCPBA (1 eq, 33 mg, 144 μmol) was added and the mixture stirred for 4 h at room temperature. 1M Potassium iodide (1 mL) and 1M sodium thiosulfate (1 mL) were added and the reaction stirred at room temperature for 0.5 h. 1M Sodium bicarbonate (2 mL) was added and the organic layer separated. The aqueous layer was extracted with DCM (2 mL×2) and the combined layers dried (anhydrous sodium sulphate). Evaporation gave a crude product which was purified by chromatography on silica eluting with heptane-ethyl acetate 1:1 to give the required epoxide.

Yield: 22 mg (37%)

Mass spectrum (ES-MS (+ve)) 429 [M+H]⁺, Retention time 1.93 min, 92% UV.

¹H NMR (CDCl₃, 400 MHz) δ5.85 (1H, s, C₄H), 4.45 (1H, d, CH₂O), 4.20 (1H, d, CH₂O), 3.60 (3H, s, Me), 3.05 (2H, m, C₇H, C₁₁,H), 2.70 (2H, m), 2.55-1.25 (11H, m), 1.50 (3H, s, Me), 1.20 (3H, s, Me), 0.60 (1H, m).

### Example 79

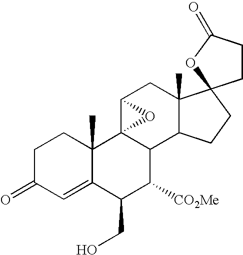

Eplerenone (0.5 g, 1.21 mmol) was added to methanol (3.8 mL) and pyrrolidine (0.21 mL) added. The reaction was heated to reflux for 15 min., cooled to room temperature and filtered. The solid was washed with methanol, air dried and dissolved in ethanol/toluene (11 mL/5 mL). Formaldehyde (37% in water, 164 mL, 2 mmol) was added and the reaction stirred for 30 min at room temperature. The solvent was removed in vacuo to give the required crude material which was purified by column chromatography (ethyl acetate) 207 mg.

Mass spectrum (ES-MS (+ve)) 427 [M−H₂O]⁺ 445 [M+H]⁺, Retention time 1.64 min, 98% UV.

¹H-NMR (CDCl₃, 400 MHz): δ 5.87 (1H, s, C₄—H), 3.80 (1H, m, CH₂OH), 3.68 (1H, m, CH₂OH), 3.60 (3H, s, OMe), 3.06 (1H, d, C₁₁—H), 2.96 (2H, m), 2.60-2.30 (5H, m), 2.25-2.05 (3H, m), 2.0 (1H, m), 1.81 (4H, m), 1.70-1.25 (3H, s, Me) 1.70-1.25 (m), 0.96 (3H, s, Me)

### Example 80

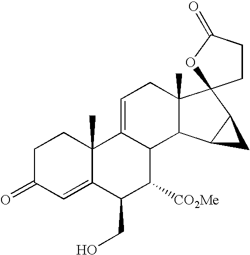

Example 73 (200 mg, 0.48 mmol) was added to methanol (1.5 mL) and pyrrolidine (0.084 mL, 1.02 mmol) added. The reaction was heated to reflux for 15 min., cooled to room temperature and the solid filtered. The solid was washed with MeOH and air dried. The residue was dissolved in ethanol/toluene (4.4 mL/2.2 mL). Formaldehyde (37% in water, 0.174 mL, 2.34 mmol) was added and the reaction stirred for 30 min at room temperature. The solvent was removed in vacuo to give the required crude product which was purified by column chromatography (EtOAc:heptane 4:1).

Mass spectrum (ES-MS (+ve)) 441 [M+H]⁺, Retention time 1.82 min, 92% UV.

¹H-NMR (CDCl₃, 400 MHz): δ 5.76 (1H, s, C₄—H), 5.56 (1H, m, C₁₁—H), 3.95 (1H, m), 3.73 (1H, m), 3.56, (3H, s, CO₂CH₃), 3.29 (1H, m), 2.75 (1H, m), 2.70-2.40 (5H, m), 2.35-2.00 (5H, m), 1.80 (1H, m), 1.70 (2H, m), 1.60 (1H, m), 1.30 (1H, m) 1.28 (3H, s, Me), 1.25 (1H, m), 0.90 (3H, s, Me), 0.50 (1H, m)

### Example 81

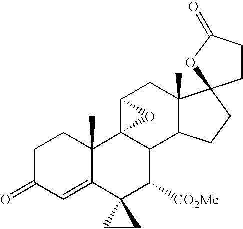

Example 79 (77 mg, 0.17 mmol), was dissolved in pyridine (0.8 mL) under nitrogen and mesyl chloride (39 mL, 0.50 mmol) added. The reaction was stirred at room temperature for 4 h. The reaction mixture was poured onto ice water (10 mL), extracted with DCM (10 mL×3) and the combined organics dried (Na₂SO₄), filtered and concentrated in vacuo to give the mesylate (136 mg) which was used directly in the cyclopropanation step. Me₃S(O)I (116 mg, 0.53 mmol), DMSO (2.8 mL) and NaH (60%, 16 mg, 0.39 mmol) were stirred under nitrogen for 1 h. Crude mesylate was added in DMSO (1 mL) and the reaction stirred for 24 h at room temperature under nitrogen. The reaction mixture was quenched by addition of saturated ammonium chloride (4 mL) at 0° C. and extracted with EtOAc (10 mL) and washed with water (6 mL×3). The organic layer was dried (Na₂SO₄) filtered and the solvent evaporated. The required product was isolated following column chromatography (heptane:ethyl acetate, 3:2).

Mass spectrum (ES-MS (+ve)) 441 [M+H]⁺, Retention time 1.96 min, 100% UV.

¹H-NMR (CDCl₃, 400 MHz): δ 5.67 (1H, s, C₄—H), 3.59 (3H, s, OMe), 3.08 (1H, d, C₁₁—H), 2.67 (1H, dd), 2.60-2.30 (4H, m), 2.30-2.05 (3H, m), 2.0-1.5 (7H, m), 1.48 (3H, s, Me), 1.40-1.25 (3H, m), 0.98 (3H, s, Me), 0.87 (1H, m), 0.57 (2H, m)

### Example 82A & 82B

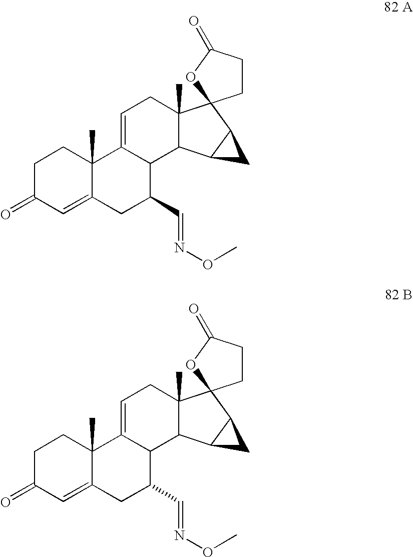

### Intermediate 82-1

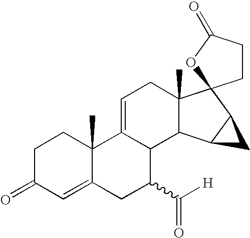

To diol Example 73-1 (284 mg, 0.73 mmol), DCM (8 mL), 4-methyl morpholine-N oxide (259 mg, 2.21 mmol) and activated 4A molecular sieves was added TPAP (25.9 mg, 0.073 mmol) in one portion under nitrogen. The reaction was stirred for 3 h at room temperature. The solvent was removed in vacuo and the crude product purified by chromatography (EtOAc:heptane 3:1) to give the required diketo aldehyde as a 1:1 alpha/beta diastereomeric mixture at C7.

Mass spectrum (ES-MS (+ve)) 381 [M+H]⁺, Retention time 1.89 (46%) & 1.93 min 47% UV.

¹H-NMR (CDCl₃, 400 MHz): 9.50 (1H, m, CHO), 5.96 (1H, 2×s, C₄—H), 5.61 (1H, m, C₁₁—H), 2.87 (2H, m), 2.47-2.44 (6H, m), 2.23-2.10 (5H, m), 2.00-1.40 (2H, m) 1.39 & 1.38 (3H, 2×s, Me), 0.93 & 0.77 (3H, 2×s, Me), 0.89 (1H, m), 0.50 (1H, m)

### Example 82A

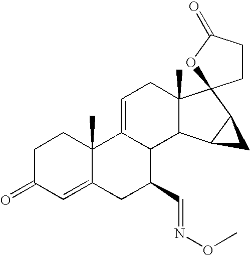 keto aldehyde intermediate 82-1 (78 mg, 0.20 mmol) was dissolved in DCM and O-methyl hydroxylamine hydrochloride (16.2 mg, 0.195 mmol) added. Pyridine (0.023 mL, 0.29 mmol) was added and the reaction stirred at room temperature for 3 h. O-Methyl hydroxylamine hydrochloride (16.2 mg, 0.195 mmol) was added and the reaction stirred for 16 h. The mixture was diluted with DCM, washed with water, dried (MgSO₄) and the crude purified by column chromatography (EtOAc:heptane, 3:2). Two fractions were obtained enriched at C7 with either alpha or beta stereochemistry:

First fraction: 10 mg (11%); 20% 7-alpha; 72% 7-beta

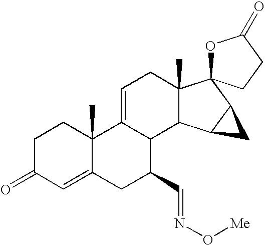

Mass spectrum (ES-MS (+ve)) 410 [M+H]⁺, Retention time 2.12 (72%) & 2.15 min 20% UV.

¹H NMR for major diastereomer:

¹H-NMR (CDCl₃, 400 MHz): δ 6.98 (1H, d, CHN), 5.73 (1H, s, C₄—H), 5.57 (1H, m, C₁₁—H), 3.74 (CH₃, s, OMe), 2.93 (1H, m), 2.88 (1H, m), 2.70-1.95 (12 H, m) 1.73 (1H, m), 1.50 (1H, m), 1.34 (3H, s, Me), 0.90 (3H, s, Me), 0.47 (1H, m).

### Example 82B

Second fraction: 20 mg (18%); 85% 7-alpha, 13% 7-beta

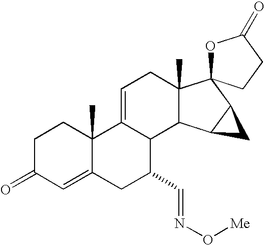

Mass spectrum (ES-MS (+ve)) 410 [M+H]⁺, Retention time 2.12 (13%) & 2.16 min 85% UV.

¹H NMR for major diastereomer

¹H-NMR (CDCl₃, 400 MHz): δ 6.97 (1H, d, CHN), 5.72 (1H, s, C₄—H), 5.59 (1H, m, C₁₁—H), 3.74 (CH₃, s, OMe), 3.00 (1H, m), 2.90 (1H, m), 2.75-2.00 (12H, m) 1.75 (1H, m), 1.57-1.35 (m), 1.33 (3H, s, Me), 0.77 (3H, s, Me), 0.45 (1H, m).

* Note assignments are arbitrary 7 alpha/beta assignment may be the opposite of that indicated

### Example 83

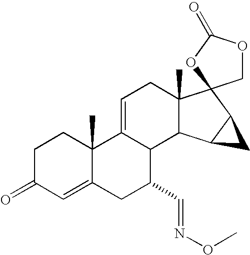

### Intermediate 83-1

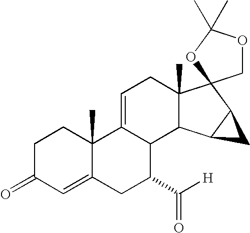

To intermediate 77-2 (600 mg, 1.51 mmol) was added DCM (17 mL) and the mixture cooled to 0° C. 4-Methyl morpholine-N-oxide (176 mg, 1.51 mmol) activated 4A molecular sieves (1.4 g) and TPAP (53 mg, 0.15 mmol) were added under nitrogen. The reaction was stirred for 2 h at room temperature. The reaction was filtered and the solvent removed in vacuo and the crude product. The crude was dissolved in DCM and filtered through a pad of silica eluting with DCM→EtOAc to give the required product.

Mass spectrum (ES-MS (+ve)) 397 [M+H]⁺, Retention time 2.07 min 97% UV.

### Intermediate 83-2

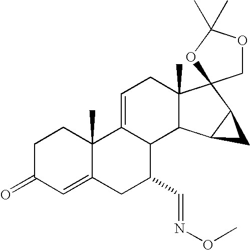

Keto aldehyde intermediate 83-1 (386 mg, 0.97 mmol) was dissolved in DCM (2.5 mL) and O-methyl hydroxylamine hydrochloride (73 mg, 0.87 mmol) added. Pyridine (0.11 mL, 1.36 mmol) was added and the reaction stirred at room temperature for 3 h. The mixture was diluted with DCM, washed with water, dried (MgSO₄) and the crude (413 mg) used in the next step without further purification.

Mass spectrum (ES-MS (+ve)) 426 [M+H]⁺, Retention time 2.23 min 78% UV.

### Intermediate 83-3

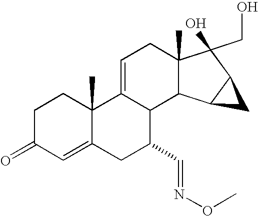

Ketal oxime intermediate 83-2 (413 mg, 0.97 mmol) was dissolved in THF (15 mL) and HCl (1 M aqueous solution, 50 mL) added. The reaction was heated at 30° C. until TLC indicated the reaction was complete. The solvent was removed and the crude (380 mg) used in the next stage without further purification

Mass spectrum (ES-MS (+ve)) 386 [M+H]⁺, Retention time 1.49 min 71% UV.

### Example 83

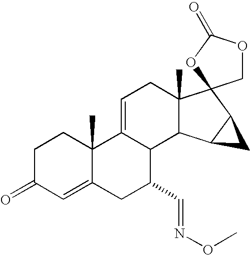

To a solution of intermediate 83-3 diol (0.37 g, 0.96 mmol) in DCM (10 mL) was added triphosgene (0.34 g, 1.15 mmol) and pyridine (0.186 mL, 2.3 mmol). The reaction was stirred at room temperature for 3 h. The reaction mixture was washed with HCl (2 M aqueous) and the organic phase separated and dried (MgSO₄). Compound was obtained following column chromatography (EtOAc:heptane 2:3).

Example 83: 166 mg (42%); 2:1 E/Z mixture

Mass spectrum (ES-MS (+ve)) 411 [M+H]⁺, Retention time 4.19 min (69%) & 4.28 min (28%) UV.

¹ H NMR for E geometric isomer

¹H-NMR (CDCl₃, 400 MHz): δ 6.96 (1H, d, CHN), 5.73 (1H, s, C₄—H), 4.41 & 4.19 (2H, 2×d, C₂₀—CH₂), 5.59 (1H, m, C₁₁—H), 3.74 (CH₃, s, OMe), 3.72 (m), 2.99 (1H, m), 2.90 (1H, m), 2.61 (1H, m), 2.50-2.25 (3H, m) 2.20-1.95 (3H, m), 1.85 (m), 1.69 (1H, m), 1.59 (1H, m), 1.39 (m), 1.34 (3H, s, Me), 0.94 (3H, s, Me), 0.59 (m).

### Example 84

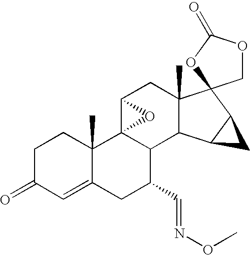

Example 83 (143 mg, 0.34 mmol) was dissolved in DCM (15 mL) and mCPBA (120 mg, 0.69 mmol) added. The reaction was stirred for 2 h and mCPBA (60 mg, 0.34 mmol) added and the reaction stirred at room temperature for a further 48 h The reaction was diluted with DCM (15 mL), quenched by the addition of 10% (weight/volume KI in water), Na₂S₂O₃ (saturated aqueous) and washed with NaHCO₃. The organic layer was separated, dried (Na₂SO₄), filtered and concentrated in vacuo to give the crude product. The crude was purified by column chromatography (heptane:EtOAc, 3:2).

Compound: 40 mg (29%); 9:1 E/Z mixture

Mass spectrum (ES-MS (+ve)) 428 [M+H]⁺, Retention time 4.27 min 90% UV.

¹H NMR for E geometric isomer

¹H-NMR (CDCl₃, 400 MHz): δ 7.37 (1H, d, CHN), 5.83 (1H, s, C₄—H), 4.43 & 4.17 (2H, 2×d, C₂₀—CH₂), 3.75 (CH₃, s, OMe), 3.06 (1H, d), 2.99 (1H, m), 2.90 (1H, m), 2.55-2.25 (4H, m) 2.15 (1H, m), 2.0-1.50 (m), 1.45 (3H, s, Me), 1.40-1.25 (m), 1.04 (3H, s, Me), 0.60 (m).

Notes*: geometric assignment of E as major isomer for Example 83 and example 84 based on previous chemistries.

### Example 85

### In Vitro Competitive Binding Assays

A. Materials and Methods

Mineralocorticoid receptor (MR) binding assay: Spodoptera furgiperda (Sf9) insect cells (ATCC accession number CRL-1711) were maintained in culture in EX-CELL™ 420 serum free medium (JRH Biosciences, Inc.) at 27° C.

A recombinant baculovirus containing nucleotide sequences encoding the ligand binding domain of the human mineralocorticoid receptor (hMR) cloned into the pFastBac™ vector was generated using the Bac-to-Bac® Baculovirus Expression System Kit (Invitrogen) following the manufacturer's instructions. This recombinant baculovirus contains nucleotides 2401 through 3168 (SEQ ID NO: 13) of hMR, providing for expression of the ligand binding domain (LBD), amino acid residues 729-984 (SEQ ID NO: 14), of hMR (hMR LBD). Cultured Sf9 insect cells were transfected with this recombinant baculovirus following the manufacturer's instructions.

For the binding assay, a crude lysate of the hMR LBD-expressing insect cells was prepared. The crude lysate was diluted 12.5-fold with binding buffer (20 mM Tris-HCl pH 7.4, 1 mM EDTA, 1 mM dithiothreitol, 20 mM sodium tungstate, 50 mM KCl and 10% glycerol).

For the negative control sample, the diluted lysate was incubated with a 10 nM final concentration of the radioligand [1,2,6,7-³H] aldosterone (Amersham Biosciences, catalogue # 434) for 3.5 h at 4° C. For test compounds and the positive control sample, the diluted lysate was incubated with various concentrations of test compound or with a 20 μM final concentration of spironolactone (Sigma, S-3378) as a reference compound for 45 min on ice, and was then incubated with a 10 nM final concentration of [1,2,6,7-³H] aldosterone for 3.5 h at 4° C.

Using a CellHarvester (PerkinElmer, formerly Packard Biosciences), receptor/ligand complexes were retained on 96-well GF/B filters (PerkinElmer, formerly Packard Biosciences) while free radioligand passed through the filters. Scintillation counting of GF/B filter plates was performed using a TopCount NXT (PerkinElmer, formerly Packard Biosciences) to quantitate the amount of [1,2,6,7-³H] aldosterone bound to the hMR LBD.

The IC₅₀ of a test compound was determined from the concentration of test compound giving half-maximal displacement as obtained by nonlinear least-squares curve fitting to a one-site binding sigmoidal model using Prism 3.0 (Graph Pad Software, Inc., San Diego, US). The calculated IC₅₀ values represent the average values calculated from the results of 1-3 experiments carried out in duplicate.

Androgen receptor (AR) binding assay: Recombinant ligand binding domain (LBD) of the rat androgen receptor (AR LBD) (PanVera/Invitrogen, catalogue # P2719) was diluted to a final concentration of 5 nM with binding buffer (50 mM Tris-HCl pH 7.5, 2 mM dithiothreitol, 800 mM NaCl, 1 mg/mL BSA and 10% glycerol).

For the negative control reaction, diluted AR LBD was incubated with a 5 nM final concentration of the radioligand [17α-methyl-³H] mibolerone (PerkinElmer, catalogue # NET919) for 4 h at 4° C. For test compounds and the positive control sample, diluted AR LBD was incubated with various concentrations of test compound or a 1 μM final concentration of testosterone as reference compound (Sigma T-1500) and with a 5 nM final concentration of [17α-methyl-³H] mibolerone for 4 h at 4° C. Separation of bound/free ligand and scintillation counting were carried out as described above for the MR binding assay. The IC₅₀ of a test compound was determined as described above.

Estrogen receptor (ER) binding assay: Human recombinant estrogen receptor-α (ER) (PanVera/Invitrogen, catalogue # P2187) was diluted to a final concentration of 4 nM with binding buffer (50 mM Tris-HCl pH 7.4, 0.1 mM EDTA, 2 mM dithiothreitol, 20 mM sodium tungstate, 100 mM KCl, 1 mM CHAPS and 10% glycerol).

For the negative control reaction, diluted ER was incubated with a 5 nM final concentration of the radioligand [6,7-³H] estradiol (Amersham Biosciences, catalogue # TRK125) for 4 h at 4° C. For test compounds and the positive control sample, diluted ER was incubated with various concentrations of test compound or a 2 μM final concentration of 17β-estradiol as reference compound (Calbiochem 3301) a reference compound and a 5 nM final concentration of [6,7-³H] estradiol for 4 h at 4° C. Separation of bound/free ligand and scintillation counting were carried out as described above for the MR binding assay. The IC₅₀ of a test compound was determined as described above.

Glucocorticoid receptor (GR) binding assay: Human recombinant glucocorticoid receptor (GR) (PanVera/Invitrogen, catalogue # P2812) was diluted to a final concentration of 4 nM with binding buffer (10 mM sodium phosphate buffer pH 7.4, 0.1 mM EDTA, 5 mM dithiothreitol, 10 mM sodium molybdate).

For negative control reactions, diluted GR was incubated with a 10 nM final concentration of the radioligand [1,2,4-³H] dexamethasone (Amersham Biosciences, catalogue # TRK417) for 4 h at 4° C. For test compounds and the positive control sample, diluted GR was incubated with various concentrations of test compound or a 2 μM final concentration of dexamethasone (Sigma D-4902) as reference compound and a 10 nM final concentration of the [1,2,4-³H] dexamethasone for 4 h at 4° C. Separation of bound/free ligand and scintillation counting were carried out as described above for the MR binding assay. The IC₅₀ of a test compound was determined as described above.

Progesterone receptor (PR) binding assay: Human recombinant progesterone receptor (PR) (PanVera/Invitrogen, catalogue # P2835) was diluted to a final concentration of 8 nM with binding buffer consisting of a 1: 1 mixture of buffer A (50 mM Tris-HCl pH 8.0, 1 M urea, 20 mM sodium molybdate, 100 mM KCl and 10% glycerol) and buffer B (Red PR screening buffer; PanVera/Invitrogen). This binding buffer was supplemented with 2.5 mM dithiothreitol before use.

For negative control reactions, diluted PR was incubated with 8 nM final concentration of the radioligand [17α-methyl-3H] promegestone (PerkinElmer, catalogue # NET555) for 4 h at 4° C. For test compounds and the positive control sample, diluted PR was incubated with various concentrations of test compound or a 10 μM final concentration of progesterone (Sigma P-8783) a reference compound and an 8 nM final concentration of [17α-methyl-3H] promegestone for 4 h at 4° C.

Separation of bound/free ligand and scintillation counting were carried out as described above for the MR binding assay. The IC₅₀ of a test compound was determined as described above.

IC₅₀ and inhibitory ratio calculations: To provide a measure of the potency of each test compound as an antagonist of MR, the IC₅₀ of each test compound (IC₅₀^{Test}) for ligand binding to MR and the IC₅₀ of the known MR antagonists eplerenone (IC₅₀^{Ep}) and spironolactone (IC₅₀^{Sp}) for ligand binding to MR were used to calculate an IC₅₀ ratio (RIC₅₀) according to the following equations:
RIC₅₀^{Ep/Test}=IC₅₀^{Ep}/IC₅₀^{Test} and
RIC₅₀^{Sp/Test}=IC₅₀^{Sp}/IC₅₀^{Test}.

To provide a measure of the selectivity of test compounds for MR over AR, ER, GR, and PR, the IC₅₀ of each test compound for ligand binding to the AR, ER, GR, or PR receptor and the IC₅₀ of each test compound for ligand binding to MR were used to calculate an inhibitory ratio (IR) according to the following equations:
IR^{AR/MR}=IC₅₀^{AR}/IC₅₀^{MR};
IR^{ER/MR}=IC₅₀^{ER}/IC₅₀^{MR};
IR^{GR/MR}=IC₅₀^{GR}/IC₅₀^{MR}; and
IR^{PR/MR}=IC₅₀^{PR}/IC₅₀^{MR}.

These individual inhibitory ratios were used to calculate a total inhibitory ratio (IR^{TOTAL}) according to the following equation:
IR^{TOTAL}=IR^{AR/MR}+IR^{ER/MR}+IR^{GR/MR}+IR^{PR/MR}.
B. Results and Discussion

The mineralocorticoid receptor (MR) antagonist compounds of the invention were characterized in in vitro competitive binding assays. These assays were used to quantitate the IC₅₀, in nM, of individual antagonist compounds for aldosterone binding to the human MR LBD. These assays were further used to quantitate the IC₅₀, in nM, of the antagonist compounds for mibolerone binding to the androgen receptor (AR), estradiol binding to the human estrogen receptor (ER), dexamethasone binding to the glucocorticoid receptor (GR), and promegestone binding to the progesterone receptor (PR). To provide a measure of the potency of each test compound, IC₅₀ ratios (RIC₅₀) were calculated based upon the IC₅₀ of each test compound for ligand binding to MR, and the IC₅₀ of the known MR antagonists eplerenone and spironolactone for ligand binding to MR. To provide a measure of the selectivity of compounds of formula I for MR versus AR, ER, GR, or PR, the IC₅₀ of each compound for ligand binding to the AR, ER, GR, or PR receptor and the IC₅₀ of each compound for ligand binding to MR were used to calculate inhibitory ratios.

The results of such experiments show that the compounds of formula I are MR antagonists. Some of the compounds of formula I, including Examples 21, 27, 29, 33, 36, 40, 42, 59, 78, 80 and 81 are more potent MR antagonists than eplerenone (RIC₅₀^{Ep/Test}≧1.0), while some of the compounds of formula I, including Examples 7, 11, 12, 13, 18, 23, 26, 28, 34, 44, 58, 70, 71, 75, 76, 77, 82A, 82B, 83 and 84 are more than an order of magnitude more potent than eplerenone (RIC₅₀^{Ep/Test}≧10.0). Similarly, some of the compounds of formula I, including Examples 7 and 12 are comparable to spironolactone in potency (RIC₅₀^{Sp/Test}≧0.5), while some of the compounds of formula I, including Examples 8, 10, 11, 13, 18, 20, 26, 28, 34, 44, 52, 53, 70, 71, 75, 76, 77, 82A, 82B, 83 and 84 are each more potent MR antagonists than spironolactone (RIC₅₀^{Sp/Test}≧1.0).

The results of such experiments further show that the compounds of formula I are selective MR antagonists (i.e., that they are selective for MR over AR, ER, GR, and PR), and further that many of the compounds of formula I are more selective for MR over AR, ER, GR, and PR than the known MR antagonists eplerenone and spironolactone.

Some of the compounds of formula I, including Examples 7, 13 and 52, Example 78, 81 and 84 are at least one order of magnitude more selective for MR vs AR, ER, GR, and PR (i.e. IR^{AR/MR}, IR^{ER/MR}, IR^{GR/MR}, IR^{PR/MR} each ≧10.0), while some of the compounds of formula I, including Examples 46, 70, 75 and 76 are at least two orders of magnitude more selective for MR vs AR, ER, GR, and PR (i.e. IR^{AR/MR}, IR^{ER/MR}, IR^{GR/MR}, and IR^{PR/MR} each ≧100.0).

Some of the compounds of formula I are more selective for MR over AR, ER, GR, and PR than eplerenone. For example, some of the compounds of formula I, including Examples 7, 11, 13, 46, 70, 75 and 76 have an IR^{TOTAL} that is at least an order of magnitude greater than the IR^{TOTAL} for eplerenone.

Further, some of the compounds of formula I are more selective for MR over AR, ER, GR, and PR than spironolactone. For example, some of the compounds of formula I, including Examples 7, 11, 46 and 52 have an IR^{TOTAL} that is greater than the IR^{TOTAL} for spironolactone, while some of the compounds of formula I, including Examples 13, 70, 75 and 76 have an IR^{TOTAL} that is at least an order of magnitude greater than the IR^{TOTAL} for spironolactone.

Particularly preferred compounds of the invention, including Examples 11, 13, 70, 75 and 76 are more potent (e.g., as shown by RIC₅₀^{Ep/Test}≧10.0 and RIC₅₀^{Sp/Test}≧1.0) and more selective (e.g., as shown an IR^{TOTAL} that is both greater than the IR^{TOTAL} for eplerenone and greater than the IR^{TOTAL} for spironolactone) than eplerenone and spironolactone, MR antagonists previously described in the art.

### Example 86

### In Vitro Functional Assays

A. Materials and Methods

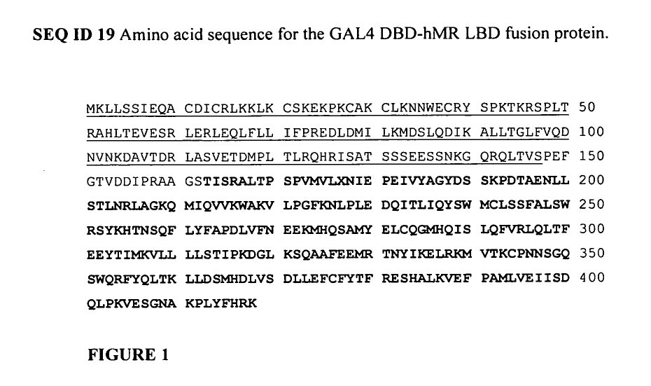

GAL4 DBD-hMR LBD fusion protein expression vector: A pCMX-GAL4-based vector was used for the expression of a fusion protein composed of the amino acids 1-147 of the DNA binding domain of Saccharomyces cerevisiae GAL4 (GAL4 DBD) (SEQ ID NO: 16) and amino acid residues 729-984 of the ligand binding domain of human mineralocorticoid receptor (hMR LBD) (SEQ ID NO: 14). The expression vector contained an expression cassette of the nucleotide sequence for the GAL4 DBD (See SEQ ID NO: 19) ligated to nucleotides 2401 through 3168 of the human mineralocorticoid (hMR) receptor (SEQ ID NO: 13). Constitutive expression of this fusion protein is under control of a TATA box and the strong CMV promoter. The complete amino acid sequence for the expressed fusion protein (GAL4 DBD-hMR LBD) is set forth in FIG. 1 (SEQ ID NO: 19).

Reporter gene vector: The Galp3TKLuc reporter gene vector contained 3 tandem repeats of the 17 base pair 17M GAL4 DNA binding site (5′-CGG AGT ACT GTC CTC CG-3′; SEQ ID NO: 18) upstream of the basal human thymidine kinase promoter, and thus controlling the transcription of a luciferase reporter gene.

In vitro cell culture: CV-1 cells (African green monkey kidney cell line, ATCC accession number CCL-70) were maintained in culture in 90% DMEM medium supplemented by 9% FCS and 1% Penicillin-Streptomycin-L-Glutamine at 37° C. under 8.5% CO₂.

These cells were transiently co-transfected with the GAL4 DBD-hMR LBD fusion protein expression vector and the Galp3TKLuc reporter gene vector using the FuGENE 6 Transfection Reagent (Roche) according to the manufacturer's instructions.

Cell-Based Luciferase Assay:

Antagonist assay: The cells were incubated for 15 min at room temperature (RT) with test compound. Then 10 nM aldosterone (Acros Organics, catalogue # 215360050) was added. In some cases, 2 nM aldosterone was used. As a positive control, 90% DMEM medium supplemented by 9% charcoal-treated FCS and 1% Penicillin-Streptomycin-L-Glutamine containing 1% DMSO plus 0.001% ethanol was used in the absence of aldosterone. For negative controls, no further additions besides 20 nM aldosterone were used. The samples were incubated for 24 hours at 37° C. under 8.5% CO₂ before the Luciferase substrate (Promega, catalogue number #E2620) was added. After an incubation period of 5 min at RT, luminescence signals were read on a microplate reader (Molecular Devices/LJL Biosystems). For quantitation of an IC₅₀, the experiment was carried out using varying concentrations of test compounds. IC₅₀ values were determined as described above (see Example 67: In vitro competitive binding assays). For test compounds, the reported IC₅₀ values represent the mean value calculated from the results of 1-3 experiments carried out in duplicate. For eplerenone and spironolactone, the reported IC₅₀ values represent the mean value calculated from the results of more than 10 experiments each.

Agonist assay: As a positive control, aldosterone (Acros Organics, catalogue # 215360050) was used at a concentration of 100 nM. The negative control sample was 90% DMEM medium supplemented by 9% charcoal-treated FCS and 1% Penicillin-Streptomycin-L-Glutamine containing 0.2% DMSO plus 0.02% ethanol. Positive control, negative control, or 10 μM final concentration of test compound was added to the cells and incubated for 24 hours at 37° C. under 8.5% CO₂ before the Luciferase substrate (Promega, catalogue number #E2620) was added. After an incubation period of 5 min at RT, luminescence signals were read on a microplate reader (Molecular Devices/LJL Biosystems). Negative and positive controls were defined as 0% and 100% agonism, respectively. The agonistic potency of test compounds is given in % and was normalized to the positive and negative controls. For test compounds the reported % agonism values represent the mean value calculated from the results of 1-3 experiments. For eplerenone and spironolactone the reported % agonism values represent the mean value calculated from the results of more than 10 experiments each.

IC₅₀ ratio calculations: To provide a measure of the potency of each test compound as an antagonist of MR, the IC₅₀ of each test compound (IC₅₀^{Test}) for aldosterone induction of luciferase expression in the cell based assay, and the IC₅₀ for aldosterone induction of luciferase expression for eplerenone (IC₅₀^{Ep}) and spironolactone (IC₅₀^{Sp}), were used to calculate an IC₅₀ ratio (RIC₅₀) according to the following equations:
RIC₅₀^{Ep/Test} =IC₅₀^{Ep}/IC₅₀^{Test} and
RIC₅₀^{SP/Test} =IC₅₀^{Sp}/IC₅₀^{Test}.
B. Results and Discussion

The mineralocorticoid receptor (MR) antagonist compounds of the invention were further characterized in in vitro functional assays. These assays were used to quantitate the ability of the compounds to antagonize aldosterone-induced, GAL4 DBD-MR LBD fusion protein-dependent expression of a luciferase reporter gene. For these assays, a fusion protein of the DNA binding domain of the Saccharomyces cerevisiae transcription factor GAL4 and amino acid residues 729-984 of the human mineralocorticoid ligand binding domain (GAL4 DBD-hMR LBD fusion protein) was expressed in CV-1 cells. These cells also harboured a luciferase reporter gene whose expression is regulated by 3 tandem repeats of the GAL4 DBD target sequence (a.k.a. “17M”). Thus, upon agonist (e.g., aldosterone) binding to the hMR LBD of the GAL4 DBD-hMR LBD fusion protein, the fusion protein binds to the GAL4 DBD target sequence. As a consequence of DNA binding, expression of the luciferase reporter gene is activated by the transactivation activity of the hMR LBD.

To provide a measure of the potency of each test compound, IC₅₀ ratios (RIC₅₀) were calculated based upon the IC₅₀ of each test compound for inhibition of aldosterone induction of luciferase expression in the cell based assay, and the IC₅₀ of eplerenone or spironolactone for inhibition of aldosterone induction of luciferase expression in the same cell based assay.

The results of such experiments show that the compounds of formula I are MR antagonists. Some of the compounds of formula I, including Examples 16, 24, 27, 33, 36, 38, 44, 46, 53, 58, 59, 75, 76, 77, 82A, 82B and 84 are more potent MR antagonists than eplerenone (RIC₅₀^{Ep/Test}≧1.0), while some of the compounds of formula I, including Examples 11, 70, 71 and 83 are more than an order of magnitude more potent than eplerenone (RIC₅₀^{Ep/Test}≧10.0). Similarly, some of the compounds of formula I, including Example 23, 40, and 42, are comparable to spironolactone in potency (RIC₅₀^{Sp/Test}≧0.5), while some of the compounds of formula I, including Example 7, 8, 9, 10, 12, 13, 18, 26, 34, 41, 52, 70, 71 and 83 are more potent MR antagonists than spironolactone (RIC₅₀^{Sp/Test}≧1.0).

In addition, the novel compounds of the invention do not significantly activate the MR (i.e., are very weak MR agonists). The ability of 10 μM of test compound to activate luciferase expression in the cell based assay was determined and expressed as percent agonism relative to the amount of luciferase expression observed with 10 nM aldosterone (defined as 100% agonism). Note that the amount of test compound applied was three orders of magnitude greater than the amount of aldosterone applied. The observed % agonism for the novel compounds of the invention was generally 10% or less, indicating that the novel compounds of the invention are generally at least four orders of magnitude less potent than aldosterone for activation of MR. These results are consistent with the observed % agonism for 10 μM of eplerenone and spironolactone (also <10%).

Thus, preferred compounds of the invention are potent MR antagonists. Furthermore, the preferred compounds of the invention are not, in and of themselves, potent MR agonists.

Thus, preferred compounds of the invention, including Examples 7, 8, 9, 10, 12, 13, 18, 26, 34, 41, 52, 70, 71 and 83 are more potent (e.g., as shown by RIC₅₀^{Ep/Test}≧1.0 and RIC₅₀^{Sp/Test}≧1.0) than both eplerenone and spironolactone, MR antagonists previously described in the art. Furthermore, these preferred compounds are not, in and of themselves, potent MR agonists (e.g., at least three orders of magnitude less potent than aldosterone for activation of MR).

### Example 87

### In Vivo Functional Assays

Methods

To evaluate the in vivo activity of test compounds as aldosterone receptor antagonists, the Kagawa model was used, with some modifications. The original model first described by Kagawa (Endocrinology 1960; 67:125-131) has been widely utilised and reported in several published papers. Spironolactone and eplerenone have previously been shown to be aldosterone receptor antagonists using the Kagawa test (see, for example, de Gasparo, et al. J Pharmacol Exp Ther 1987; 240:650-656).

Bilateral adrenalectomised male Spague-Dawley rats aging approximately 5 weeks and weighing between 150 and 170 g at the time of surgery were obtained from Charles River (UK) Ltd., Margate, Kent. Following adrenalectomy, animals were maintained with standard diet and 0.9% (w/v) saline to drink. Saline replaces sodium ions lost due to the absence of the adrenal glands.

The animals were maintained for three days in standard condition and were trained in metabolic cages on one occasion prior to the test day for approximately 5 hours.

Food was withheld from animals overnight prior to dosing. Saline was available ad libitum.

Approximately 2 hours before dosing, saline was removed from the animals to ensure similar hydration levels.

Each animal received a single administration of vehicle, test compound or reference compound by oral gavage (treatment 1), using a constant dose volume of 10 mL/kg. Individual dose volumes were based on the individual body weights of animals obtained on their respective day of dosing.

Ten minutes later, each animal received a 0.5 mL subcutaneous injection of aldosterone or saline (treatment 2), immediately followed by a loading dose of 0.9% (w/v) saline by oral gavage using a constant dose volume of 25 mL/kg. This dose of saline served to increase the urine output.

The vehicle control and vehicle for the test compound and reference compound (spironolactone or eplerenone) was identified as 0.5% (w/v) carboxymethylcellulose (CMC). The vehicle for aldosterone was 0.9% (w/v) saline.

Immediately after dosing the animals were placed in individual metabolic cages.

The cumulative urine volume was measured and collected for analysis at 0-3 and 3-6 hours post-aldosterone. Urine was analysed for urinary sodium and potassium concentration.

During the urine collection period, the animals were deprived of food and saline.

Treatment groups employed for the study contained at least 8 animals each. Compounds were tested at two dosage levels each.

### Statistical Analysis

Mean Na⁺:K⁺ ratios from each group were calculated. The vehicle control group (Group 1) was taken as the baseline group with which each of the treated groups was compared.

The data were analysed using one-way Analysis of Variance (ANOVA). Levene's test was used to test for equality of variances between the groups. Where Levene's was non-significant (P≧0.01), pairwise comparisons with vehicle control was made using DUNNETT'S test. A log transformation may be applied to achieve variance stability.

Tests were performed using a two-sided risk. Significant results were reported as P<0.05, P<0.01 or P<0.001.

Where the data proved unsuitable for this procedure, non-parametric methods were used. The non-parametric methods employed were the Kruskal-Wallis non-parametric ANOVA, protected Wilcoxon Rank Sum test and Terpstra-Jonckheere test for a dose-response.

Results

In adrenalectomized rats, the subcutaneous administration of aldosterone at 3 μg/animal induced a significant reduction of Na⁺/K⁺ ratio, due to reduction in Na⁺ and increase in K⁺ excretion in the urine collected after administration. The compounds of Example 7 and Example 13 were tested for their activity to antagonize the effect of aldosterone on urinary Na⁺/K⁺ ratio in this model. Both compounds provided statistically significant increase in urinary Na⁺/K⁺ relative to animals only receiving aldosterone (i.e., no test compound). Thus, the results of this assay show that the compounds of the invention effectively antagonize the effects of aldosterone in vivo.

In describing particular embodiments of the present invention, specific terminology is employed for the sake of clarity. However, the invention is not intended to be limited to the specific terminology so selected, and it is to be understood that each specific element includes all technical equivalents which operate in a similar manner to accomplish a similar purpose.

The embodiments illustrated and discussed in this specification are intended only to teach those skilled in the art the best way known to the inventors to make and use the invention. Nothing in this specification should be considered as limiting the scope of the present invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

The present invention is not limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values given in the foregoing examples are approximate, and are provided for purposes of illustration.

Patents, patent applications, publications, product descriptions, and protocols which are cited throughout this application are incorporated herein by reference in their entireties for all purposes.

What is claimed is:

1. A compound of formula I: 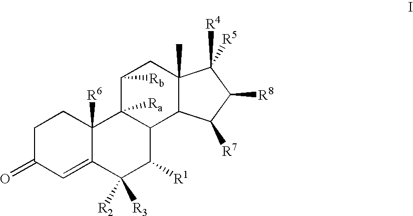
wherein
R¹ is selected from hydrogen, alkyl, alkenyl, alkynyl, cycloalkyl, carboxyl, cyano, tetrazolyl, isoxadiazolyl, alkylisoxadiazolyl, cycloalkylthio, —C(═O)O-alkyl, —C(═O)-alkyl, —C(═O)NHR^{x}, —C(═O)N(alkyl)(R^{x}), —C═N—OR^{x}, —C═N—O(aralkyl), —C(═O)N(R^{x})(O-alkyl), —CH₂OR^{x}, —CH₂NHR^{x}, —CH₂N(alkyl)(R^{x}), OR^{x}, OC(═O)NH-alkyl, —OC(═O)N(alkyl)₂, OC(═O)alkyl, —SR^{x}, —SC(═O)-alkyl, —S(═O)-alkyl, —SO₂-alkyl, —S-alkyl-C(═O)O-alkyl, —S-alkyl-N(R^{y})C(═O)-alkyl, —S-alkyl-CF₃, —S-alkyl-N(R^{y})₂, —NHR^{x}, —N(alkyl)(R^{x}), —N(R^{y})C(═O)-alkyl, —N(R^{y})C(═O)O-alkyl, —N(R^{y})—C(═O)NHR^{x}, —N(R^{y})—SO₂-alkyl, and —N—(R^{y})C(═O)—CF₃;
R² is selected from hydrogen and halogen;
R³ is hydrogen or hydroxymethyl (CH₂OH), or
R² and R³ when taken together with the carbon to which they are attached form a cyclopropyl ring system
R⁴ and R⁵ when taken together form a ring system selected from 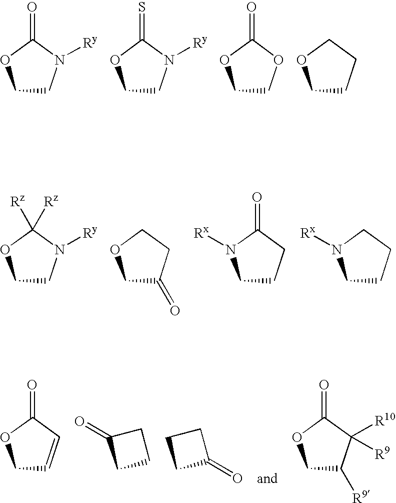
Ra and Rb are not present and there is a C═C between carbons 9 and 11 or;
Ra and Rb together form a bridge A between carbons 9 and 11, wherein
A is selected from —O—, —S—, —CH₂—, and —CF₂—;
R⁶ is selected from hydrogen, alkyl, CH₂OR^{x}, —CH₂SR^{x}, —CH₂SO-alkyl, —CH₂SO₂-alkyl, —CH₂NHR^{x}, —CH₂N(alkyl)(R^{x}), —C(═O)O-alkyl, —C(═O)-alkyl, —C(═O)NHR^{x}, and C(═O)N(alkyl)(R^{x});
R⁷ and R⁸ are hydrogen or when taken together with the carbon to which they are attached form a cyclopropyl ring system
R⁹ and R^{9′} are independently selected from hydrogen, halo, alkyl, and —C(═O)O-alkyl;
or R⁹ and R^{9′} are not present and there is a C═C between carbons 22 and 23
R¹⁰ is selected from hydrogen and C(═O)OR^{x};
R^{x} is selected from hydrogen, alkyl, and acyl;
R^{y} is selected from hydrogen and alkyl;
R^{z} is selected from hydrogen and alkyl;
wherein
when R¹ contains an alkyl or alkenyl substituent, said alkyl or alkenyl substituent in R¹ can optionally and independently be substituted by one or more of —OR^{x}, NHR^{x}, —N(alkyl)(R^{x}), halogen, —C(═O)N(R^{y})₂, —N(R^{y})₂, aryl, —C(═O)—Oalkyl, OC(═O)NHR^{x}, —OC(═O)alkyl, —OC(═O)Oalkyl, OC(═O)alkyl-O-alkyl, and OC(═O)alkyl-O—Ac; and
pharmaceutically acceptable salts, solvates, prodrugs and metabolites thereof;
with the following provisos: (i) R¹ is not —C(═O)O-alkyl, —C(═O)OH or hydrogen when R² is hydrogen, Ra and Rb form an oxo bridge or are not present and there is a C═C between carbons 9 and 11 and R⁴ and R⁵ together form 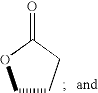
(ii) R¹ is not —S—C(═O)—CH₃ when R² is hydrogen, Ra and Rb form an oxo bridge and R⁴ and R⁵ together form 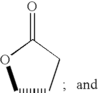
(iii) R¹ is not cyano when R² is hydrogen, Ra and Rb are not present and there is a C═C between carbons 9 and 11, and R⁴ and R⁵ together form 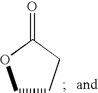
(iv) R¹ is not hydrogen when R² is hydrogen, Ra and Rb form an oxo bridge or are not present and there is a C═C between carbons 9 and 11, and R4 and R5 together form 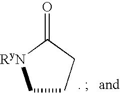
(v) Ra and Rb do not together form an oxo bridge or there is not a C═C between carbons 9 and 11 when R¹ is hydrogen or alkanoylthio, R² is hydrogen, and R⁴ and R⁵ together form 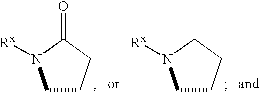
(vi) R¹ is not 2-propenyl or 3-butenyl when R² is hydrogen, Ra and Rb are not present and there is a C═C between carbons 9 and 11, and R4 and R5 together form

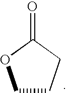

2. The compound of claim 1, where the aldosterone receptor antagonist is a selective aldosterone receptor antagonist.

3. The compound of claim 1, wherein
R¹ is selected from hydrogen, alkyl, alkenyl, carboxyl, cyano, alkylisoxadiazolyl, cycloalkylthio, —C(═O)O-alkyl, —C(═O)-alkyl, —C(═O)NHR^{x}, —C(═O)N(alkyl)(R^{x}), —C═N—OR^{x}, —C(═O)N(R^{x})(O-alkyl), —CH₂OR^{x}, —CH₂NHR^{x}, —CH₂N(alkyl)(R^{x}), —SR^{x}, —SC(═O)-alkyl, —S(═O)-alkyl, —SO₂-alkyl, —S-alkyl-C(═O)O-alkyl, —S-alkyl-N(R^{y})C(═O)-alkyl, —S-alkyl-CF₃, —S-alkyl-N(R^{y})₂, —NHR^{x}, —N(alkyl)(R^{x}), —N(R^{y})C(═O)-alkyl, —N(R^{y})C(═O)O-alkyl, —N(R^{y})—C(═O)NHR^{x}, —N(R^{y})—SO₂-alkyl, and —N—(R^{y})C(═O)—CF₃;
R² is selected from hydrogen and halogen;
R³ is hydrogen or hydroxymethyl (CH₂OH); or
R2 and R3 taken together with the carbon to which they are attached form a cycloproyl bridge,
R⁴ and R⁵ when taken together form a ring system selected from 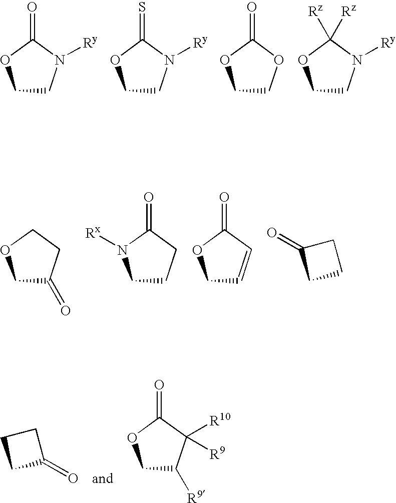
Ra and Rb are not present and there is a C═C between carbons 9 and 11 or;
Ra and Rb together form a bridge A between carbons 9 and 11, wherein
A is —O—;
R⁶ is alkyl
R⁷ and R⁸ are hydrogen or when taken together with the carbon to which they are attached form a cyclopropyl ring system,
R⁹ and R^{9′} are independently selected from hydrogen, halo, alkyl, and —C(═O)O-alkyl;
or R⁹ and R^{9′} are not present and there is a C═C between carbons 22 and 23
R¹⁰ is selected from hydrogen and —C(═O)OR^{x};
R^{x} is selected from hydrogen, alkyl, and acyl;
R^{y} is selected from hydrogen and alkyl;
R^{z} is alkyl;
wherein
when R¹ contains an alkyl or alkenyl substituent, said alkyl or alkenyl substituent in R¹ can optionally and independently be substituted by one or more of —OR^{x}, —NHR^{x}, —N(alkyl)(R^{x}), halogen, —C(═O)N(R^{y})₂, —N(R^{y})₂, phenyl, OC(═O)NHR^{x}, —OC(═O)alkyl, OC(═O)alkyl-O-alkyl, and OC(═O)alkyl-O—Ac.

4. A method of inhibiting aldosterone receptor activity comprising contacting an aldosterone receptor with an effective amount of a compound of any one of claims 1 to 3.

5. The method of claim 4, wherein the aldosterone receptor is a mammalian aldosterone receptor.

6. The method of claim 5, wherein the aldosterone receptor is a human aldosterone receptor.

7. A method of treating aldosteronism comprising administering to a patient in need of such treatment an effective amount of a compound of any one of claims 1 to 3.

8. The method of claim 7, wherein the aldosteronism is selected from the group consisting of primary hyperaldosteronism and secondary hyperaldosteronism.

9. The method of claim 7, wherein the aldosteronism is selected from the group consisting of hypertension, cardiovascular disease, renal dysfunction, edema, cerebrovascular disease, and insulinopathies.

10. The method of claim 9, wherein the aldosteronism is selected from the group consisting of hypertension, cardiovascular disease, stroke, and Type II diabetes mellitus.

11. The method of claim 10, wherein the cardiovascular disease is selected from the group consisting of heart failure and left ventricular hypertrophy.

12. The method of claim 7, wherein the patient is a mammal.

13. The method of claim 12, wherein the patient is a human.

14. A pharmaceutical composition comprising a compound of any one of claims 1 to 3 and, optionally, a pharmaceutically acceptable carrier.

15. The pharmaceutical composition of claim 14 further comprising a second active agent.

16. The pharmaceutical composition of claim 15, wherein the second active agent is selected from the group consisting of renin inhibitors, angiotensin II antagonists, ACE inhibitors, diuretics, and retinoic acid.


SEQUENCE LISTING (18 sequences)

SEQ ID NO: 1 - DNA, 5749 bp, Homo sapiens
cgcgggagccaacttcaggctgctcagaggaagcccgtgcagtcagtcacctgggtgcaagagcgttgctgcctcgggctctcccgctgcagggagagcggcactcgctggcctggatgtggttggatttaggggggctccgcagcaggggtttcgtggcggtggcaagcgctgcaacaggtagacggcgagagacggaccccggccgaggcagggatggagaccaaaggctaccacagtctccctgaaggtctagatatggaaagacggtggggtcaagtttctcaggctgtggagcgttcttccctgggacctacagagaggaccgatgagaataactacatggagattgtcaacgtaagctgtgtttccggtgctattccaaacaacagtactcaaggaagcagcaaagaaaaacaagaactactcccttgccttcagcaagacaataatcggcctgggattttaacatctgatattaaaactgagctggaatctaaggaactttcagcaactgtagctgagtccatgggtttatatatggattctgtaagagatgctgactattcctatgagcagcagaaccaacaaggaagcatgagtccagctaagatttatcagaatgttgaacagctggtgaaattttacaaaggaaatggccatcgtccttccactctaagttgtgtgaacacgcccttgagatcatttatgtctgactctgggagctccgtgaatggtggcgtcatgcgcgccattgttaaaagccctatcatgtgtcatgagaaaagcccgtctgtttgcagccctctgaacatgacatcttcggtttgcagccctgctggaatcaactctgtgtcctccaccacagccagctttggcagttttccagtgcacagcccaatcacccagggaactcctctgacatgctcccctaatgctgaaaatcgaggctccaggtcgcacagccctgcacatgctagcaatgtgggctctcctctctcaagtccgttaagtagcatgaaatcctcaatttccagccctccaagtcactgcagtgtaaaatctccagtctccagtcccaataatgtcactctgagatcctctgtgtctagccctgcaaatattaacaactcaaggtgctctgtttccagcccttcgaacactaataacagatccacgctttccagtccggcagccagtactgtgggatctatctgtagccctgtaaacaatgccttcagctacactgcttctggcacctctgctggatccagtacattgcgggatgtggttcccagtccagacacgcaggagaaaggtgctcaagaggtcccttttcctaagactgaggaagtagagagtgccatctcaaatggtgtgactggccagcttaatattgtccagtacataaaaccagaaccagatggagcttttagcagctcatgtctaggaggaaatagcaaaataaattcggattcttcattctcagtaccaataaagcaagaatcaaccaagcattcatgttcaggcacctcttttaaagggaatccaacagtaaacccgtttccatttatggatggctcgtatttttcctttatggatgataaagactattattccctatcaggaattttaggaccacctgtgcccggctttgatggtaactgtgaaggcagcggattcccagtgggtattaaacaagaaccagatgacgggagctattacccagaggccagcatcccttcctctgctattgttggggtgaattcaggtggacagtccttccactacaggattggtgctcaaggtacaatatctttatcacgatcggctagagaccaatctttccaacacctgagttcctttcctcctgtcaatactttagtggagtcatggaaatcacacggcgacctgtcgtctagaagaagtgatgggtatccggtcttagaatacattccagaaaatgtatcaagctctactttacgaagtgtttctactggatcttcaagaccttcaaaaatatgtttggtgtgtggggatgaggcttcaggatgccattatggggtagtcacctgtggcagctgcaaagttttcttcaaaagagcagtggaagggcaacacaactatttatgtgctggaagaaatgattgcatcattgataagattcgacgaaagaattgtcctgcttgcagacttcagaaatgtcttcaagctggaatgaatttaggagcacgaaagtcaaagaagttgggaaagttaaaagggattcacgaggagcagccacagcagcagcagcccccacccccacccccacccccgcaaagcccagaggaagggacaacgtacatcgctcctgcaaaagaaccctcggtcaacacagcactggttcctcagctctccacaatctcacgagcgctcacaccttcccccgttatggtccttgaaaacattgaacctgaaattgtatatgcaggctatgacagctcaaaaccagatacagccgaaaatctgctctccacgctcaaccgcttagcaggcaaacagatgatccaagtcgtgaagtgggcaaaggtacttccaggatttaaaaacttgcctcttgaggaccaaattaccctaatccagtattcttggatgtgtctatcatcatttgccttgagctggagatcgtacaaacatacgaacagccaatttctctattttgcaccagacctagtctttaatgaagagaagatgcatcagtctgccatgtatgaactatgccaggggatgcaccaaatcagccttcagttcgttcgactgcagctcacctttgaagaatacaccatcatgaaagttttgctgctactaagcacaattccaaaggatggcctcaaaagccaggctgcatttgaagaaatgaggacaaattacatcaaagaactgaggaagatggtaactaagtgtcccaacaattctgggcagagctggcagaggttctaccaactgaccaagctgctggactccatgcatgacctggtgagcgacctgctggaattctgcttctacaccttccgagagtcccatgcgctgaaggtagagttccccgcaatgctggtggagatcatcagcgaccagctgcccaaggtggagtcggggaacgccaagccgctctacttccaccggaagtgactgcccgctgcccagaagaactttgccttaagtttccctgtgttgttccacacccagaaggacccaagaaaacctgtttttaacatgtgatggttgattcacacttgttcaacagtttctcaagtttaaagtcatgtcagaggtttggagccgggaaagctgtttttccgtggatttggcgagaccagagcagtctgaaggattccccacctccaatcccccagcgcttagaaacatgttcctgttcctcgggatgaaaagccatatctagtcaataactctgattttgatattttcacagatggaagaagttttaactatgccgtgtagtttctggtatcgttcgcttgttttaaaagggttcaaggactaacgaacgttttaaagcttacccttggtttgcacataaaacgtatagtcaatatggggcattaatattcttttgttattaaaaaaacacaaaaaaataataaaaaaatatatacagattcctgttgtgtaataacagaactcgtggcgtggggcagcagctgcctctgagccctcgctcgtccacggtcttctgcatcactggtatacacactcgttagcgtccatttcttatttaattagaatggataagatgatgttaaatgccttggtttgatttctagtatctattgtgttggctttacaaataattttttgcagtcttttgctgtgctgtacattactgtatgtataaattatgaaggacctgaaataaggtataaggatcttttgtaaatgagacacatacaaaaaaaatctttaatggttaataggatgaatgggaaagtatttttgaaagaattctattttgctggagactatttaagtactatctttgtctaaacaaggtaatttttttttgtaaagtgcaatgtcctgcatgcataatgaaccgtttacagtgtatttaagaaagggaaagctgtgccttttttagcttcatatctaatttaccattattttacagtctctgttgtaaataaccacactgaaacctcttcggttgtcttgaaacctttctactttttctgtactttttgttttgttcttggtctcccgcttggggcatttgtgggactccagcacgttttctggcttctgcttcatcctgctccatcggggaatgacacactgcggtgtctgcagctcctggaaggtgtcatttgacaacacatgtgggagaggaggtccttggagtgctgcagctttgggaaagcctgcctcgtttcccttttcctctagaagcagaaccagctctacgagagtgagactgggaacttgatggctcagagagcatcttttcctcccattttagaaaatcagattttctcctgtgggaaaaaaaaattccatgcactctctctctgttaaagatcagctattcccttctgatcttggaaagaggttctgcactcctggaaccggtcacaggaacgcacagatcatggcaggatgcgctgggacggcccatcttggcaaggttcagtctgaatggcatggagaccgggagatagaggggttttagatttttaaaaggtaggttttaaaaataagttttatacataaacagttttggagaaaaattacagatcatataagcaagacagtggcactaaaatgtttaattcattaatctgtttgtttggcactgatgcaatgtatggcttttctcttgccccaaatcacaaacatatgtatctttggggaaactaacaatatgattgcactaaataaactactttgaatagaggccaaattaatcttttaaaaatgatgataatcatcaggtttactcagtgaaatcatattaattattttccaaaatctaaaagctgtagctggagaagcccatggccacgaggaagcagcaattaattagatcaacacttttctccagggttcaccatgcaggcaacattaccttgtctttcaaaagacacctgccttagtgcaaggggaaacctgtgaaagctgcactcagagggaggagtctttcttacataatttgcaatttcaggaatttaatttataggcagatctttaaatacagtcaacttacggtgcacagtaatatgaaagccacactttgaaggtaataaatacacagcatgcagactgggagttgctagcaaacaaatggcttacttacaaaagcagcttttagttcagacttagtttttataaaatgagaattctgacttacttaaccaggtttgggatggagatggtctgcatcagctttttgtattaacaaagttactggctctttgtgtgtctccaggtaactttgcttgattaaacagcaaagccatattctaaattcactgttgaatgcctgtcccagtccaaattgtctgtctgctcttatttttgtaccatattgctcttaaaaatcttggtttggtacagttcataattcaccaaaaagttcatataatttaaagaaacactaaattagtttaaaatgaagcaatttatatctttatgcaaaaacatatgtctgtctttgcaaaggactgtaagcagattacaataaatcctttacttt

SEQ ID NO: 2 - protein, 984 aa, Homo sapiens
METKGYHSLPEGLDMERRWGQVSQAVERSSLGPTERTDENNYMEIVNVSCVSGAIPNNSTQGSSKEKQELLPCLQQDNNRPGILTSDIKTELESKELSATVAESMGLYMDSVRDADYSYEQQNQQGSMSPAKIYQNVEQLVKFYKGNGHRPSTLSCVNTPLRSFMSDSGSSVNGGVMRAIVKSPIMCHEKSPSVCSPLNMTSSVCSPAGINSVSSTTASFGSFPVHSPITQGTPLTCSPNAENRGSRSHSPAHASNVGSPLSSPLSSMKSSISSPPSHCSVKSPVSSPNNVTLRSSVSSPANINNSRCSVSSPSNTNNRSTLSSPAASTVGSICSPVNNAFSYTASGTSAGSSTLRDVVPSPDTQEKGAQEVPFPKTEEVESAISNGVTGQLNIVQYIKPEPDGAFSSSCLGGNSKINSDSSFSVPIKQESTKHSCSGTSFKGNPTVNPFPFMDGSYFSFMDDKDYYSLSGILGPPVPGFDGNCEGSGFPVGIKQEPDDGSYYPEASIPSSAIVGVNSGGQSFHYRIGAQGTISLSRSARDQSFQHLSSFPPVNTLVESWKSHGDLSSRRSDGYPVLEYIPENVSSSTLRSVSTGSSRPSKICLVCGDEASGCHYGVVTCGSCKVFFKRAVEGQHNYLCAGRNDCIIDKIRRKNCPACRLQKCLQAGMNLGARKSKKLGKLKGIHEEQPQQQQPPPPPPPPQSPEEGTTYIAPAKEPSVNTALVPQLSTISRALTPSPVMVLENIEPEIVYAGYDSSKPDTAENLLSTLNRLAGKQMIQVVKWAKVLPGFKNLPLEDQITLIQYSWMCLSSFALSWRSYKHTNSQFLYFAPDLVFNEEKMHQSAMYELCQGMHQISLQFVRLQLTFEEYTIMKVLLLLSTIPKDGLKSQAAFEEMRTNYIKELRKMVTKCPNNSGQSWQRFYQLTKLLDSMHDLVSDLLEFCFYTFRESHALKVEFPAMLVEIISDQLPKVESGNAKPLYFHRK

SEQ ID NO: 3 - DNA, 5003 bp, Homo sapiens
ggatccattttataagctcaaagataattacttttcagactaagaatatttagggtaaaaagtactgttcaacatctctactgaggatgttatgatgtagcacactctataagctggagctaaaggaaactttccttaaagtgctatttactaaaaattggaacacattccttaagacaaatcgaagtgtggcacacaacatccaaacttccatcatagatacagaggtgttaccatctcccactcccaaatttctttgtcacgctgaggatactcaagaggagcaggacatgttggtcgcagcaggagaaacttgaaagcattcacttttatggaactcataagggagagaatctcttatttagtatcgtccttgatacatttattattttaaaagataatgtagccaaatgtcttcctctgtgttaaatctttacaaaactgaaatcttaaaatggtgacaaaaattctacttctgatagaatctattcatttttccaattagatagggcataattcttaatttgcaaaacaaaacgtaatatgcttatgaggttccatcccaaagaacctgctattgagagtagcattcagaataacgggtggaaatgccaactccagagtttcagatcctaccggtaattggggtagggaggggctttgggcggggcctccctagaggaggaggcgttgttagaaagctgtctggccagtccacagctgtcactaatcggggtaagccttgttgtatttgcgcgtgtgggtggcattctcaatgagaactagcttcacttgtcatttgagtgaaatctacaacccgaggcggctagtgctcccgcactactgggatctgagatcttcggagatgactgtcgcccgcagtacggagccagcagaagtccgacccttcctgggaatgggctgtaccgagaggtccgactagccccagggttttagtgagggggcagtggaactcagcgagggactgagagcttcacagcatgcacgagtttgatgccagagaaaaagtcgggagataaaggagccgcgtgtcactaaattgccgtcgcagccgcagccactcaagtgccggacttgtgagtactctgcgtctccagtcctcggacagaagttggagaactctcttggagaactccccgagttaggagacgagatctcctaacaattactactttttcttgcgctccccacttgccgctcgctgggacaaacgacagccacagttcccctgacgacaggatggaggccaagggcaggagctgaccagcgccgccctcccccgcccccgacccaggaggtggagatcctccggtccagccacattcaacacccactttctcctccctctgcccctatattcccgaaaccccctcctccttcccttttccctcctccctggagacgggggaggagaaaaggggagtccagtcgtcatgactgagctgaaggcaaagggtccccgggctccccacgtggcgggcggcccgccctcccccgaggtcggatccccactgctgtgtcgcccagccgcaggtccgttcccggggagccagacctcggacaccttgcctgaagtttcggccatacctatctccctggacgggctactcttccctcggccctgccagggacaggacccctccgacgaaaagacgcaggaccagcagtcgctgtcggacgtggagggcgcatattccagagctgaagctacaaggggtgctggaggcagcagttctagtcccccagaaaaggacagcggactgctggacagtgtcttggacactctgttggcgccctcaggtcccgggcagagccaacccagccctcccgcctgcgaggtcaccagctcttggtgcctgtttggccccgaacttcccgaagatccaccggctgcccccgccacccagcgggtgttgtccccgctcatgagccggtccgggtgcaaggttggagacagctccgggacggcagctgcccataaagtgctgccccggggcctgtcaccagcccggcagctgctgctcccggcctctgagagccctcactggtccggggccccagtgaagccgtctccgcaggccgctgcggtggaggttgaggaggaggatagctctgagtccgaggagtctgcgggtccgcttctgaagggcaaacctcgggctctgggtggcgcggcggctggaggaggagccgcggcttgtccgccgggggcggcagcaggaggcgtcgccctggtccccaaggaagattcccgcttctcagcgcccagggtcgccctggtggagcaggacgcgccgatggcgcccgggcgctccccgctggccaccacggtgatggatttcatccacgtgcctatcctgcctctcaatcacgccttattggcagcccgcactcggcagctgctggaagacgaaagttacgacggcggggccggggctgccagcgcctttgccccgccgcggacttcaccctgtgcctcgtccaccccggtcgctgtaggcgacttccccgactgcgcgtacccgcccgacgccgagcccaaggacgacgcgtaccctctctatagcgacttccagccgcccgctctaaagataaaggaggaggaggaaggcgcggaggcctccgcgcgctccccgcgttcctaccttgtggccggtgccaaccccgcagccttcccggatttcccgttggggccaccgcccccgctgccgccgcgagcgaccccatccagacccggggaagcggcggtgacggccgcacccgccagtgcctcagtctcgtctgcgtcctcctcggggtcgaccctggagtgcatcctgtacaaagcggagggcgcgccgccccagcagggcccgttcgcgccgccgccctgcaaggcgccgggcgcgagcggctgcctgctcccgcgggacggcctgccctccacctccgcctctgccgccgccgccggggcggcccccgcgctctaccctgcactcggcctcaacgggctcccgcagctcggctaccaggccgccgtgctcaaggagggcctgccgcaggtctacccgccctatctcaactacctgaggccggattcagaagccagccagagcccacaatacagcttcgagtcattacctcagaagatttgtttaatctgtggggatgaagcatcaggctgtcattatggtgtccttacctgtgggagctgtaaggtcttctttaagagggcaatggaagggcagcacaactacttatgtgctggaagaaatgactgcatcgttgataaaatccgcagaaaaaactgcccagcatgtcgccttagaaagtgctgtcaggctggcatggtccttggaggtcgaaaatttaaaaagttcaataaagtcagagttgtgagagcactggatgctgttgctctcccacagccattgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacatgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacagcttcatctgtactgcttgaatacatttatccagtcccgggcactgagtgttgaatttccagaaatgatgtctgaagttattgctgcacaattacccaagatattggcagggatggtgaaaccccttctctttcataaaaagtgaatgtcatctttttcttttaaagaattaaattttgtggcatgtctttttgttttggtcaggattatgaggtcttgagtttttataatgttcttctgaaagccttacatttataacatcatagtgtgtaaatttaaaagaaaaattgtgaggttctaattattttcttttataaagtataattagaatgtttaactgttttgtttacccatattttcttgaagaatttacaagattgaaaaagtactaaaattgttaaagtaaactatatcttatccatattatttcataccatgtaggtgaggatttttaacttttgcatctaacaaatcatcgacttaagagaaaaaatcttacatgtaataacacaaagctattatatgttatttctaggtaactccctttgtgtcaattatatttccaaaaatgaacctttaaaatggtatgcaaaattttgtctatatatatttgtgtgaggaggaaattcataactttcctcagattttcaaaagtatttttaatgcaaaaaatgtagaaagagtttaaaaccactaaaatagattgatgttcttcaaactaggcaaaacaactcatatgttaagaccattttccagattggaaacacaaatctcttaggaagttaataagtagattcatatcattatacaaatagtattgtgggttttgtaggtttttaaaataaccttttttggggagagaattgtcctctaatgaggtattgcgagtggc

SEQ ID NO: 4 - protein, 933 aa, Homo sapiens [sequence omitted]

SEQ ID NO: 5 - DNA, 4314 bp, Homo sapiens
cgagatcccggggagccagcttgctgggagagcgggacggtccggagcaagcccagaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttaggctgcacgcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaacaagcaaac

SEQ ID NO: 6 - protein, 920 aa, Homo sapiens
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 7 - DNA, 6456 bp, Homo sapiens
gagttgtgcctggagtgatgtttaagccaatgtcagggcaaggcaacagtccctggccgtcctccagcacctttgtaatgcatatgagctcgggagaccagtacttaaagttggaggcccgggagcccaggagctggcggagggcgttcgtcctgggactgcacttgctcccgtcgggtcgcccggcttcaccggacccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatccgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcattccccctataggaatacaaggggcacacagggaaggcagatcccctagttggcaagactattttaacttgatacactgcagattcagatgtgctgaaagctctgcctctggctttccggtcatgggttccagttaattcatgcctcccatggacctatggagagcagcaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgaggcacagccagacttgctcagggtggccctgccacaggctgcagctacctaggaacattccttgcagaccccgcattgccctttgggggtgccctgggatccctggggtagtccagctcttcttcatttcccagcgtggccctggttggaagaagcagctgtcacagctgctgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgaacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtgagctgctcgggcttctcttggtatgtcttgtttggaaaagtggatttcattcatttctgattgtccagttaagtgatcaccaaaggactgagaatctgggagggcaaaaaaaaaaaaaaagtttttatgtgcacttaaatttggggacaattttatgtatctgtgttaaggatatgtttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatattgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccagggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaatagagtcatacagtagctcaaaaggcaaccataattctctttggtgcaggtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcttttttttgcatccaattgtgcctgaacttttaaaatatgtaaatgctgccatgttccaaacccatcgtcagtgtgtgtgtttagagctgtgcaccctagaaacaacatattgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaact

SEQ ID NO: 8 - protein, 595 aa, Homo sapiens [sequence omitted]

SEQ ID NO: 9 - DNA, 2011 bp, Homo sapiens
tttcagtttctccagctgctggctttttggacacccactcccccgccaggaggcagttgcaagcgcggaggctgcgagaaataactgcctcttgaaacttgcagggcgaagagcaggcggcgagcgctgggccggggagggaccacccgagctgcgacgggctctggggctgcggggcagggctggcgcccggagcctgagctgcaggaggtgcgctcgctttcctcaacaggtggcggcggggcgcgcgccgggagaccccccctaatgcgggaaaagcacgtgtccgcattttagagaaggcaaggccggtgtgtttatctgcaagccattatacttgcccacgaatctttgagaacattataatgacctttgtgcctcttcttgcaaggtgttttctcagctgttatctcaagacatggatataaaaaactcaccatctagccttaattctccttcctcctacaactgcagtcaatccatcttacccctggagcacggctccatatacataccttcctcctatgtagacagccaccatgaatatccagccatgacattctatagccctgctgtgatgaattacagcattcccagcaatgtcactaacttggaaggtgggcctggtcggcagaccacaagcccaaatgtgttgtggccaacacctgggcacctttctcctttagtggtccatcgccagttatcacatctgtatgcggaacctcaaaagagtccctggtgtgaagcaagatcgctagaacacaccttacctgtaaacagagagacactgaaaaggaaggttagtgggaaccgttgcgccagccctgttactggtccaggttcaaagagggatgctcacttctgcgctgtctgcagcgattacgcatcgggatatcactatggagtctggtcgtgtgaaggatgtaaggccttttttaaaagaagcattcaaggacataatgattatatttgtccagctacaaatcagtgtacaatcgataaaaaccggcgcaagagctgccaggcctgccgacttcggaagtgttacgaagtgggaatggtgaagtgtggctcccggagagagagatgtgggtaccgccttgtgcggagacagagaagtgccgacgagcagctgcactgtgccggcaaggccaagagaagtggcggccacgcgccccgagtgcgggagctgctgctggacgccctgagccccgagcagctagtgctcaccctcctggaggctgagccgccccatgtgctgatcagccgccccagtgcgcccttcaccgaggcctccatgatgatgtccctgaccaagttggccgacaaggagttggtacacatgatcagctgggccaagaagattcccggctttgtggagctcagcctgttcgaccaagtgcggctcttggagagctgttggatggaggtgttaatgatggggctgatgtggcgctcaattgaccaccccggcaagctcatctttgctccagatcttgttctggacagggatgaggggaaatgcgtagaaggaattctggaaatctttgacatgctcctggcaactacttcaaggtttcgagagttaaaactccaacacaaagaatatctctgtgtcaaggccatgatcctgctcaattccagtatgtaccctctggtcacagcgacccaggatgctgacagcagccggaagctggctcacttgctgaacgccgtgaccgatgctttggtttgggtgattgccaagagcggcatctcctcccagcagcaatccatgcgcctggctaacctcctgatgctcctgtcccacgtcaggcatgcgagtaacaagggcatggaacatctgctcaacatgaagtgcaaaaatgtggtcccagtgtatgacctgctgctggagatgctgaatgcccacgtgcttcgcgggtgcaagtcctccatcacggggtccgagtgcagcccggcagaggacagtaaaagcaaagagggctcccagaacccacagtctcagtga

SEQ ID NO: 10 - protein, 530 aa, Homo sapiens
MDIKNSPSSLNSPSSYNCSQSILPLEHGSIYIPSSYVDSHHEYPAMTFYSPAVMNYSIPSNVTNLEGGPGRQTTSPNVLWPTPGHLSPLVVHRQLSHLYAEPQKSPWCEARSLEHTLPVNRETLKRKVSGNRCASPVTGPGSKRDAHFCAVCSDYASGYHYGVWSCEGCKAFFKRSIQGHNDYICPATNQCTIDKNRRKSCQACRLRKCYEVGMVKCGSRRERCGYRLVRRQRSADEQLHCAGKAKRSGGHAPRVRELLLDALSPEQLVLTLLEAEPPHVLISRPSAPFTEASMMMSLTKLADKELVHMISWAKKIPGFVELSLFDQVRLLESCWMEVLMMGLMWRSIDHPGKLIFAPDLVLDRDEGKCVEGILEIFDMLLATTSRFRELKLQHKEYLCVKAMILLNSSMYPLVTATQDADSSRKLAHLLNAVTDALVWVIAKSGISSQQQSMRLANLLMLLSHVRHASNKGMEHLLNMKCKNVVPVYDLLLEMLNAHVLRGCKSSITGSECSPAEDSKSKEGSQNPQSQ

SEQ ID NO: 11 - DNA, 4788 bp, Homo sapiens
tttttagaaaaaaaaaatatatttccctcctgctccttctgcgttcacaagctaagttgtttatctcggctgcggcgggaactgcggacggtggcgggcgagcggctcctctgccagagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttgtcaaaaaaaaaaaaaaaaaagctcatattttgtatatatctgcttcagtggagaattatataggttgtgcaaattaacagtcctaactggtatagagcacctagtccagtgacctgctgggtaaactgtggatgatggttgcaaaagactaatttaaaaaataactaccaagaggccctgtctgtacctaacgccctatttttgcaatggctatatggcaagaaagctggtaaactatttgtctttcaggaccttttgaagtagtttgtataacttcttaaaagttgtgattccagataaccagctgtaacacagctgagagacttttaatcagacaaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgatggtctcagaaaccaaacagtttgctctaggggaagagggagatggagactggtcctgtgtgcagtgaaggttgctgaggctctgacccagtgagattacagaggaagttatcctctgcctcccattctgaccacccttctcattccaacagtgagtctgtcagcgcaggtttagtttactcaatctccccttgcactaaagtatgtaaagtatgtaaacaggagacaggaaggtggtgcttacatccttaaaggcaccatctaatagcgggttactttcacatacagccctcccccagcagttgaatgacaacagaagcttcagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaaacaatcatttgctttttg

SEQ ID NO: 12 - protein, 777 aa, Homo sapiens
MDSKESLTPGREENPSSVLAQERGDVMDFYKTLRGGATVKVSASSPSLAVASQSDSKQRRLLVDFPKGSVSNAQQPDLSKAVSLSMGLYMGETETKVMGNDLGFPQQGQISLSSGETDLKLLEESIANLNRSTSVPENPKSSASTAVSAAPTEKEFPKTHSDVSSEQQHLKGQTGTNGGNVKLYTTDQSTFDILQDLEFSSGSPGKETNESPWRSDLLIDENCLLSPLAGEDDSFLLEGNSNEDCKPLILPDTKPKIKDNGDLVLSSPSNVTLPQVKTEKEDFIELCTPGVIKQEKLGTVYCQASFPGANIIGNKMSAISVHGVSTSGGQMYHYDMNTASLSQQQDQKPIFNVIPPIPVGSENWNRCQGSGDDNLTSLGTLNFPGRTVFSNGYSSPSMRPDVSSPPSSSSTATTGPPPKLCLVCSDEASGCHYGVLTCGSCKVFFKRAVEGQHNYLCAGRNDCIIDKIRRKNCPACRYRKCLQAGMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 13 - DNA, 768 bp, Homo sapiens
acaatctcacgagcgctcacaccttcccccgttatggtccttgaaaacattgaacctgaaattgtatatgcaggctatgacagctcaaaaccagatacagccgaaaatctgctctccacgctcaaccgcttagcaggcaaacagatgatccaagtcgtgaagtgggcaaaggtacttccaggatttaaaaacttgcctcttgaggaccaaattaccctaatccagtattcttggatgtgtctatcatcatttgccttgagctggagatcgtacaaacatacgaacagccaatttctctattttgcaccagacctagtctttaatgaagagaagatgcatcagtctgccatgtatgaactatgccaggggatgcaccaaatcagccttcagttcgttcgactgcagctcacctttgaagaatacaccatcatgaaagttttgctgctactaagcacaattccaaaggatggcctcaaaagccaggctgcatttgaagaaatgaggacaaattacatcaaagaactgaggaagatggtaactaagtgtcccaacaattctgggcagagctggcagaggttctaccaactgaccaagctgctggactccatgcatgacctggtgagcgacctgctggaattctgcttctacaccttccgagagtcccatgcgctgaaggtagagttccccgcaatgctggtggagatcatcagcgaccagctgcccaaggtggagtcggggaacgccaagccgctctacttccaccggaag

SEQ ID NO: 14 - protein, 256 aa, Homo sapiens
TISRALTPSPVMVLENIEPEIVYAGYDSSKPDTAENLLSTLNRLAGKQMIQVVKWAKVLPGFKNLPLEDQITLIQYSWMCLSSFALSWRSYKHTNSQFLYFAPDLVFNEEKMHQSAMYELCQGMHQISLQFVRLQLTFEEYTIMKVLLLLSTIPKDGLKSQAAFEEMRTNYIKELRKMVTKCPNNSGQSWQRFYQLTKLLDSMHDLVSDLLEFCFYTFRESHALKVEFPAMLVEIISDQLPKVESGNAKPLYFHRK

SEQ ID NO: 15 - DNA, 441 bp, Saccharomyces cerevisiae
atgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcg

SEQ ID NO: 16 - protein, 147 aa, Saccharomyces cerevisiae
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVS

SEQ ID NO: 17 - protein, 418 aa, Homo sapiens
  misc_feature (177)..(177): Xaa can be any naturally occurring amino acid
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSPEFGTVDDIPRAAGSTISRALTPSPVMVLXNIEPEIVYAGYDSSKPDTAENLLSTLNRLAGKQMIQVVKWAKVLPGFKNLPLEDQITLIQYSWMCLSSFALSWRSYKHTNSQFLYFAPDLVFNEEKMHQSAMYELCQGMHQISLQFVRLQLTFEEYTIMKVLLLLSTIPKDGLKSQAAFEEMRTNYIKELRKMVTKCPNNSGQSWQRFYQLTKLLDSMHDLVSDLLEFCFYTFRESHALKVEFPAMLVEIISDQLPKVESGNAKPLYFHRK

SEQ ID NO: 18 - DNA, 17 bp, Homo sapiens
cggagtactgtcctccg